SEQUENCE LISTING (142 sequences)

SEQ ID NO: 1 - protein, 10 aa, Homo sapiens
  Antigen
ASSLPTTMNY

SEQ ID NO: 2 - protein, 9 aa, Homo sapiens
  Antigen
HSEVGLPVY

SEQ ID NO: 3 - protein, 136 aa, Homo sapiens
  Alpha VJ region for Reference A
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASPVDRGSTLGRLYFGRGTQLTVW

SEQ ID NO: 4 - protein, 139 aa, Homo sapiens
  Alpha VJ region for Reference B
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEARQYSGAGSYQLTFGKGTKLSVI

SEQ ID NO: 5 - protein, 133 aa, Homo sapiens
  Beta VJ region for Reference A
MGCRLLCCAVLCLLGAVPMETGVTQTPRHLVMGMTNKKSLKCEQHLGHNAMYWYKQSAKKPLELMFVYNFKEQTENNSVPSRFSPECPNSSHLFLHLHTLQPEDSALYLCASSQVGTGSYEQYFGPGTRLTVT

SEQ ID NO: 6 - protein, 132 aa, Homo sapiens
  Beta VJ region for Reference B
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSLEWGPYEQYFGPGTRLTVT

SEQ ID NO: 7 - protein, 131 aa, Homo sapiens
  Alpha VJ region for Reference I
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENSGGYQKVTFGTGTKLQVI

SEQ ID NO: 8 - protein, 134 aa, Homo sapiens
  Alpha VJ region for Reference J
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREGYRDDKIIFGKGTRLHIL

SEQ ID NO: 9 - protein, 125 aa, Homo sapiens
  Alpha VJ region for Reference K
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAVNDYKLSFGAGTTVTVR

SEQ ID NO: 10 - protein, 125 aa, Homo sapiens
  Alpha VJ region for Reference L
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILPNAGNMLTFGGGTRLMVK

SEQ ID NO: 11 - protein, 128 aa, Homo sapiens
  Alpha VJ region for Reference M
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGPREYGNKLVFGAGTILRVK

SEQ ID NO: 12 - protein, 134 aa, Homo sapiens
  Alpha VJ region for Reference N
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDGKRVTGGGNKLTFGTGTQLKVE

SEQ ID NO: 13 - protein, 126 aa, Homo sapiens
  Alpha VJ region for Reference O
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCADAPGSSYKLIFGSGTRLLVR

SEQ ID NO: 14 - protein, 130 aa, Homo sapiens
  Beta VJ region for Reference I
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVGDHTIYFGEGSWLTVV

SEQ ID NO: 15 - protein, 133 aa, Homo sapiens
  Beta VJ region for Reference J
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSFSSGGAHEQFFGPGTRLTVL

SEQ ID NO: 16 - protein, 131 aa, Homo sapiens
  Beta VJ region for Reference K
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIGWNYEQYFGPGTRLTVT

SEQ ID NO: 17 - protein, 131 aa, Homo sapiens
  Beta VJ region for Reference L
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATRGTGTQPQHFGDGTRLSIL

SEQ ID NO: 18 - protein, 134 aa, Homo sapiens
  Beta VJ region for Reference M
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSVGGQGEVVQYFGPGTRLTVT

SEQ ID NO: 19 - protein, 132 aa, Homo sapiens
  Beta VJ region for Reference N
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLWRTGELFFGEGSRLTVL

SEQ ID NO: 20 - protein, 130 aa, Homo sapiens
  Beta VJ region for Reference O
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQVPHEQYFGPGTRLTVT

SEQ ID NO: 21 - protein, 16 aa, Homo sapiens
  CDR3 region of the alpha variable region for Reference A
CASPVDRGSTLGRLYF

SEQ ID NO: 22 - protein, 15 aa, Homo sapiens
  CDR3 region of the beta variable region for Reference A
CASSQVGTGSYEQYF

SEQ ID NO: 23 - protein, 19 aa, Homo sapiens
  CDR3 region of the alpha variable region for Reference B
CALSEARQYSGAGSYQLTF

SEQ ID NO: 24 - protein, 14 aa, Homo sapiens
  CDR3 region of the beta variable region for Reference B
CASSLEWGPYEQYF

SEQ ID NO: 25 - protein, 13 aa, Homo sapiens
  CDR3 region of the alpha variable region for Reference I
CAENSGGYQKVTF

SEQ ID NO: 26 - protein, 12 aa, Homo sapiens
  CDR3 region of the beta variable region for Reference I
CASSVGDHTIYF

SEQ ID NO: 27 - protein, 14 aa, Homo sapiens
  CDR3 region of the alpha variable region for Reference J
CAMREGYRDDKIIF

SEQ ID NO: 28 - protein, 15 aa, Homo sapiens
  CDR3 region of the beta variable region for Reference J
CASSFSSGGAHEQFF

SEQ ID NO: 29 - protein, 10 aa, Homo sapiens
  CDR3 region of the alpha variable region for Reference K
CAVNDYKLSF

SEQ ID NO: 30 - protein, 13 aa, Homo sapiens
  CDR3 region of the beta variable region for Reference K
CASSIGWNYEQYF

SEQ ID NO: 31 - protein, 12 aa, Homo sapiens
  CDR3 region of the alpha variable region for Reference L
CILPNAGNMLTF

SEQ ID NO: 32 - protein, 13 aa, Homo sapiens
  CDR3 region of the beta variable region for Reference L
CATRGTGTQPQHF

SEQ ID NO: 33 - protein, 13 aa, Homo sapiens
  CDR3 region of the alpha variable region for Reference M
CAGPREYGNKLVF

SEQ ID NO: 34 - protein, 15 aa, Homo sapiens
  CDR3 region of the beta variable region for Reference M
CASSVGGQGEVVQYF

SEQ ID NO: 35 - protein, 17 aa, Homo sapiens
  CDR3 region of the alpha variable region for Reference N
CATDGKRVTGGGNKLTF

SEQ ID NO: 36 - protein, 13 aa, Homo sapiens
  CDR3 region of the beta variable region for Reference N
CASSLWRTGELFF

SEQ ID NO: 37 - protein, 13 aa, Homo sapiens
  CDR3 region of the alpha variable region for Reference O
CADAPGSSYKLIF

SEQ ID NO: 38 - protein, 12 aa, Homo sapiens
  CDR3 region of the beta variable region for Reference O
CASSQVPHEQYF

SEQ ID NO: 39 - protein, 132 aa, Homo sapiens
  Alpha VJ region for Reference C
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVPRMDSSYKLIFGSGTRLLVRP

SEQ ID NO: 40 - protein, 140 aa, Homo sapiens
  Alpha VJ region for Reference D
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEARQYSGAGSYQLTFGKGTKLSVIP

SEQ ID NO: 41 - protein, 129 aa, Homo sapiens
  Alpha VJ region for Reference E
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGQGNRDDKIIFGKGTRLHILP

SEQ ID NO: 42 - protein, 131 aa, Homo sapiens
  Alpha VJ region for Reference F
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKDNNARLMFGDGTQLVVKP

SEQ ID NO: 43 - protein, 133 aa, Homo sapiens
  Alpha VJ region for Reference G
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATAVFNFGNEKLTFGTGTRLTIIP

SEQ ID NO: 44 - protein, 132 aa, Homo sapiens
  Alpha VJ region for Reference H
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENMGGAGKSTFGDGTTLTVKP

SEQ ID NO: 45 - protein, 134 aa, Homo sapiens
  Beta VJ region for Reference C
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSSTGARRSREQYFGPGTRLTVT

SEQ ID NO: 46 - protein, 132 aa, Homo sapiens; identical to SEQ ID NO: 6
  Beta VJ region for Reference D

SEQ ID NO: 47 - protein, 132 aa, Homo sapiens; identical to SEQ ID NO: 6
  Beta VJ region for Reference E

SEQ ID NO: 48 - protein, 132 aa, Homo sapiens
  Beta VJ region for Reference F
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLSSGLYEQYFGPGTRLTVT

SEQ ID NO: 49 - protein, 134 aa, Homo sapiens
  Beta VJ region for Reference G
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSSMTSGGPWEQYFGPGTRLTVT

SEQ ID NO: 50 - protein, 132 aa, Homo sapiens
  Beta VJ region for Reference H
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGGVAGVRQFFGPGTRLTVL

SEQ ID NO: 51 - protein, 14 aa, Homo sapiens
  CDR3 region of the alpha variable region for Reference C
CVVPRMDSSYKLIF

SEQ ID NO: 52 - protein, 16 aa, Homo sapiens
  CDR3 region of the beta variable region for Reference C
CASSSTGARRSREQYF

SEQ ID NO: 53 - protein, 19 aa, Homo sapiens
  CDR3 region of the alpha variable region for Reference D
CALSEARQYSGAGSYQLTF

SEQ ID NO: 54 - protein, 14 aa, Homo sapiens
  CDR3 region of the beta variable region for Reference D
CASSLEWGPYEQYF

SEQ ID NO: 55 - protein, 13 aa, Homo sapiens
  CDR3 region of the alpha variable region for Reference E
CAGQGNRDDKIIF

SEQ ID NO: 56 - protein, 14 aa, Homo sapiens
  CDR3 region of the beta variable region for Reference E
CASSLEWGPYEQYF

SEQ ID NO: 57 - protein, 12 aa, Homo sapiens
  CDR3 region of the alpha variable region for Reference F
CAVKDNNARLMF

SEQ ID NO: 58 - protein, 14 aa, Homo sapiens
  CDR3 region of the beta variable region for Reference F
CASSLSSGLYEQYF

SEQ ID NO: 59 - protein, 15 aa, Homo sapiens
  CDR3 region of the alpha variable region for Reference G
CATAVFNFGNEKLTF

SEQ ID NO: 60 - protein, 16 aa, Homo sapiens
  CDR3 region of the beta variable region for Reference G
CASSSMTSGGPWEQYF

SEQ ID NO: 61 - protein, 13 aa, Homo sapiens
  CDR3 region of the alpha variable region for Reference H
CAENMGGAGKSTF

SEQ ID NO: 62 - protein, 14 aa, Homo sapiens
  CDR3 region of the beta variable region for Reference H
CATSGGVAGVRQFF

SEQ ID NO: 63 - protein, 135 aa, Homo sapiens
  Alpha VJ region for Reference P
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREGYRDDKIIFGKGTRLHILP

SEQ ID NO: 64 - protein, 132 aa, Homo sapiens
  Alpha VJ region for Reference Q
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNSGAGSYQLTFGKGTKLSVIP

SEQ ID NO: 65 - protein, 132 aa, Homo sapiens
  Alpha VJ region for Reference R
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENSGGYQKVTFGTGTKLQVIP

SEQ ID NO: 66 - protein, 129 aa, Homo sapiens
  Alpha VJ region for Reference S
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGPREYGNKLVFGAGTILRVKS

SEQ ID NO: 67 - protein, 127 aa, Homo sapiens
  Alpha VJ region for Reference T
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVRAQGNARLMFGDGTQLVVKP

SEQ ID NO: 68 - protein, 126 aa, Homo sapiens
  Alpha VJ region for Reference U
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILPNAGNMLTFGGGTRLMVKP

SEQ ID NO: 69 - protein, 136 aa, Homo sapiens
  Alpha VJ region for Reference V
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRPYQGGSEKLVFGKGTKLTVNP

SEQ ID NO: 70 - protein, 132 aa, Homo sapiens
  Alpha VJ region for Reference W
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGPRWLTGGGNKLTFGTGTQLKVEL

SEQ ID NO: 71 - protein, 130 aa, Homo sapiens
  Alpha VJ region for Reference X
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAAPPPGYKYIFGTGTRLKVLA

SEQ ID NO: 72 - protein, 132 aa, Homo sapiens
  Alpha VJ region for Reference Y
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTELENYGQNFVFGPGTRLSVLP

SEQ ID NO: 73 - protein, 133 aa, Homo sapiens
  Alpha VJ region for Reference Z
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESSRNSGYALNFGKGTSLLVTP

SEQ ID NO: 74 - protein, 133 aa, Homo sapiens
  Alpha VJ region for Reference AA
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYYVPFNKFYFGSGTKLNVKP

SEQ ID NO: 75 - protein, 129 aa, Homo sapiens
  Alpha VJ region for Reference AB
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVTSGRLMFGDGTQLVVKP

SEQ ID NO: 76 - protein, 131 aa, Homo sapiens
  Alpha VJ region for Reference AC
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNKRGSYIPTFGRGTSLIVHP

SEQ ID NO: 77 - protein, 128 aa, Homo sapiens
  Alpha VJ region for Reference AD
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRGMEYGNKLVFGAGTILRVKS

SEQ ID NO: 78 - protein, 136 aa, Homo sapiens
  Alpha VJ region for Reference AE
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSGSGGSNYKLTFGKGTLLTVNP

SEQ ID NO: 79 - protein, 126 aa, Homo sapiens
  Alpha VJ region for Reference AF
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVTGGYQKVTFGTGTKLQVIP

SEQ ID NO: 80 - protein, 136 aa, Homo sapiens
  Alpha VJ region for Reference AG
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASAGNDMRFGAGTRLTVKP

SEQ ID NO: 81 - protein, 128 aa, Homo sapiens
  Alpha VJ region for Reference AH
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVTYNDMRFGAGTRLTVKP

SEQ ID NO: 82 - protein, 134 aa, Homo sapiens
  Alpha VJ region for Reference AI
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALIPSNDYKLSFGAGTTVTVRA

SEQ ID NO: 83 - protein, 133 aa, Homo sapiens; identical to SEQ ID NO: 15
  Beta VJ region for Reference P

SEQ ID NO: 84 - protein, 133 aa, Homo sapiens
  Beta VJ region for Reference Q
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPLGTGDYEQYFGPGTRLTVT

SEQ ID NO: 85 - protein, 130 aa, Homo sapiens; identical to SEQ ID NO: 14
  Beta VJ region for Reference R

SEQ ID NO: 86 - protein, 133 aa, Homo sapiens
  Beta VJ region for Reference S
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSYGGGSLVEQYFGPGTRLTVT

SEQ ID NO: 87 - protein, 131 aa, Homo sapiens
  Beta VJ region for Reference T
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCANAWGRNEQFFGPGTRLTVL

SEQ ID NO: 88 - protein, 131 aa, Homo sapiens; identical to SEQ ID NO: 17
  Beta VJ region for Reference U

SEQ ID NO: 89 - protein, 133 aa, Homo sapiens
  Beta VJ region for Reference V
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQGILAAGELFFGEGSRLTVL

SEQ ID NO: 90 - protein, 134 aa, Homo sapiens; identical to SEQ ID NO: 18
  Beta VJ region for Reference W

SEQ ID NO: 91 - protein, 131 aa, Homo sapiens
  Beta VJ region for Reference X
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASGEGDAYTQYFGPGTRLTVL

SEQ ID NO: 92 - protein, 134 aa, Homo sapiens
  Beta VJ region for Reference Y
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLSGGSGNTIYFGEGSWLTVV

SEQ ID NO: 93 - protein, 130 aa, Homo sapiens
  Beta VJ region for Reference Z
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVEDVPGGWGYTFGSGTRLTVV

SEQ ID NO: 94 - protein, 135 aa, Homo sapiens
  Beta VJ region for Reference AA
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSTTSGGGQETQYFGPGTRLLVL

SEQ ID NO: 95 - protein, 131 aa, Homo sapiens
  Beta VJ region for Reference AB
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLAAGEQYFGPGTRLTVT

SEQ ID NO: 96 - protein, 130 aa, Homo sapiens
  Beta VJ region for Reference AC
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSALGEQYFGPGTRLTVT

SEQ ID NO: 97 - protein, 133 aa, Homo sapiens
  Beta VJ region for Reference AD
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLGPGGSEAFFGQGTRLTVV

SEQ ID NO: 98 - protein, 126 aa, Homo sapiens
  Beta VJ region for Reference AE
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARSYEQYFGPGTRLTVT

SEQ ID NO: 99 - protein, 129 aa, Homo sapiens
  Beta VJ region for Reference AF
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVHRGVNTEAFFGQGTRLTVV

SEQ ID NO: 100 - protein, 131 aa, Homo sapiens
  Beta VJ region for Reference AG
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLGGYEQYFGPGTRLTVT

SEQ ID NO: 101 - protein, 131 aa, Homo sapiens
  Beta VJ region for Reference AH
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLLSGSGYTFGSGTRLTVV

SEQ ID NO: 102 - protein, 134 aa, Homo sapiens
  Beta VJ region for Reference AI
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYSMGEWSYEQYFGPGTRLTVT

SEQ ID NO: 103 - protein, 14 aa, Homo sapiens
  CDR3 region of the alpha variable region for Reference P
CAMREGYRDDKIIF

SEQ ID NO: 104 - protein, 15 aa, Homo sapiens
  CDR3 region of the beta variable region for Reference P
CASSFSSGGAHEQFF

SEQ ID NO: 105 - protein, 14 aa, Homo sapiens
  CDR3 region of the alpha variable region for Reference Q
CVVNSGAGSYQLTF

SEQ ID NO: 106 - protein, 15 aa, Homo sapiens
  CDR3 region of the beta variable region for Reference Q
CASSPLGTGDYEQYF

SEQ ID NO: 107 - protein, 13 aa, Homo sapiens
  CDR3 region of the alpha variable region for Reference R
CAENSGGYQKVTF

SEQ ID NO: 108 - protein, 12 aa, Homo sapiens
  CDR3 region of the beta variable region for Reference R
CASSVGDHTIYF

SEQ ID NO: 109 - protein, 13 aa, Homo sapiens
  CDR3 region of the alpha variable region for Reference S
CAGPREYGNKLVF

SEQ ID NO: 110 - protein, 15 aa, Homo sapiens
  CDR3 region of the beta variable region for Reference S
CASSYGGGSLVEQYF

SEQ ID NO: 111 - protein, 13 aa, Homo sapiens
  CDR3 region of the alpha variable region for Reference T
CAVRAQGNARLMF

SEQ ID NO: 112 - protein, 12 aa, Homo sapiens
  CDR3 region of the beta variable region for Reference T
CANAWGRNEQFF

SEQ ID NO: 113 - protein, 12 aa, Homo sapiens
  CDR3 region of the alpha variable region for Reference U
CILPNAGNMLTF

SEQ ID NO: 114 - protein, 13 aa, Homo sapiens
  CDR3 region of the beta variable region for Reference U
CATRGTGTQPQHF

SEQ ID NO: 115 - protein, 15 aa, Homo sapiens
  CDR3 region of the alpha variable region for Reference V
CAYRPYQGGSEKLVF

SEQ ID NO: 116 - protein, 15 aa, Homo sapiens
  CDR3 region of the beta variable region for Reference V
CASSQGILAAGELFF

SEQ ID NO: 117 - protein, 16 aa, Homo sapiens
  CDR3 region of the alpha variable region for Reference W
CAGPRWLTGGGNKLTF

SEQ ID NO: 118 - protein, 15 aa, Homo sapiens
  CDR3 region of the beta variable region for Reference W
CASSVGGQGEVVQYF

SEQ ID NO: 119 - protein, 12 aa, Homo sapiens
  CDR3 region of the alpha variable region for Reference X
CAAPPPGYKYIF

SEQ ID NO: 120 - protein, 13 aa, Homo sapiens
  CDR3 region of the beta variable region for Reference X
CASGEGDAYTQYF

SEQ ID NO: 121 - protein, 14 aa, Homo sapiens
  CDR3 region of the alpha variable region for Reference Y
CGTELENYGQNFVF

SEQ ID NO: 122 - protein, 15 aa, Homo sapiens
  CDR3 region of the beta variable region for Reference Y
CASSLSGGSGNTIYF

SEQ ID NO: 123 - protein, 14 aa, Homo sapiens
  CDR3 region of the alpha variable region for Reference Z
CAESSRNSGYALNF

SEQ ID NO: 124 - protein, 14 aa, Homo sapiens
  CDR3 region of the beta variable region for Reference Z
CSVEDVPGGWGYTF

SEQ ID NO: 125 - protein, 12 aa, Homo sapiens
  CDR3 region of the alpha variable region for Reference AA
CAYYVPFNKFYF

SEQ ID NO: 126 - protein, 16 aa, Homo sapiens
  CDR3 region of the beta variable region for Reference AA
CASSTTSGGGQETQYF

SEQ ID NO: 127 - protein, 10 aa, Homo sapiens
  CDR3 region of the alpha variable region for Reference AB
CAVTSGRLMF

SEQ ID NO: 128 - protein, 12 aa, Homo sapiens
  CDR3 region of the beta variable region for Reference AB
CASSLAAGEQYF

SEQ ID NO: 129 - protein, 13 aa, Homo sapiens
  CDR3 region of the alpha variable region for Reference AC
CVVNKRGSYIPTF

SEQ ID NO: 130 - protein, 11 aa, Homo sapiens
  CDR3 region of the beta variable region for Reference AC
CASSALGEQYF

SEQ ID NO: 131 - protein, 14 aa, Homo sapiens
  CDR3 region of the alpha variable region for Reference AD
CIVRGMEYGNKLVF

SEQ ID NO: 132 - protein, 14 aa, Homo sapiens
  CDR3 region of the beta variable region for Reference AD
CASSLGPGGSEAFF

SEQ ID NO: 133 - protein, 15 aa, Homo sapiens
  CDR3 region of the alpha variable region for Reference AE
CALSGSGGSNYKLTF

SEQ ID NO: 134 - protein, 10 aa, Homo sapiens
  CDR3 region of the beta variable region for Reference AE
CSARSYEQYF

SEQ ID NO: 135 - protein, 12 aa, Homo sapiens
  CDR3 region of the alpha variable region for Reference AF
CAVTGGYQKVTF

SEQ ID NO: 136 - protein, 13 aa, Homo sapiens
  CDR3 region of the beta variable region for Reference AF
CSVHRGVNTEAFF

SEQ ID NO: 137 - protein, 11 aa, Homo sapiens
  CDR3 region of the alpha variable region for Reference AG
CAASAGNDMRF

SEQ ID NO: 138 - protein, 12 aa, Homo sapiens
  CDR3 region of the beta variable region for Reference AG
CASSLGGYEQYF

SEQ ID NO: 139 - protein, 10 aa, Homo sapiens
  CDR3 region of the alpha variable region for Reference AH
CVVTYNDMRF

SEQ ID NO: 140 - protein, 13 aa, Homo sapiens
  CDR3 region of the beta variable region for Reference AH
CASSLLSGSGYTF

SEQ ID NO: 141 - protein, 13 aa, Homo sapiens
  CDR3 region of the alpha variable region for Reference AI
CALIPSNDYKLSF

SEQ ID NO: 142 - protein, 16 aa, Homo sapiens
  CDR3 region of the beta variable region for Reference AI
CASSYSMGEWSYEQYF

(19) United States
(12) Patent Application Publication
(10) Pub. No.: US 2022/0155321 A1
(43) Pub. Date: May 19, 2022

(54) SELECTION OF T CELL RECEPTORS

(71) Applicant: Gritstone bio, Inc, Emeryville, CA (US)

(72) Inventors: Matthew Joseph Davis, Emeryville, CA (US); Joshua Michael Francis, Emeryville, CA (US); Abubakar Jalloh, Emeryville, CA (US); Karin Jooss, Emeryville, CA (US); Christine Denise Palmer, Emeryville, CA (US); Mojca Skoberne, Emeryville, CA (US)

(73) Assignee: Gritstone bio, Inc, Emeryville, CA (US)

(21) Appl. No.: 17/433,817

(22) PCT Filed: Feb. 28, 2020
(86) PCT No.: PCT/US2020/020296

§ 371 Date: Aug. 25, 2021

Related U.S. Application Data
(60) Provisional application No. 62/812,572, filed on Mar. 1, 2019.

(51) Int. Cl. G01N 33/68; C12N 5/00; G01N 33/50; C12N 5/0783; G16B 40/20; G16B 15/30; C07K 14/725; G01N 15/14

(52) U.S. Cl. CPC G01N 33/6878; G01N 2015/1006; G01N 33/505; C12N 5/0636; G01N 33/5038; G16B 40/20; G16B 15/30; C07K 14/7051; G01N 15/1434; G01N 2333/70539; G01N 2333/70596; G01N 2333/57; G01N 2333/525; G01N 2333/55; G01N 2333/96436; G01N 2570/00; G01N 2333/005; C12N 5/0087

(57) ABSTRACT

Methods are provided to separately isolate antigen-binding T cells and antigen-activated T cells derived from a starting population of peripheral blood mononuclear cells, and to identify overlapping T cell receptor clonotypes. Antigens include personal and shared neoantigens as well as cancer-testis antigens. The T cell receptor clonotypes can be further used to develop cancer treatment therapies.

### RELATED APPLICATION

This application claims the benefit of U.S. Provisional Application No. 62/812,572, filed Mar. 1, 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 26, 2020, is named 14560-001-228_SEQ_LISTING.txt and is 106,016 bytes in size.

### FIELD OF THE INVENTION

The present disclosure relates to identification of antigen-specific T cell receptors.

### BACKGROUND OF THE INVENTION

Cancer involves a failure of immune surveillance to provide T cells capable of detecting and destroying clones of transformed cells which can grow into tumors. Research has focused on developing ways to engineer supplemental T cells that can recognize cancer-specific antigens and provide effective functional responses. Current approaches include efforts to cultivate and isolate antigen-specific, functionally responsive T cells from which T cell receptor sequences can be identified and used to engineer therapeutically effective T cell lines.

Such T cells are usually rare in starting peripheral blood mononuclear cell (PBMC) samples—in some cases fewer than 1 part in 10,000,000. As a result, current approaches often employ intensive stimulation (for example in vitro priming), expansion, and enrichment steps that are time consuming, expensive, and even then can yield low success rates. Contributing to the low success rates, T cell stimulation is known to downregulate expression of T cell receptors making detection more difficult. In addition, T cells are stimulated with target antigen at concentrations that can be much higher than concentrations expressed by cancer cells, resulting in selection of T cell receptors that fail to function at physiologically relevant concentrations of antigen.

The present disclosure overcomes these shortcomings by providing, inter alia, methods of identifying rare antigen-specific and functional T cells that avoid one or more limitations of in vitro priming and enable prequalification of T cell receptor candidates for development of T cell lines that are therapeutically effective at physiologically relevant concentrations of antigen.

### BRIEF SUMMARY OF THE INVENTION

Certain embodiments can provide, for example, a method for selection of T cell receptor clonotypes (for example rare T cell receptor clonotypes such as T cell receptor clonotypes with a frequency of less than 1 per 10,000,000 T cells in a sample of PBMCs). In certain embodiments, for example, the method can comprise: analyzing a mixture of T cells to identify antigen-binding T cells and antigen-activated T cells for a predetermined type of antigen (for example a neoantigen selected from a library of shared tumor neoantigens or a personalized neoantigen selected from an individual tumor cell). In certain embodiments, for example, the method can comprise: identifying at least a portion of at least one T cell receptor sequence shared by at least one of the antigen-binding T cells and at least one of the antigen-activated T cells.

A. In certain embodiments, for example, the analyzing can comprise analyzing a first portion of the mixture to identify the antigen-binding T cells and separately analyzing a second portion of the mixture to identify the antigen-activated T cells. In certain embodiments, for example, the analyzing the first portion of the mixture can comprise detecting one or more T cells bound to a P-loaded major histocompatibility complex (MHC) protein, wherein P is the predetermined type of antigen. In certain embodiments, for example, the P-loaded MHC can be coupled to a magnetic bead. In certain embodiments, for example, the detecting the one or more T cells bound to a P-loaded MHC protein can comprise isolating the one or more T cells bound to the P-loaded MHC protein via magnetic separation. In certain embodiments, for example, the P-loaded MHC protein can be coupled to a fluorophore. In certain embodiments, for example, the one or more T cells bound to the P-loaded MHC protein can be detected and isolated via fluorescence flow cytometry. In certain embodiments, for example, the detecting the one or more T cells bound to a P-loaded MHC protein can comprise passing the one or more T cells bound to the P-loaded MHC protein through a fluorescence flow cytometry device. In certain embodiments, for example, the MHC protein can be an MHC Class I protein. In certain embodiments, for example, the P-loaded MHC protein can be present in a P-loaded MHC protein multimer. In certain embodiments, for example, the separately analyzing a second portion of the mixture to identify the antigen-activated T cells can comprise detecting one or more T cells expressing one or more activation markers. In certain embodiments, for example, the detecting the one or more T cells expressing one or more activation markers can comprise isolating the one or more T cells expressing the one or more activation markers via magnetic separation. In certain embodiments, for example, the detecting the one or more T cells expressing one or more activation markers can comprise passing the one or more T cells expressing the one or more activation markers through a fluorescence flow cytometry device. In certain embodiments, for example, the method can be exclusive of in vitro priming.

B. In certain embodiments, for example, the predetermined type of antigen can be a peptide. In certain embodiments, for example, the peptide can consist of 8-15 (for example 8-12) amino acids. In certain embodiments, for example, the peptide can consist of 12-40 amino acids. In certain embodiments, for example, the predetermined type of antigen can be derived from a tumor (for example a solid tumor). In certain embodiments, for example, the predetermined type of antigen can be presented on a tumor. In certain embodiments, for example, the predetermined type of antigen can be a personalized antigen. In certain embodiments, for example, the predetermined type of antigen can be a shared tumor antigen (for example a shared tumor neoantigen) that has been observed in tumors across multiple subjects. In certain embodiments, for example, the shared tumor antigen can be a cancer/testis antigen. In certain embodiments, for example, the shared tumor antigen can be a cancer/testis-like antigen. In certain embodiments, for example, the shared tumor antigen can be a tumor associated peptide antigen. In certain embodiments, for example, the predetermined type of antigen can be characteristic of a particular type of tumor. In certain embodiments, for example, the predetermined type of antigen can be a tumor associated peptide antigen. In certain embodiments, for example, the predetermined type of antigen can be a viral antigen (for example an oncogenic viral protein such as HPV E6 and HPV E7). In certain embodiments, for example, the predetermined type of antigen can be a neoantigen. In certain embodiments, for example, the neoantigen can be a peptide. In certain embodiments, for example, the peptide can consist of 8-15 amino acids. In certain embodiments, for example, the peptide can consist of 12-40 amino acids. In certain embodiments, for example, the neoantigen can be derived from a tumor. In certain embodiments, for example, the tumor can be a solid tumor. In certain embodiments, for example, the neoantigen can be presented on a tumor. In certain embodiments, for example, the neoantigen can be a personalized neoantigen. In certain embodiments, for example, the neoantigen can be a shared tumor neoantigen. In certain embodiments, for example, the shared tumor neoantigen can be a tumor associated peptide neoantigen. In certain embodiments, for example, the neoantigen can be characteristic of a particular type of tumor. In certain embodiments, for example, the neoantigen can be a tumor associated peptide neoantigen. In certain embodiments, for example, the neoantigen can be selected from one or more neoantigens identified by a model. In certain embodiments, for example, the one or more neoantigens can be personalized neoantigens. In certain embodiments, for example, the one or more neoantigens can be present in a list of shared neoantigens. In certain embodiments, for example, the neoantigen can be selected from one or more neoantigens identified by an artificial intelligence model. In certain embodiments, for example, the model can be calibrated using machine learning. In certain embodiments, for example, the artificial intelligence model can comprise a neural network. In certain embodiments, for example, the neoantigen can be selected from a set of presentation likelihoods. In certain embodiments, for example, the neoantigen can be determined using one or more of the machine learning methods, software, and/or systems disclosed in the INCORPORATED REFERENCES.

C. In certain embodiments, for example, the at least a portion of at least one T cell receptor sequence can comprise at least one T cell receptor clonotype. In certain embodiments, for example, the at least a portion of at least one T cell receptor sequence can comprise at least one T cell receptor alpha chain, at least one T cell receptor beta chain, or at least one pair of T cell receptor alpha and beta chains. In certain embodiments, for example, the identifying can comprise: sequencing the at least one binding T cell at a single cell level. In certain embodiments, for example, the identifying can comprise: sequencing the at least one functional T cell at a single cell level. In certain embodiments, for example, the at least a portion of at least one T cell receptor sequence can comprise at least one CDR3 sequence.

D. In certain embodiments, for example, the at least one of the antigen-binding T cells and the at least one of the antigen-activated T cells can together be less than 1000 T cells (for example less than 100, less than 10, less than 5, less than 3, or 2) per 1,000,000 T cells present in the mixture of T cells.

E. In certain embodiments, for example, the method can further comprise: preparing the mixture of T cells, comprising: i) isolating, from a population of PBMCs, at least one T cell that binds to the predetermined type of antigen; and ii) expanding the isolated at least one T cell. In certain embodiments, for example, at least two T cells can bind to the predetermined type of antigen (i.e., the at least one T cell can be at least two T cells), wherein the expanding can comprise polyclonally expanding the at least two T cells. In certain embodiments, for example, the at least one of the antigen-binding T cells and the at least one of the antigen-activated T cells can together be less than 1000 T cells (for example less than 100, less than 10, less than 5, less than 3, or 2) per 10,000,000 T cells present in the population of PBMCs. In certain embodiments, for example, the mixture of T cells can be a product of in vitro priming.

Certain embodiments can provide, for example, a method for selection of shared receptor sequences in lymphocytes. In certain embodiments, for example, the method can comprise: analyzing a mixture of lymphocytes to identify stimulated lymphocytes and costimulated lymphocytes for a predetermined type of antigen. In certain embodiments, for example, the method can comprise: identifying at least a portion of at least one receptor sequence shared by at least one of the stimulated lymphocytes and at least one of the costimulated lymphocytes.

A. In certain embodiments, for example, the mixture of stimulated lymphocytes and costimulated lymphocytes can be T cells. In certain embodiments, for example, the mixture of stimulated lymphocytes and costimulated lymphocytes can be B cells. In certain embodiments, for example, the mixture of stimulated lymphocytes and costimulated lymphocytes can be natural killer cells.

B. In certain embodiments, for example, the analyzing can comprise analyzing a first portion of the mixture to identify the stimulated lymphocytes and separately analyzing a second portion of the mixture to identify the costimulated lymphocytes. In certain embodiments, for example, the analyzing the first portion of the mixture can comprise detecting one or more stimulated lymphocytes bound to a protein, wherein the protein comprises (for example can be integral to or complexed with) the predetermined type of antigen. In certain embodiments, for example, the protein can be coupled to a magnetic bead. In certain embodiments, for example, the detecting the one or more stimulated lymphocytes bound to the protein can comprise isolating the one or more stimulated lymphocytes bound to the protein via magnetic separation. In certain embodiments, for example, the protein can be coupled to a fluorophore. In certain embodiments, for example, the one or more stimulated lymphocytes bound to the protein can be detected and isolated via fluorescence flow cytometry. In certain embodiments, for example, the detecting the one or more stimulated lymphocytes bound to the protein can comprise passing the one or more stimulated lymphocytes bound to the protein through a fluorescence flow cytometry device. In certain embodiments, for example, the separately analyzing a second portion of the mixture to identify the costimulated lymphocytes can comprise detecting one or more stimulated lymphocytes expressing one or more markers. In certain embodiments, for example, the detecting the one or more stimulated lymphocytes expressing one or more markers can comprise isolating the one or more stimulated lymphocytes expressing the one or more markers via magnetic separation. In certain embodiments, for example, the detecting the one or more stimulated lymphocytes expressing one or more markers can comprise passing the one or more stimulated lymphocytes expressing the one or more markers through a fluorescence flow cytometry device. In certain embodiments, for example, the method can be exclusive of priming (for example in vitro priming) with professional antigen presenting cells.

C. In certain embodiments, for example, the predetermined type of antigen can be a peptide. In certain embodiments, for example, the peptide can consist of 8-15 (for example 8-12) amino acids. In certain embodiments, for example, the peptide can consist of 12-40 amino acids. In certain embodiments, for example, the predetermined type of antigen can be derived from a tumor (for example a solid tumor). In certain embodiments, for example, the predetermined type of antigen can be presented on a tumor. In certain embodiments, for example, the predetermined type of antigen can be a personalized antigen. In certain embodiments, for example, the predetermined type of antigen can be a shared tumor antigen (for example a shared tumor neoantigen). In certain embodiments, for example, the shared tumor antigen can be a cancer/testis antigen. In certain embodiments, for example, the shared tumor antigen can be a cancer/testis-like antigen. In certain embodiments, for example, the shared tumor antigen can be a tumor associated peptide antigen. In certain embodiments, for example, the predetermined type of antigen can be characteristic of a particular type of tumor. In certain embodiments, for example, the predetermined type of antigen can be a tumor associated peptide antigen. In certain embodiments, for example, the predetermined type of antigen can be a viral antigen (for example an oncogenic viral protein such as HPV E6 and HPV E7). In certain embodiments, for example, the predetermined type of antigen can be a neoantigen. In certain embodiments, for example, the neoantigen can be a peptide. In certain embodiments, for example, the peptide can consist of 8-15 amino acids. In certain embodiments, for example, the peptide can consist of 12-40 amino acids. In certain embodiments, for example, the neoantigen can be derived from a tumor. In certain embodiments, for example, the tumor can be a solid tumor. In certain embodiments, for example, the neoantigen can be presented on a tumor. In certain embodiments, for example, the neoantigen can be a personalized neoantigen. In certain embodiments, for example, the neoantigen can be a shared tumor neoantigen. In certain embodiments, for example, the shared tumor neoantigen can be a tumor associated peptide neoantigen. In certain embodiments, for example, the neoantigen can be characteristic of a particular type of tumor. In certain embodiments, for example, the neoantigen can be a tumor associated peptide neoantigen. In certain embodiments, for example, the neoantigen can be selected from one or more neoantigens identified by a model. In certain embodiments, for example, the one or more neoantigens can be personalized neoantigens. In certain embodiments, for example, the one or more neoantigens can be present in a list of shared neoantigens. In certain embodiments, for example, the neoantigen can be selected from one or more neoantigens identified by an artificial intelligence model. In certain embodiments, for example, the model can be calibrated using machine learning. In certain embodiments, for example, the artificial intelligence model can comprise a neural network. In certain embodiments, for example, the neoantigen can be selected from a set of presentation likelihoods. In certain embodiments, for example, the neoantigen can be determined using one or more of the machine learning methods, software, and/or systems disclosed in the INCORPORATED REFERENCES.

D. In certain embodiments, for example, the at least a portion of at least one receptor sequence can comprise at least one receptor clonotype. In certain embodiments, for example, the at least a portion of at least one receptor sequence can comprise at least one receptor alpha chain, at least one receptor beta chain, or at least one pair of receptor alpha and beta chains. In certain embodiments, for example, the identifying can comprise: sequencing the at least one of the stimulated lymphocytes at a single cell level. In certain embodiments, for example, the identifying can comprise: sequencing the at least one of the costimulated lymphocytes at a single cell level. In certain embodiments, for example, the at least a portion of at least one receptor sequence can comprise at least one antigen recognition sequence.

E. In certain embodiments, for example, the at least one of the stimulated lymphocytes and the at least one of the costimulated lymphocytes can together be less than 1000 T cells (for example less than 100, less than 10, less than 5, less than 3, or 2) per 1,000,000 T cells present in the mixture of lymphocytes.

F. In certain embodiments, for example, the method can further comprise: preparing the mixture of lymphocytes, comprising: i) isolating, from a population of PBMCs, at least one lymphocyte that binds to the predetermined type of antigen; and ii) expanding the isolated at least one lymphocyte. In certain embodiments, for example, the at least two lymphocytes can bind to the predetermined type of antigen (i.e., the at least one lymphocyte can be at least two lymphocytes), wherein the expanding can comprise polyclonally expanding the at least two lymphocytes. In certain embodiments, for example, the at least one of the stimulated lymphocytes and the at least one of the costimulated lymphocytes can together be less than 1000 T cells (for example less than 100, less than 10, less than 5, less than 3, or 2) per 10,000,000 lymphocytes present in the population of PBMCs. In certain embodiments, for example, the mixture of lymphocytes can be a product of priming (for example in vitro priming) with professional antigen presenting cells.

Certain embodiments can provide, for example, a method for selection of T cell receptor clonotypes. In certain embodiments, for example, the method can comprise: analyzing a mixture of naïve T cells to identify antigen-binding T cells and functional T cells for a predetermined type of antigen. In certain embodiments, for example, the method can comprise: identifying at least a portion of at least one T cell receptor sequence shared by at least one of the antigen-binding T cells and at least one of the functional T cells.

Certain embodiments can provide, for example, a method for selection of T cell receptors. In certain embodiments, for example, the method can comprise: binding at least a first antigen-binding T cell to at least a first one of a predetermined type of antigen, comprising: contacting a first plurality of T cells (for example a first plurality of T cells containing the at least a first antigen-binding T cell) with the first one of the predetermined type of antigen. In certain embodiments, for example, the method can comprise: activating at least a first functional T cell, comprising: contacting a second plurality of T cells (for example a second plurality of T cells containing the at least a first functional T cell) with a plurality of cells that present at least a second one of the predetermined type of antigen (for example a plurality of cells that present a physiologically relevant concentration of the predetermined type of antigen). In certain embodiments, for example, the method can comprise: identifying at least a portion of at least one T cell receptor sequence that is common to the at least one antigen-binding T cell and the at least one functional T cell.

A. In certain embodiments, for example, the plurality of cells that present at least the second one of the predetermined type of antigen can present a plurality of the predetermined type of antigen within a predetermined concentration range (or a single predetermined concentration value). In certain embodiments, for example, the plurality of cells that present at least the second one of the predetermined type of antigen can be prepared by pulsing the plurality of cells with a quantity of the predetermined type of antigen (for example to form a P-loaded plurality of cells, where P is the predetermined type of antigen). In certain embodiments, for example, the plurality of cells that present at least the second one of the predetermined type of antigen can be prepared by pulsing the plurality of cells with a solution containing the predetermined type of antigen for a predetermined period of time, the solution containing the predetermined type of antigen at a concentration of between 0.000001 μM and 100 μM, for example a concentration of between 0.000001 μM and 0.00001 μM, for example a concentration of between 0.00001 μM and 0.0001 μM, between 0.0001 μM and 0.001 μM, between 0.001 and 0.01 μM, between 0.01 and 0.1 μM, between 0.0001 μM and 100 μM, between 0.001 μM and 100 μM, between 0.01 μM and 10 μM, between 0.1 μM and 10 μM, between 1 μM and 100 μM, between 1 μM and 50 μM, between 1 μM and 25 μM, between 5 μM and 25 μM, between 10 μM and 100 μM, or between 10 μM and 30 μM. In certain embodiments, for example, the solution can contain the predetermined type of antigen at a concentration of less than 100 μM, for example a concentration of less than 75 μM, less than 50 μM, less than 25 μM, less than 10 μM, or less than 1 μM. In any of the foregoing embodiments, for example, the predetermined period of time can be between 1 hour and 36 hours, for example between 6 hours and 24 hours, between 6 hours and 12 hours, between 12 hours and 24 hours, or the predetermined period of time can be between 9 hours and 18 hours. In any of the foregoing embodiments, for example, the predetermined period of time can be at least 1 hour, at least 4 hours, at least 8 hours, at least 12 hours, at least 18 hours, or the predetermined period of time can be at least 24 hours. In any of the foregoing embodiments, for example, the predetermined period of time can be less than 168 hours, less than 72 hours, less than 36 hours, less than 24 hours, or the predetermined period of time can be less than 12 hours. In certain embodiments, for example, the predetermined concentration range (or predetermined concentration value) can be based on an expected concentration of the predetermined type of antigen in a tumor (for example an expected concentration of the predetermined type of antigen expressed on the surface of a tumor).

B. In certain embodiments, for example, the binding can comprise binding the at least a first binding T cell to a P-loaded MHC protein, wherein P is the predetermined type of antigen. In certain embodiments, for example, the MHC protein can be an MHC Class I protein. In certain embodiments, for example, the P-loaded MHC protein can be present in a P-loaded MHC protein multimer.

C. In certain embodiments, for example, the first plurality of T cells and the second plurality of T cells can be derived from a common population of PBMCs. In certain embodiments, for example, the first plurality of T cells and the second plurality of T cells can be derived from one or more healthy donors. In certain embodiments, for example, the one or more healthy donors can be at least partially human leukocyte antigen (HLA)-matched to a subject. In certain embodiments, for example, the one or more healthy donors can be at least partially HLA-matched to a subject for presenting the predetermined type of antigen. In certain embodiments, for example, the one or more healthy donors can be matched to a subject for HLA-A. In certain embodiments, for example, the one or more healthy donors can be matched to a subject for HLA-B. In certain embodiments, for example, the one or more healthy donors can be matched to a subject for HLA-C. In certain embodiments, for example, the one or more healthy donors can be matched to a subject for HLA-DP. In certain embodiments, for example, the one or more healthy donors can be matched to a subject for HLA-DQ. In certain embodiments, for example, the one or more healthy donors can be matched to a subject for HLA-DR. In certain embodiments, for example, the one or more healthy donors can be matched to a subject for HLA-A, HLA-B, HLA-C, HLA-DP, HLA-DQ, HLA-DR, or a combination of two or more of the foregoing. In certain embodiments, for example, the one or more healthy donors can be at least partially HLA-mismatched to a subject. In certain embodiments, for example, the one or more healthy donors can be completely HLA-mismatched to a subject. In certain embodiments, for example, the one or more healthy donors can be selectively HLA-mismatched to a subject. In certain embodiments, for example, the one or more healthy donors can be mismatched to a subject for HLA-B. In certain embodiments, for example, the one or more healthy donors can be mismatched to a subject for HLA-C. In certain embodiments, for example, the one or more healthy donors can be mismatched to a subject for HLA-DP. In certain embodiments, for example, the one or more healthy donors can be mismatched to a subject for HLA-DQ. In certain embodiments, for example, the one or more healthy donors can be mismatched to a subject for HLA-DR. In certain embodiments, for example, the one or more healthy donors can be mismatched to a subject for HLA-A, HLA-B, HLA-C, HLA-DP, HLA-DQ, HLA-DR, or a combination of two or more of the foregoing.

In certain embodiments, for example, the one or more healthy donors can be at least partially HLA-matched to a predicted HLA for presenting the predetermined type of antigen (for example an HLA predicted in combination with the predetermined type of antigen by one of the machine learning models and/or methods disclosed herein or in one of the INCORPORATED REFERENCES to present the predetermined type of antigen, for example predicted for a predetermined type of cancer). In certain embodiments, for example, the predicted HLA can be selected from the group consisting of HLA-A, HLA-B, HLA-C, HLA-DP, HLA-DQ, HLA-DR, or a combination of two or more of the foregoing.

In certain embodiments, for example, the one or more healthy donors can be at least partially HLA-mismatched to a predicted HLA for presenting the predetermined type of antigen (for example an HLA predicted in combination with the predetermined type of antigen by one of the machine learning models and/or methods disclosed herein or in one of the INCORPORATED REFERENCES to present the predetermined type of antigen, for example predicted for a predetermined type of cancer). In certain embodiments, for example, the predicted HLA can be selected from the group consisting of HLA-A, HLA-B, HLA-C, HLA-DP, HLA-DQ, HLA-DR, or a combination of two or more of the foregoing.

D. In certain embodiments, for example, the first plurality of T cells and the second plurality of T cells can be (or can comprise or can be derived from) naïve CD8⁺ T cells. In certain embodiments, for example, the first plurality of T cells and the second plurality of T cells can be (or can comprise or can be derived from) naïve T cells. In certain embodiments, for example, the first plurality of T cells and the second plurality of T cells can be (or can comprise or can be derived from) memory T cells. In certain embodiments, for example, the first plurality of T cells and the second plurality of T cells can be (or can comprise or can be derived from) CD8⁺ T cells. In certain embodiments, for example, the first plurality of T cells and the second plurality of T cells can be (or can comprise or can be derived from) CD4⁺ T cells. In certain embodiments, for example, the first plurality of T cells and the second plurality of T cells can be (or can comprise or can be derived from) CD4⁺ CD8⁺ T cells. In certain embodiments, for example, the first plurality of T cells and the second plurality of T cells can be (or can comprise or can be derived from) CD4-CD8⁺ T cells. In certain embodiments, for example, the first plurality of T cells and the second plurality of T cells can be (or can comprise or can be derived from) CD4⁺ CD8⁻ T cells.

E. In certain embodiments, for example, the plurality of cells that present at least the second one of the predetermined type of antigen can comprise one or more tumor cells. In certain embodiments, for example, the plurality of cells that present at least the second one of the predetermined type of antigen can comprise one or more dendritic cells. In certain embodiments, for example, the plurality of cells that present at least the second one of the predetermined type of antigen can comprise one or more antigen presenting cells (for example one or more professional antigen presenting cells). In certain embodiments, for example, the plurality of cells that present at least the second one of the predetermined type of antigen can comprise one or more artificial antigen presenting cells. In certain embodiments, for example, the plurality of cells that present at least the second one of the predetermined type of antigen can comprise one or more macrophages. In certain embodiments, for example, the plurality of cells that present at least the second one of the predetermined type of antigen can comprise one or more monocytes. In certain embodiments, for example, the plurality of cells that present at least the second one of the predetermined type of antigen can comprise one or more B cells. In certain embodiments, for example, the plurality of cells that present at least the second one of the predetermined type of antigen can comprise one or more the plurality of cells that present at least the second one of the predetermined type of antigen express the predetermined type of antigen.

F. In certain embodiments, for example, the method can further comprise: detecting the binding via flow cytometry (for example fluorescence flow cytometry). In certain embodiments, for example, the first one of the predetermined type of antigen can be coupled to a magnetic bead, where the method can further comprise: detecting the at least a first antigen-binding T cell via magnetic separation. In certain embodiments, for example, the method can further comprise: detecting the activating via flow cytometry (for example fluorescence flow cytometry). In certain embodiments, for example, the method can further comprise: detecting the at least a first functional T cell via magnetic separation.

G. In certain embodiments, for example, the method can further comprise: detecting the activating, comprising: detecting one or more biomarkers. In certain embodiments, for example, the one or more biomarkers can comprise CD137. In certain embodiments, for example, the method can further comprise: detecting the activating, comprising: detecting presence of one or more molecules indicative of T cell activation. In certain embodiments, for example, the one or more molecules can comprise interferon gamma. In certain embodiments, for example, the method can further comprise: detecting the activating, comprising: detecting T cell proliferation. In certain embodiments, for example, the activating at least a first functional T cell can be a T cell present in the second plurality of T cells. In certain embodiments, for example, the activating at least a first functional T cell can be a T cell formed by proliferation of one of the T cells present in the second plurality of T cells.

H. In certain embodiments, for example, the predetermined type of antigen can be a peptide. In certain embodiments, for example, the peptide can consist of 8-15 (for example 8-12) amino acids. In certain embodiments, for example, the peptide can consist of 12-40 amino acids. In certain embodiments, for example, the predetermined type of antigen can be derived from a tumor (for example a solid tumor). In certain embodiments, for example, the predetermined type of antigen can be presented on a tumor. In certain embodiments, for example, the predetermined type of antigen can be a personalized antigen. In certain embodiments, for example, the predetermined type of antigen can be a shared tumor antigen (for example a shared tumor neoantigen). In certain embodiments, for example, the shared tumor antigen can be a cancer/testis antigen. In certain embodiments, for example, the shared tumor antigen can be a cancer/testis-like antigen. In certain embodiments, for example, the shared tumor antigen can be a tumor associated peptide antigen. In certain embodiments, for example, the predetermined type of antigen can be characteristic of a particular type of tumor. In certain embodiments, for example, the predetermined type of antigen can be a tumor associated peptide antigen. In certain embodiments, for example, the predetermined type of antigen can be a viral antigen (for example an oncogenic viral protein such as HPV E6 and HPV E7). In certain embodiments, for example, the predetermined type of antigen can be a neoantigen. In certain embodiments, for example, the neoantigen can be a peptide. In certain embodiments, for example, the peptide can consist of 8-15 amino acids. In certain embodiments, for example, the peptide can consist of 12-40 amino acids. In certain embodiments, for example, the neoantigen can be derived from a tumor. In certain embodiments, for example, the tumor can be a solid tumor. In certain embodiments, for example, the neoantigen can be presented on a tumor. In certain embodiments, for example, the neoantigen can be a personalized neoantigen. In certain embodiments, for example, the neoantigen can be a shared tumor neoantigen. In certain embodiments, for example, the shared tumor neoantigen can be a tumor associated peptide neoantigen. In certain embodiments, for example, the neoantigen can be characteristic of a particular type of tumor. In certain embodiments, for example, the neoantigen can be a tumor associated peptide neoantigen. In certain embodiments, for example, the neoantigen can be selected from one or more neoantigens identified by a model. In certain embodiments, for example, the one or more neoantigens can be personalized neoantigens. In certain embodiments, for example, the one or more neoantigens can be present in a list of shared neoantigens. In certain embodiments, for example, the neoantigen can be selected from one or more neoantigens identified by an artificial intelligence model. In certain embodiments, for example, the model can be calibrated using machine learning. In certain embodiments, for example, the artificial intelligence model can comprise a neural network. In certain embodiments, for example, the neoantigen can be selected from a set of presentation likelihoods. In certain embodiments, for example, the neoantigen can be determined using one or more of the machine learning methods, software, and/or systems disclosed in the INCORPORATED REFERENCES.

Certain embodiments can provide, for example, a method for selection of T cell receptors. In certain embodiments, for example, the method can comprise: binding at least a first antigen-binding T cell present in a first plurality of T cells to at least a first one of a Class I P-MHC protein multimer, which P is a predetermined type of antigen, comprising: contacting the first plurality of T cells with the first one of the Class I P-MHC protein multimer. In certain embodiments, for example, the method can comprise: activating at least a first functional T cell present in a second plurality of T cells, comprising: contacting the second plurality of T cells with a plurality of cells that present at least a first one of a Class II P-MHC protein multimer. In certain embodiments, for example, the method can comprise: identifying at least a portion of at least one T cell receptor sequence that is common to the at least one antigen-binding T cell and the at least one functional T cell.

Certain embodiments can provide, for example, a method for selection of T cell receptors. In certain embodiments, for example, the method can comprise: binding at least a first antigen-binding T cell present in a first plurality of T cells to at least a first one of a Class I P-MHC protein multimer, which P is a predetermined type of antigen, comprising: contacting the first plurality of T cells with the first one of the Class I P-MHC protein multimer. In certain embodiments, for example, the method can comprise: activating at least a first functional T cell present in a second plurality of T cells, comprising: contacting the second plurality of T cells with a plurality of cells that present at least a first Class I P-MHC protein. In certain embodiments, for example, the method can comprise: identifying at least a portion of at least one T cell receptor sequence that is common to the at least one antigen-binding T cell and the at least one functional T cell.

Certain embodiments can provide, for example, a method for selection of T cell receptors (for example a method exclusive of any of the in vitro priming methods disclosed herein or in one of the INCORPORATED REFERENCES). In certain embodiments, for example, the method can comprise: isolating a first T cell from a plurality of T cells, the first T cell bound to a P-loaded MHC protein, which P is a predetermined type of antigen. In certain embodiments, for example, the method can comprise: further isolating a second T cell from the plurality of T cells, the second T cell expressing at least one biomarker indicative of activation by the predetermined type of antigen. In certain embodiments, for example, the method can comprise: matching at least a portion of a T cell receptor sequence of the first T cell with at least a portion of a T cell receptor sequence of the second T cell.

A. In certain embodiments, for example, the method can further comprise: deriving the plurality of T cells from at least two T cells that are separately bound to at least two P-loaded MHC proteins. In certain embodiments, for example, the deriving can comprise expanding the at least a first T cell and the at least a second T cell. In certain embodiments, for example, the expanding can comprise polyclonally expanding the at least a first T cell and the at least a second T cell. In certain embodiments, for example, the at least a first T cell and the at least a second T cell can be in a mixture during the expanding. In certain embodiments, for example, the at least a first T cell and the at least a second T cell can be separated from one another prior to the expanding.

B. In certain embodiments, for example, the predetermined type of antigen can be a peptide. In certain embodiments, for example, the peptide can consist of 8-15 (for example 8-12) amino acids. In certain embodiments, for example, the peptide can consist of 12-40 amino acids. In certain embodiments, for example, the predetermined type of antigen can be derived from a tumor (for example a solid tumor). In certain embodiments, for example, the predetermined type of antigen can be presented on a tumor. In certain embodiments, for example, the predetermined type of antigen can be a personalized antigen. In certain embodiments, for example, the predetermined type of antigen can be a shared tumor antigen (for example a shared tumor neoantigen). In certain embodiments, for example, the shared tumor antigen can be a cancer/testis antigen. In certain embodiments, for example, the shared tumor antigen can be a cancer/testis-like antigen. In certain embodiments, for example, the shared tumor antigen can be a tumor associated peptide antigen. In certain embodiments, for example, the predetermined type of antigen can be characteristic of a particular type of tumor. In certain embodiments, for example, the predetermined type of antigen can be a tumor associated peptide antigen. In certain embodiments, for example, the predetermined type of antigen can be a viral antigen (for example an oncogenic viral protein such as HPV E6 and HPV E7). In certain embodiments, for example, the predetermined type of antigen can be a neoantigen. In certain embodiments, for example, the neoantigen can be a peptide. In certain embodiments, for example, the peptide can consist of 8-15 amino acids. In certain embodiments, for example, the peptide can consist of 12-40 amino acids. In certain embodiments, for example, the neoantigen can be derived from a tumor. In certain embodiments, for example, the tumor can be a solid tumor. In certain embodiments, for example, the neoantigen can be presented on a tumor. In certain embodiments, for example, the neoantigen can be a personalized neoantigen. In certain embodiments, for example, the neoantigen can be a shared tumor neoantigen. In certain embodiments, for example, the shared tumor neoantigen can be a tumor associated peptide neoantigen. In certain embodiments, for example, the neoantigen can be characteristic of a particular type of tumor. In certain embodiments, for example, the neoantigen can be a tumor associated peptide neoantigen. In certain embodiments, for example, the neoantigen can be selected from one or more neoantigens identified by a model. In certain embodiments, for example, the one or more neoantigens can be personalized neoantigens. In certain embodiments, for example, the one or more neoantigens can be present in a list of shared neoantigens. In certain embodiments, for example, the neoantigen can be selected from one or more neoantigens identified by an artificial intelligence model. In certain embodiments, for example, the model can be calibrated using machine learning. In certain embodiments, for example, the artificial intelligence model can comprise a neural network. In certain embodiments, for example, the neoantigen can be selected from a set of presentation likelihoods. In certain embodiments, for example, the neoantigen can be determined using one or more of the machine learning methods, software, and/or systems disclosed in the INCORPORATED REFERENCES.

Certain embodiments can provide, for example, a method for detecting functional T cell receptor clonotypes. In certain embodiments, for example, the method can comprise: isolating, from a population of PBMCs, at least one T cell that binds to a predetermined type of antigen. In certain embodiments, for example, the method can comprise: forming a plurality of cognate T cells, comprising: expanding the isolated at least one T cell. In certain embodiments, for example, the method can comprise: activating at least a first functional T cell, comprising: contacting T cells derived from the plurality of cognate T cells with at least one of a plurality of activation agents that is immunogenic for the predetermined type of antigen. In certain embodiments, for example, the method can comprise: confirming that the at least a first functional T cell is configured to bind to a P-loaded MHC protein, which P is the predetermined type of antigen.

A. In certain embodiments, for example, the forming can comprise indirect T cell receptor cross-linking. In certain embodiments, for example, the forming can be limited to a single polyclonal expansion. In certain embodiments, for example, the forming can comprise multiple polyclonal expansions. In certain embodiments, for example, at least one of the multiple polyclonal expansions can be followed by isolating at least one further T cell that binds to the predetermined type of antigen.

B. In certain embodiments, for example, the at least a first functional T cell can have a dissociation constant with the P-loaded MHC protein of less than 50 μM. In certain embodiments, for example, the at least a first functional T cell can have a half-life with the P-loaded MHC protein of between 0.01 seconds and 100 seconds (for example between 2 seconds and 10 seconds). In certain embodiments, for example, the predetermined type of antigen can be a tumor associated peptide antigen, wherein the at least a first functional T cell has: i) a dissociation constant with the P-loaded MHC protein of less than 50 μM; and ii) a half-life with the P-loaded MHC protein of 0.01 seconds and 100 seconds (for example between 2 seconds and 10 seconds).

C. In certain embodiments, for example, the least one of the plurality of activation agents can be antigenic for the predetermined type of antigen. In certain embodiments, for example, the at least one T cell can have undergone negative selection.

D. In certain embodiments, for example, the predetermined type of antigen can be a peptide. In certain embodiments, for example, the peptide can consist of 8-15 (for example 8-12) amino acids. In certain embodiments, for example, the peptide can consist of 12-40 amino acids. In certain embodiments, for example, the predetermined type of antigen can be derived from a tumor (for example a solid tumor). In certain embodiments, for example, the predetermined type of antigen can be presented on a tumor. In certain embodiments, for example, the predetermined type of antigen can be a personalized antigen. In certain embodiments, for example, the predetermined type of antigen can be a shared tumor antigen (for example a shared tumor neoantigen). In certain embodiments, for example, the shared tumor antigen can be a cancer/testis antigen. In certain embodiments, for example, the shared tumor antigen can be a cancer/testis-like antigen. In certain embodiments, for example, the shared tumor antigen can be a tumor associated peptide antigen. In certain embodiments, for example, the predetermined type of antigen can be characteristic of a particular type of tumor. In certain embodiments, for example, the predetermined type of antigen can be a tumor associated peptide antigen. In certain embodiments, for example, the predetermined type of antigen can be a viral antigen (for example an oncogenic viral protein such as HPV E6 and HPV E7). In certain embodiments, for example, the predetermined type of antigen can be a neoantigen. In certain embodiments, for example, the neoantigen can be a peptide. In certain embodiments, for example, the peptide can consist of 8-15 amino acids. In certain embodiments, for example, the peptide can consist of 12-40 amino acids. In certain embodiments, for example, the neoantigen can be derived from a tumor. In certain embodiments, for example, the tumor can be a solid tumor. In certain embodiments, for example, the neoantigen can be presented on a tumor. In certain embodiments, for example, the neoantigen can be a personalized neoantigen. In certain embodiments, for example, the neoantigen can be a shared tumor neoantigen. In certain embodiments, for example, the shared tumor neoantigen can be a tumor associated peptide neoantigen. In certain embodiments, for example, the neoantigen can be characteristic of a particular type of tumor. In certain embodiments, for example, the neoantigen can be a tumor associated peptide neoantigen. In certain embodiments, for example, the neoantigen can be selected from one or more neoantigens identified by a model. In certain embodiments, for example, the one or more neoantigens can be personalized neoantigens. In certain embodiments, for example, the one or more neoantigens can be present in a list of shared neoantigens. In certain embodiments, for example, the neoantigen can be selected from one or more neoantigens identified by an artificial intelligence model. In certain embodiments, for example, the model can be calibrated using machine learning. In certain embodiments, for example, the artificial intelligence model can comprise a neural network. In certain embodiments, for example, the neoantigen can be selected from a set of presentation likelihoods. In certain embodiments, for example, the neoantigen can be determined using one or more of the machine learning methods, software, and/or systems disclosed in the INCORPORATED REFERENCES.

Certain embodiments can provide, for example, a method for detecting antigen-binding T cells. In certain embodiments, for example, the method can comprise: isolating, from a population of PBMCs, at least one T cell that binds to a predetermined type of antigen. In certain embodiments, for example, the method can comprise: forming a plurality of cognate T cells, comprising: expanding the isolated at least one T cell. In certain embodiments, for example, the method can comprise: binding at least a first binding T cell to at least a first binding agent, comprising: contacting T cells derived from the plurality of cognate T cells with at least one of a plurality of binding agents, the at least one of the plurality of binding agents comprising the predetermined type of antigen. In certain embodiments, for example, the method can comprise: confirming that the at least a first binding T cell is configured to be activated by a cell that presents the predetermined type of antigen.

A. In certain embodiments, for example, the cell that can present the predetermined type of antigen can be an antigen presenting cell. In certain embodiments, for example, the antigen presenting cell can be a professional antigen presenting cell.

B. In certain embodiments, for example, the predetermined type of antigen can be a peptide. In certain embodiments, for example, the peptide can consist of 8-15 (for example 8-12) amino acids. In certain embodiments, for example, the peptide can consist of 12-40 amino acids. In certain embodiments, for example, the predetermined type of antigen can be derived from a tumor (for example a solid tumor). In certain embodiments, for example, the predetermined type of antigen can be presented on a tumor. In certain embodiments, for example, the predetermined type of antigen can be a personalized antigen. In certain embodiments, for example, the predetermined type of antigen can be a shared tumor antigen (for example a shared tumor neoantigen). In certain embodiments, for example, the shared tumor antigen can be a cancer/testis antigen. In certain embodiments, for example, the shared tumor antigen can be a cancer/testis-like antigen. In certain embodiments, for example, the shared tumor antigen can be a tumor associated peptide antigen. In certain embodiments, for example, the predetermined type of antigen can be characteristic of a particular type of tumor. In certain embodiments, for example, the predetermined type of antigen can be a tumor associated peptide antigen. In certain embodiments, for example, the predetermined type of antigen can be a viral antigen (for example an oncogenic viral protein such as HPV E6 and HPV E7). In certain embodiments, for example, the predetermined type of antigen can be a neoantigen. In certain embodiments, for example, the neoantigen can be a peptide. In certain embodiments, for example, the peptide can consist of 8-15 amino acids. In certain embodiments, for example, the peptide can consist of 12-40 amino acids. In certain embodiments, for example, the neoantigen can be derived from a tumor. In certain embodiments, for example, the tumor can be a solid tumor. In certain embodiments, for example, the neoantigen can be presented on a tumor. In certain embodiments, for example, the neoantigen can be a personalized neoantigen. In certain embodiments, for example, the neoantigen can be a shared tumor neoantigen. In certain embodiments, for example, the shared tumor neoantigen can be a tumor associated peptide neoantigen. In certain embodiments, for example, the neoantigen can be characteristic of a particular type of tumor. In certain embodiments, for example, the neoantigen can be a tumor associated peptide neoantigen. In certain embodiments, for example, the neoantigen can be selected from one or more neoantigens identified by a model. In certain embodiments, for example, the one or more neoantigens can be personalized neoantigens. In certain embodiments, for example, the one or more neoantigens can be present in a list of shared neoantigens. In certain embodiments, for example, the neoantigen can be selected from one or more neoantigens identified by an artificial intelligence model. In certain embodiments, for example, the model can be calibrated using machine learning. In certain embodiments, for example, the artificial intelligence model can comprise a neural network. In certain embodiments, for example, the neoantigen can be selected from a set of presentation likelihoods. In certain embodiments, for example, the neoantigen can be determined using one or more of the machine learning methods, software, and/or systems disclosed in the INCORPORATED REFERENCES.

Certain embodiments can provide, for example, a method for selection of T cell receptors specific for a predetermined type of antigen. In certain embodiments, for example, the method can comprise: isolating a first plurality of T cells, at least a portion of the first plurality of T cells bound to a plurality of P-loaded MHC proteins, which P is the predetermined type of antigen. In certain embodiments, for example, the method can comprise: further isolating a second plurality of T cells, at least a portion of the second plurality of T cells upregulating one or more activation signaling molecules (and/or expressing one or more activation markers) in the presence of a plurality of activation agents, wherein at least one of the plurality of activation agents is immunogenic for the predetermined type of antigen. In certain embodiments, for example, the method can comprise: identifying at least a portion of at least one T cell receptor sequence that is common to both the at least a portion of the first plurality of T cells and the at least a portion of the second plurality of T cells.

A. In certain embodiments, for example, the at least a portion of the at least one T cell receptor sequence can be present in at least 0.005% of the at least a portion of the first plurality of T cells and the at least a portion of the second plurality of T cells combined. In certain embodiments, for example, the at least one of the plurality of activation agents can be antigenic for the predetermined type of antigen.

B. In certain embodiments, for example, the predetermined type of antigen can be a peptide. In certain embodiments, for example, the peptide can consist of 8-15 (for example 8-12) amino acids. In certain embodiments, for example, the peptide can consist of 12-40 amino acids. In certain embodiments, for example, the predetermined type of antigen can be derived from a tumor (for example a solid tumor). In certain embodiments, for example, the predetermined type of antigen can be presented on a tumor. In certain embodiments, for example, the predetermined type of antigen can be a personalized antigen. In certain embodiments, for example, the predetermined type of antigen can be a shared tumor antigen (for example a shared tumor neoantigen). In certain embodiments, for example, the shared tumor antigen can be a cancer/testis antigen. In certain embodiments, for example, the shared tumor antigen can be a cancer/testis-like antigen. In certain embodiments, for example, the shared tumor antigen can be a tumor associated peptide antigen. In certain embodiments, for example, the predetermined type of antigen can be characteristic of a particular type of tumor. In certain embodiments, for example, the predetermined type of antigen can be a tumor associated peptide antigen. In certain embodiments, for example, the predetermined type of antigen can be a viral antigen (for example an oncogenic viral protein such as HPV E6 and HPV E7). In certain embodiments, for example, the predetermined type of antigen can be a neoantigen. In certain embodiments, for example, the neoantigen can be a peptide. In certain embodiments, for example, the peptide can consist of 8-15 amino acids. In certain embodiments, for example, the peptide can consist of 12-40 amino acids. In certain embodiments, for example, the neoantigen can be derived from a tumor. In certain embodiments, for example, the tumor can be a solid tumor. In certain embodiments, for example, the neoantigen can be presented on a tumor. In certain embodiments, for example, the neoantigen can be a personalized neoantigen. In certain embodiments, for example, the neoantigen can be a shared tumor neoantigen. In certain embodiments, for example, the shared tumor neoantigen can be a tumor associated peptide neoantigen. In certain embodiments, for example, the neoantigen can be characteristic of a particular type of tumor. In certain embodiments, for example, the neoantigen can be a tumor associated peptide neoantigen. In certain embodiments, for example, the neoantigen can be selected from one or more neoantigens identified by a model. In certain embodiments, for example, the one or more neoantigens can be personalized neoantigens. In certain embodiments, for example, the one or more neoantigens can be present in a list of shared neoantigens. In certain embodiments, for example, the neoantigen can be selected from one or more neoantigens identified by an artificial intelligence model. In certain embodiments, for example, the model can be calibrated using machine learning. In certain embodiments, for example, the artificial intelligence model can comprise a neural network. In certain embodiments, for example, the neoantigen can be selected from a set of presentation likelihoods. In certain embodiments, for example, the neoantigen can be determined using one or more of the machine learning methods, software, and/or systems disclosed in the INCORPORATED REFERENCES.

Certain embodiments can provide, for example, a method for selection of T cell receptors specific for a predetermined type of antigen. In certain embodiments, for example, the method can comprise: isolating a first plurality of T cells, at least a portion of the first plurality of T cells expressing one or more first activation markers in the presence of a plurality of first activation agents. In certain embodiments, for example, the method can comprise: further isolating a second plurality of T cells, at least a portion of the second plurality of T cells upregulating one or more second activation markers (and/or one or more activation signaling molecules) in the presence of a plurality of second activation agents. In certain embodiments, for example, the method can comprise: identifying at least one of the portion of the first plurality of T cells and at least one of the portion of the second T cells having—a) at least a portion of at least one T cell receptor sequence in common; and b) dissociation constants with a P-loaded MHC protein that are below a threshold value, which P is the predetermined type of antigen.

A. In certain embodiments, for example, at least one of the plurality of first activation agents can be immunogenic for the predetermined type of antigen, and/or at least one of the plurality of second activation agents can be immunogenic for the predetermined type of antigen. In certain embodiments, for example, at least one of the plurality of first activation agents can be antigenic for the predetermined type of antigen, and/or at least one of the plurality of second activation agents can be antigenic for the predetermined type of antigen. In certain embodiments, for example, at least one of the plurality of first activation agents can comprise the predetermined type of antigen, and/or at least one of the plurality of second activation agents can comprise the predetermined type of antigen. In certain embodiments, for example, at least one of the plurality of first activation agents can be a cell that presents the predetermined type of antigen, and/or at least one of the plurality of second activation agents can be a cell that presents the predetermined type of antigen. In certain embodiments, for example, at least one of the plurality of first activation agents can comprise P-loaded MHC protein, and/or at least one of the plurality of second activation agents P-loaded MHC protein. In certain embodiments, for example, at least one of the plurality of first activation agents can be a cell that endogenously expresses the predetermined type of antigen, and/or at least one of the plurality of second activation agents can be a cell that endogenously expresses the predetermined type of antigen. In certain embodiments, for example, at least one of the plurality of first activation agents can comprise a P-loaded MHC protein, and/or at least one of the plurality of second activation agents can be a cell that endogenously expresses the predetermined type of antigen.

B. In certain embodiments, for example, the dissociation constants can correspond to binding between the at least a portion of at least one T cell receptor sequence and the P-loaded MHC protein. In certain embodiments, for example, the threshold value can be less than 1000 μM (for example less than 50 μM).

C. In certain embodiments, for example, the predetermined type of antigen can be a peptide. In certain embodiments, for example, the peptide can consist of 8-15 (for example 8-12) amino acids. In certain embodiments, for example, the peptide can consist of 12-40 amino acids. In certain embodiments, for example, the predetermined type of antigen can be derived from a tumor (for example a solid tumor). In certain embodiments, for example, the predetermined type of antigen can be presented on a tumor. In certain embodiments, for example, the predetermined type of antigen can be a personalized antigen. In certain embodiments, for example, the predetermined type of antigen can be a shared tumor antigen (for example a shared tumor neoantigen). In certain embodiments, for example, the shared tumor antigen can be a cancer/testis antigen. In certain embodiments, for example, the shared tumor antigen can be a cancer/testis-like antigen. In certain embodiments, for example, the shared tumor antigen can be a tumor associated peptide antigen. In certain embodiments, for example, the predetermined type of antigen can be characteristic of a particular type of tumor. In certain embodiments, for example, the predetermined type of antigen can be a tumor associated peptide antigen. In certain embodiments, for example, the predetermined type of antigen can be a viral antigen (for example an oncogenic viral protein such as HPV E6 and HPV E7). In certain embodiments, for example, the predetermined type of antigen can be a neoantigen. In certain embodiments, for example, the neoantigen can be a peptide. In certain embodiments, for example, the peptide can consist of 8-15 amino acids. In certain embodiments, for example, the peptide can consist of 12-40 amino acids. In certain embodiments, for example, the neoantigen can be derived from a tumor. In certain embodiments, for example, the tumor can be a solid tumor. In certain embodiments, for example, the neoantigen can be presented on a tumor. In certain embodiments, for example, the neoantigen can be a personalized neoantigen. In certain embodiments, for example, the neoantigen can be a shared tumor neoantigen. In certain embodiments, for example, the shared tumor neoantigen can be a tumor associated peptide neoantigen. In certain embodiments, for example, the neoantigen can be characteristic of a particular type of tumor. In certain embodiments, for example, the neoantigen can be a tumor associated peptide neoantigen. In certain embodiments, for example, the neoantigen can be selected from one or more neoantigens identified by a model. In certain embodiments, for example, the one or more neoantigens can be personalized neoantigens. In certain embodiments, for example, the one or more neoantigens can be present in a list of shared neoantigens. In certain embodiments, for example, the neoantigen can be selected from one or more neoantigens identified by an artificial intelligence model. In certain embodiments, for example, the model can be calibrated using machine learning. In certain embodiments, for example, the artificial intelligence model can comprise a neural network. In certain embodiments, for example, the neoantigen can be selected from a set of presentation likelihoods. In certain embodiments, for example, the neoantigen can be determined using one or more of the machine learning methods, software, and/or systems disclosed in the INCORPORATED REFERENCES.

Certain embodiments can provide, for example, a method for negative selection of T cell receptor clonotypes. In certain embodiments, for example, the method can comprise: analyzing a mixture of T cells to identify first antigen-binding T cells and first antigen-activated T cells for a predetermined first type of antigen and second antigen-activated T cells for a predetermined second type of antigen. In certain embodiments, for example, the method can comprise: identifying at least a portion of at least one T cell receptor sequence—a) shared by at least one of the first antigen-binding T cells and at least one of the first antigen-activated T cells; and b) not shared with any of the second antigen-activated T cells.

A. In certain embodiments, for example, the predetermined first type of antigen and/or the predetermined second type of antigen can be a peptide. In certain embodiments, for example, the peptide can consist of 8-15 (for example 8-12) amino acids. In certain embodiments, for example, the peptide can consist of 12-40 amino acids. In certain embodiments, for example, the predetermined first type of antigen and/or the predetermined second type of antigen can be derived from a tumor (for example a solid tumor). In certain embodiments, for example, the predetermined first type of antigen and/or the predetermined second type of antigen can be presented on a tumor. In certain embodiments, for example, the predetermined first type of antigen and/or the predetermined second type of antigen can be a personalized antigen. In certain embodiments, for example, the predetermined first type of antigen and/or the predetermined second type of antigen can be a shared tumor antigen (for example a shared tumor neoantigen). In certain embodiments, for example, the shared tumor antigen can be a cancer/testis antigen. In certain embodiments, for example, the shared tumor antigen can be a cancer/testis-like antigen. In certain embodiments, for example, the shared tumor antigen can be a tumor associated peptide antigen. In certain embodiments, for example, the predetermined first type of antigen and/or the predetermined second type of antigen can be characteristic of a particular type of tumor. In certain embodiments, for example, the predetermined first type of antigen and/or the predetermined second type of antigen can be a tumor associated peptide antigen. In certain embodiments, for example, the predetermined first type of antigen and/or the predetermined second type of antigen can be a neoantigen. In certain embodiments, for example, the neoantigen can be a peptide. In certain embodiments, for example, the peptide can consist of 8-15 amino acids. In certain embodiments, for example, the peptide can consist of 12-40 amino acids. In certain embodiments, for example, the neoantigen can be derived from a tumor. In certain embodiments, for example, the tumor can be a solid tumor. In certain embodiments, for example, the neoantigen can be presented on a tumor. In certain embodiments, for example, the neoantigen can be a personalized neoantigen. In certain embodiments, for example, the neoantigen can be a shared tumor neoantigen. In certain embodiments, for example, the shared tumor neoantigen can be a tumor associated peptide neoantigen. In certain embodiments, for example, the neoantigen can be characteristic of a particular type of tumor. In certain embodiments, for example, the neoantigen can be a tumor associated peptide neoantigen. In certain embodiments, for example, the neoantigen can be selected from one or more neoantigens identified by a model. In certain embodiments, for example, the one or more neoantigens can be personalized neoantigens. In certain embodiments, for example, the one or more neoantigens can be present in a list of shared neoantigens. In certain embodiments, for example, the neoantigen can be selected from one or more neoantigens identified by an artificial intelligence model. In certain embodiments, for example, the model can be calibrated using machine learning. In certain embodiments, for example, the artificial intelligence model can comprise a neural network. In certain embodiments, for example, the neoantigen can be selected from a set of presentation likelihoods. In certain embodiments, for example, the neoantigen can be determined using one or more of the machine learning methods, software, and/or systems disclosed in the INCORPORATED REFERENCES. In certain embodiments, for example, the predetermined second type of antigen can not be presented on a tumor. In certain embodiments, for example, the predetermined second type of antigen can not be a personalized antigen. In certain embodiments, for example, the predetermined second type of antigen can not be a shared tumor antigen (for example a shared tumor neoantigen). In certain embodiments, for example, the shared tumor antigen can not be a cancer/testis antigen. In certain embodiments, for example, the predetermined second type of antigen can not be characteristic of a particular type of tumor. In certain embodiments, for example, the predetermined second type of antigen can not be a tumor associated peptide antigen. In certain embodiments, for example, the predetermined second type of antigen can not be a neoantigen. In certain embodiments, for example, the predetermined first type of antigen can be a first peptide and the predetermined second type of antigen can be a second peptide. In certain embodiments, for example, the first peptide can be expressed by a variant of a gene that expresses the second peptide. In certain embodiments, for example, the first peptide can be expressed by an allele of a gene that expresses the second peptide. In certain embodiments, for example, the second peptide can be expressed by a wild type gene. In certain embodiments, for example, the first peptide can be a neoantigen and the second peptide can be expressed by a related wild type gene. In certain embodiments, for example, the first peptide and the second peptide can differ by at least 1 amino acid, for example differ by at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 12 amino acids, or the first peptide and the second peptide can differ by at least 15 amino acids. In certain embodiments, for example, the first peptide and the second peptide can differ by between 1 and 15 amino acids, for example differ by between 5 and 10 amino acids, or the first peptide and the second peptide can differ by between 5 and 8 amino acids. In certain embodiments, for example, the differences can comprise (for consist) of conservative substitutions. In certain embodiments, for example, the differences can comprise (for consist) of radical substitutions. In certain embodiments, for example, the first peptide and the second peptide can have sequence identity of less than 95%, for example less than 90%, less than 85%, less than 80%, less than 75%, less than 70%, less than 65%, less than 60%, or the first peptide and the second peptide can have sequence identity of less than 55%. In certain embodiments, for example, the first peptide and the second peptide can have sequence identity of between 55% and 95%, for example between 55% and 90%, between 55% and 85%, between 55% and 80%, between 55% and 75%, or the first peptide and the second peptide can have sequence identity of between 55% and 70%.

B. In certain embodiments, for example, identifying the first antigen-activated T cells can comprise contacting a portion of the mixture of T cells with cells that endogenously present (for example by expression, such as a tumor cell) the predetermined first type of antigen. In certain embodiments, for example, identifying the second antigen-activated T cells can comprise contacting a portion of the mixture of T cells with cells that endogenously present (for example by expression, such as a tumor cell) the predetermined second type of antigen. In certain embodiments, for example, identifying the first antigen-activated T cells can comprise contacting a portion of the mixture of T cells with cells that have been loaded (for example from an exogenous source, such as professional antigen presenting cells) with the predetermined first type of antigen. In certain embodiments, for example, identifying the second antigen-activated T cells can comprise contacting a portion of the mixture of T cells with cells that have been loaded (for example from an exogenous source, such as professional antigen presenting cells) with the predetermined second type of antigen.

Certain embodiments can provide, for example, a method for negative selection of T cell receptor clonotypes. In certain embodiments, for example, the method can comprise: analyzing a mixture of T cells to identify first antigen-activated T cells and first antigen-binding T cells for a predetermined first type of antigen and second antigen-binding T cells for a predetermined second type of antigen. In certain embodiments, for example, the method can comprise: identifying at least a portion of at least one T cell receptor sequence—a) shared by at least one of the first antigen-binding T cells and at least one of the first antigen-activated T cells; and b) not shared with any of the second antigen-binding T cells.

Certain embodiments can provide, for example, a method for identifying a T cell activation marker. In certain embodiments, for example, the method can comprise: contacting a first plurality of T cells with a plurality of P-presenting (for examples cells that express P or cells that have been pulsed with P) cells (for example a plurality of P-presenting cells comprising P presented via MHC Class I proteins and/or MHC Class II proteins), the first plurality of T cells comprising a plurality of P-binding T cells, which P is a predetermined type of antigen. In certain embodiments, for example, the method can comprise: measuring a plurality of expression rate profiles for at least a portion of the contacted plurality of P-binding T cells. In certain embodiments, for example, the method can comprise: measuring a functional response to P in at least two T cells present in the at least a portion of the contacted plurality of P-binding T cells. In certain embodiments, for example, the method can comprise: partitioning, into a plurality of T cell clusters, the at least a portion of the contacted plurality of P-binding T cells. In certain embodiments, for example, the method can comprise: mapping the expression rate profiles to the plurality of T cell clusters to identify one of the plurality of T cell clusters comprising the at least two T cells. In certain embodiments, for example, the method can comprise: identifying an activation marker (or secreted molecule indicative of activation) that is expressed by the at least two T cells.

A. In certain embodiments, for example, the P-binding T cells can be identified using a bioinformatics filter that compares at least portions of T cell receptor sequences of the at least a portion of the contacted plurality of P-binding T cells with at least portions of predetermined T cell receptor sequences.

B. In certain embodiments, for example, the partitioning can comprise: partitioning the contacted plurality of P-binding T cells into groups, at least one of the groups consisting of T cells having at least portions of T cell receptor sequences in common. In certain embodiments, for example, the partitioning can comprise: partitioning the contacted plurality of P-binding T cells into groups, at least one of the groups consisting of T cells having at least portions of T cell receptor sequences characterized by sequence identities of at least 70% (for example at least 80%, 90%, 95%, 100%) to one another.

In certain embodiments, for example, the partitioning can comprise: partitioning the contacted plurality of P-binding T cells into groups, at least one of the groups consisting of T cells having at least portions of T cell receptor sequences that differ by at most 1 amino acid (for example a conservative substitution of at most 1 amino acid) between one another. In certain embodiments, for example, the partitioning can comprise: partitioning the contacted plurality of P-binding T cells into groups, at least one of the groups consisting of T cells having at least portions of T cell receptor sequences that differ by only conservative substitutions. In certain embodiments, for example, the partitioning can comprise grouping of lymphocyte interactions by paratope hotspots (GLIPH). In certain embodiments, for example, the at least portions of T cell receptor sequences in common can be at least portions of a CDR3 region. In certain embodiments, for example, the at least portions of the CDR3 region can comprise a linear amino acid sequences having lengths of between 6 and 35 amino acids. In certain embodiments, for example, the at least portions of the CDR3 region can be exclusive of stem regions. In certain embodiments, for example, the at least portions of the CDR3 region can comprise CDR3 beta chain portions.

C. In certain embodiments, for example, the partitioning can be performed using an algorithm (for example a statistical algorithm). In certain embodiments, for example, the algorithm can comprise a similarity analysis of the plurality of expression rate profiles. In certain embodiments, for example, the plurality of expression rate profiles can comprise expression rates for one or more activation markers indicative of a functional response to P. In certain embodiments, for example, the one or more activation markers can compromise CD137, CD69, CD25, Ki67, CD107, CD122, CD27, CD28, CD95, CD134, KLRG1, CD38, CD154, or a combination of two or more of the foregoing activation markers. In certain embodiments, for example, the algorithm can be a cluster analysis algorithm. In certain embodiments, for example, the algorithm can comprise t-distributed stochastic neighbor embedding.

D. In certain embodiments, for example, the measured functional response to P can comprise detection of one or more activation markers and/or one or more secreted molecules. In certain embodiments, for example, the one or more activation markers can compromise CD137, CD69, CD25, Ki67, CD107, CD122, CD27, CD28, CD95, CD134, KLRG1, CD38, CD154, or a combination of two or more of the foregoing activation markers. In certain embodiments, for example, the one or more secreted molecules can comprise one or more cytokines. In certain embodiments, for example, the one or more cytokines can be interferon gamma (IFN-gamma), tumor necrosis factor alpha (TNFalpha), interleukin-2 (IL-2), or a combination of two or more of the foregoing. In certain embodiments, for example, the one or more secreted molecules can comprise granzyme. In certain embodiments, for example, the one or more secreted molecules can comprise perforin. In certain embodiments, for example, the measured functional response to P can comprise detection of T cell proliferation.

E. In certain embodiments, for example, the first plurality of T cells and the second plurality of T cells can be derived from a common starting population of PBMCs.

F. In certain embodiments, for example, the plurality of expression rate profiles can be obtained from a series of single-cell transcriptome analyses. In certain embodiments, for example, T cells in the one of the plurality of T cell clusters can express the predetermined first activation marker at an average second expression rate that exceeds a first expression rate threshold (for example a first expression rate threshold of greater than 0.05% (for example greater than 0.1%, greater than 0.5%, or between 0.05% and 0.5%)). In certain embodiments, for example, T cells in the one of the plurality of T cell clusters can express the second activation marker at an average second expression rate that exceeds a second expression rate threshold (for example a second expression rate threshold of greater than 0.05% (for example greater than 0.1%, greater than 0.5%, or between 0.05% and 0.5%)).

G. In certain embodiments, for example, the method can further comprise identifying the plurality of P-binding T cells by matching T cell receptor sequences of the plurality of P-binding T cells to predetermined T cell receptor sequences. In certain embodiments, for example, the predetermined T cell receptor sequences can be determined by sequencing a second plurality of T cells bound to P-loaded MHC proteins.

In certain embodiments, for example, the activation marker can not be expressed or can be downregulated in at least two other T cells present in another one of the plurality of T cell clusters, wherein the at least two other T cells do not show a functional response when measured.

H. In certain embodiments, for example, the predetermined type of antigen can be a peptide. In certain embodiments, for example, the peptide can consist of 8-15 (for example 8-12) amino acids. In certain embodiments, for example, the peptide can consist of 12-40 amino acids. In certain embodiments, for example, the predetermined type of antigen can be derived from a tumor (for example a solid tumor). In certain embodiments, for example, the predetermined type of antigen can be presented on a tumor. In certain embodiments, for example, the predetermined type of antigen can be a personalized antigen. In certain embodiments, for example, the predetermined type of antigen can be a shared tumor antigen (for example a shared tumor neoantigen). In certain embodiments, for example, the shared tumor antigen can be a cancer/testis antigen. In certain embodiments, for example, the shared tumor antigen can be a cancer/testis-like antigen. In certain embodiments, for example, the shared tumor antigen can be a tumor associated peptide antigen. In certain embodiments, for example, the predetermined type of antigen can be characteristic of a particular type of tumor. In certain embodiments, for example, the predetermined type of antigen can be a tumor associated peptide antigen. In certain embodiments, for example, the predetermined type of antigen can be a viral antigen (for example an oncogenic viral protein such as HPV E6 and HPV E7). In certain embodiments, for example, the predetermined type of antigen can be a neoantigen. In certain embodiments, for example, the neoantigen can be a peptide. In certain embodiments, for example, the peptide can consist of 8-15 amino acids. In certain embodiments, for example, the peptide can consist of 12-40 amino acids. In certain embodiments, for example, the neoantigen can be derived from a tumor. In certain embodiments, for example, the tumor can be a solid tumor. In certain embodiments, for example, the neoantigen can be presented on a tumor. In certain embodiments, for example, the neoantigen can be a personalized neoantigen. In certain embodiments, for example, the neoantigen can be a shared tumor neoantigen. In certain embodiments, for example, the shared tumor neoantigen can be a tumor associated peptide neoantigen. In certain embodiments, for example, the neoantigen can be characteristic of a particular type of tumor. In certain embodiments, for example, the neoantigen can be a tumor associated peptide neoantigen. In certain embodiments, for example, the neoantigen can be selected from one or more neoantigens identified by a model. In certain embodiments, for example, the one or more neoantigens can be personalized neoantigens. In certain embodiments, for example, the one or more neoantigens can be present in a list of shared neoantigens. In certain embodiments, for example, the neoantigen can be selected from one or more neoantigens identified by an artificial intelligence model. In certain embodiments, for example, the model can be calibrated using machine learning. In certain embodiments, for example, the artificial intelligence model can comprise a neural network. In certain embodiments, for example, the neoantigen can be selected from a set of presentation likelihoods. In certain embodiments, for example, the neoantigen can be determined using one or more of the machine learning methods, software, and/or systems disclosed in the INCORPORATED REFERENCES.

Certain embodiments can provide, for example, a method for prequalifying T cell receptors for development of therapeutically effective antigen-binding and antigen-activated T cells. In certain embodiments, for example, the development can comprise transfecting the T cell receptors into T cell lines. In certain embodiments, for example, the T cell receptors can be identified from T cells derived from one or more healthy HLA-matched donor samples. In certain embodiments, for example, the identified T cell receptors can be present in less than 1 in 10,000 of the derived T cells. In certain embodiments, for example, the derived T cells can be obtained by one or more of the foregoing enriching and/or expanding steps described herein or in one of the INCORPORATED REFERENCES. In certain embodiments, for example, the identified T cell receptors can be present in less than 1 in 10,000,000 of the T cells present in the one more donor samples. In certain embodiments, for example, antigen-activation of at least a portion of the derived T cells can be determined by exposing to cells presenting the antigen at a physiological concentration (for example a concentration in a range in which the antigen would be presented on a tumor cell).

Certain embodiments can provide, for example, a method for selecting T cell receptors. In certain embodiments, for example, the method can comprise expanding (for example by polyclonal expansion) a series of anti-antigen T cells. In certain embodiments, for example, the method can comprise obtaining a T cell from one of the series of anti-antigen T cells, followed by complexing the obtained anti-antigen T cell with a binding agent (for example with a fluorescently labeled antigen-MHC protein multimer or a magnetically tagged antigen-MHC protein multimer), and then expanding the complexed anti-antigen T cell to form a member of the next series of anti-antigen T cells. In certain embodiments, for example, the method can further comprise exposing a cognate of the member to a cell expressing the antigen at a physiologically relevant concentration, followed by detecting activation of the cognate. Certain embodiments can provide, for example, a therapy for cancer, comprising administering a T cell, the T cell comprising a transfected T cell receptor, the T cell receptor comprising at least a portion of a T cell receptor sequence that was determined by sequencing the cognate.

Certain embodiments can provide, for example, a method for screening a candidate antigen for an antigen-specific vaccine. In certain embodiments, for example, the method can comprise: isolating, from a population of PBMCs, at least one T cell that binds to the candidate antigen. In certain embodiments, for example, the method can comprise: forming a plurality of cognate T cells, comprising: expanding the isolated at least one T cell. In certain embodiments, for example, the method can comprise: activating at least a first functional T cell, comprising: contacting T cells derived from the plurality of cognate T cells with at least one of a plurality of activation agents that is immunogenic for the candidate antigen.

A. In certain embodiments, for example, the candidate antigen can be a neoantigen. In certain embodiments, for example, the antigen-specific vaccine can be for treatment of a cancer (for example for treatment of a cancerous tumor).

Certain embodiments can provide, for example, a method for screening candidate neoantigen for immunogenicity. In certain embodiments, for example, the method can comprise: isolating, from a population of PBMCs, at least one T cell that binds to the candidate neoantigen. In certain embodiments, for example, the method can comprise: forming a plurality of cognate T cells, comprising: expanding the isolated at least one T cell. In certain embodiments, for example, the method can comprise: activating at least a first functional T cell, comprising: contacting T cells derived from the plurality of cognate T cells with at least one of a plurality of activation agents that is immunogenic for the candidate neoantigen.

Certain embodiments can provide, for example, an artificial T cell receptor selective to a predetermined type of antigen (for example a neoantigen such as a personal neoantigen or a shared neoantigen). In certain embodiments, for example, the method can comprise: at least a portion of a CDR3 region selected by—a) analyzing a mixture of natural T cells to identify antigen-binding T cells and antigen-activated T cells for the predetermined type of antigen; and b) identifying at least a portion of at least one T cell receptor sequence shared by at least one of the antigen-binding T cells and at least one of the antigen-activated T cells, the at least a portion of at least one T cell receptor sequence containing the at least a portion of the CDR3 region. In certain embodiments, for example, the method can comprise: a T cell receptor fragment (for example a universal backbone fragment that can be combined with a plurality of different CDR3 sequences to form a plurality of products, for example a plurality of therapeutic products).

Certain embodiments can provide, for example, a method for selection of T cell receptor clonotypes, comprising: i) analyzing a mixture of T cells to identify antigen-binding T cells and antigen-activated T cells for a predetermined type of antigen; and ii) identifying at least a portion of at least one T cell receptor sequence shared by at least one of the antigen-binding T cells and at least one of the antigen-activated T cells.

Certain embodiments can provide, for example, a method for selection of shared receptor sequences in lymphocytes, comprising: i) analyzing a mixture of lymphocytes to identify stimulated lymphocytes and costimulated lymphocytes for a predetermined type of antigen; and ii) identifying at least a portion of at least one receptor sequence shared by at least one of the stimulated lymphocytes and at least one of the costimulated lymphocytes.

Certain embodiments can provide, for example, a method for selection of T cell receptor clonotypes, comprising: i) analyzing a mixture of naïve T cells to identify antigen-binding T cells and functional T cells for a predetermined type of antigen; and ii) identifying at least a portion of at least one T cell receptor sequence shared by at least one of the antigen-binding T cells and at least one of the functional T cells.

Certain embodiments can provide, for example, a method for selection of T cell receptors, comprising: i) binding at least a first antigen-binding T cell to at least a first one of a predetermined type of antigen, comprising: contacting a first plurality of T cells with the first one of the predetermined type of antigen; ii) activating at least a first functional T cell, comprising: contacting a second plurality of T cells with a plurality of cells that present at least a second one of the predetermined type of antigen; and iii) identifying at least a portion of at least one T cell receptor sequence that is common to the at least one antigen-binding T cell and the at least one functional T cell.

Certain embodiments can provide, for example, a method for selection of T cell receptors, comprising: i) binding at least a first antigen-binding T cell present in a first plurality of T cells to at least a first one of a Class I P-MHC protein multimer, which P is a predetermined type of antigen, comprising: contacting the first plurality of T cells with the first one of the Class I P-MHC protein multimer; ii) activating at least a first functional T cell present in a second plurality of T cells, comprising: contacting the second plurality of T cells with a plurality of cells that present at least a first one of a Class II P-MHC protein multimer; and iii) identifying at least a portion of at least one T cell receptor sequence that is common to the at least one antigen-binding T cell and the at least one functional T cell.

Certain embodiments can provide, for example, a method for selection of T cell receptors, comprising: i) binding at least a first antigen-binding T cell present in a first plurality of T cells to at least a first one of a Class I P-MHC protein multimer, which P is a predetermined type of antigen, comprising: contacting the first plurality of T cells with the first one of the Class I P-MHC protein multimer; ii) activating at least a first functional T cell present in a second plurality of T cells, comprising: contacting the second plurality of T cells with a plurality of cells that present at least a first Class I P-MHC protein; and iii) identifying at least a portion of at least one T cell receptor sequence that is common to the at least one antigen-binding T cell and the at least one functional T cell.

Certain embodiments can provide, for example, a method for selection of T cell receptors, comprising: i) isolating a first T cell from a plurality of T cells, the first T cell bound to a P-loaded MHC protein, which P is a predetermined type of antigen; ii) further isolating a second T cell from the plurality of T cells, the second T cell expressing at least one biomarker indicative of activation by the predetermined type of antigen; and iii) matching at least a portion of a T cell receptor sequence of the first T cell with at least a portion of a T cell receptor sequence of the second T cell.

Certain embodiments can provide, for example, a method for detecting functional T cell receptor clonotypes, comprising: i) isolating, from a population of PBMCs, at least one T cell that binds to a predetermined type of antigen; ii) forming a plurality of cognate T cells, comprising: expanding the isolated at least one T cell; iii) activating at least a first functional T cell, comprising: contacting T cells derived from the plurality of cognate T cells with at least one of a plurality of activation agents that is immunogenic for the predetermined type of antigen; and iv) confirming that the at least a first functional T cell is configured to bind to a P-loaded MHC protein, which P is the predetermined type of antigen.

Certain embodiments can provide, for example, a method for detecting antigen-binding T cells, comprising: i) isolating, from a population of PBMCs, at least one T cell that binds to a predetermined type of antigen; ii) forming a plurality of cognate T cells, comprising: expanding the isolated at least one T cell; iii) binding at least a first binding T cell to at least a first binding agent, comprising: contacting T cells derived from the plurality of cognate T cells with at least one of a plurality of binding agents, the at least one of the plurality of binding agents comprising the predetermined type of antigen; and iv) confirming that the at least a first binding T cell is configured to be activated by a cell that presents the predetermined type of antigen.

Certain embodiments can provide, for example, a method for selection of T cell receptors specific for a predetermined type of antigen, comprising: i) isolating a first plurality of T cells, at least a portion of the first plurality of T cells bound to a plurality of P-loaded MHC proteins, which P is the predetermined type of antigen; ii) further isolating a second plurality of T cells, at least a portion of the second plurality of T cells upregulating one or more activation signaling molecules (and/or expressing one or more activation markers or another indicator of activation) in the presence of a plurality of activation agents, wherein at least one of the plurality of activation agents is immunogenic for the predetermined type of antigen; and iii) identifying at least a portion of at least one T cell receptor sequence that is common to both the at least a portion of the first plurality of T cells and the at least a portion of the second plurality of T cells.

Certain embodiments can provide, for example, a method for selection of T cell receptors specific for a predetermined type of antigen, comprising: i) isolating a first plurality of T cells, at least a portion of the first plurality of T cells expressing one or more first activation markers in the presence of a plurality of first activation agents; ii) further isolating a second plurality of T cells, at least a portion of the second plurality of T cells upregulating one or more second activation markers (and/or one or more activation signaling molecules) in the presence of a plurality of second activation agents; and iii) identifying at least one of the portion of the first plurality of T cells and at least one of the portion of the second T cells having—a) at least a portion of at least one T cell receptor sequence in common; and b) dissociation constants with a P-loaded MHC protein that are below a threshold value, which P is the predetermined type of antigen.

Certain embodiments can provide, for example, a method for negative selection of T cell receptor clonotypes, comprising: i) analyzing a mixture of T cells to identify first antigen-binding T cells and first antigen-activated T cells for a predetermined first type of antigen and second antigen-activated T cells for a predetermined second type of antigen; and ii) identifying at least a portion of at least one T cell receptor sequence—a) shared by at least one of the first antigen-binding T cells and at least one of the first antigen-activated T cells; and b) not shared with any of the second antigen-activated T cells.

Certain embodiments can provide, for example, a method for negative selection of T cell receptor clonotypes, comprising: i) analyzing a mixture of T cells to identify first antigen-activated T cells and first antigen-binding T cells for a predetermined first type of antigen and second antigen-binding T cells for a predetermined second type of antigen; and ii) identifying at least a portion of at least one T cell receptor sequence—a) shared by at least one of the first antigen-binding T cells and at least one of the first antigen-activated T cells; and b) not shared with any of the second antigen-binding T cells.

Certain embodiments can provide, for example, a method for identifying a T cell activation marker, comprising: i) contacting a first plurality of T cells with a plurality of P-presenting cells, the first plurality of T cells comprising a plurality of P-binding T cells, which P is a predetermined type of antigen; ii) measuring a plurality of expression rate profiles for at least a portion of the contacted plurality of P-binding T cells; iii) measuring a functional response to the predetermined type of antigen in at least two T cells present in the at least a portion of the contacted plurality of P-binding T cells; iv) partitioning, into a plurality of T cell clusters, the at least a portion of the contacted plurality of P-binding T cells; v) mapping the expression rate profiles to the plurality of T cell clusters to identify one of the plurality of T cell clusters comprising the at least two T cells; and vi) identifying an activation marker that is expressed by the at least two T cells.

Certain embodiments can provide, for example, a method for screening a candidate antigen for an antigen-specific vaccine, comprising: i) isolating, from a population of PBMCs, at least one T cell that binds to the candidate antigen; ii) forming a plurality of cognate T cells, comprising: expanding the isolated at least one T cell; and iii) activating at least a first functional T cell, comprising: contacting T cells derived from the plurality of cognate T cells with at least one of a plurality of activation agents that is immunogenic for the candidate antigen.

Certain embodiments can provide, for example, a method for screening candidate neoantigen for immunogenicity, comprising: i) isolating, from a population of PBMCs, at least one T cell that binds to the candidate neoantigen; ii) forming a plurality of cognate T cells, comprising: expanding the isolated at least one T cell; and iii) activating at least a first functional T cell, comprising: contacting T cells derived from the plurality of cognate T cells with at least one of a plurality of activation agents that is immunogenic for the candidate neoantigen.

Certain embodiments can provide, for example, an artificial T cell receptor selective to a predetermined type of antigen (for example a neoantigen such as a personal neoantigen or a shared neoantigen), comprising: i) at least a portion of a CDR3 region (for example at least a portion of a CDR3 beta chain) selected by—a) analyzing a mixture of natural T cells to identify antigen-binding T cells and antigen-activated T cells for the predetermined type of antigen; and b) identifying at least a portion of at least one T cell receptor sequence shared by at least one of the antigen-binding T cells and at least one of the antigen-activated T cells, the at least a portion of at least one T cell receptor sequence containing the at least a portion of the CDR3 region; and ii) a T cell receptor fragment (for example a universal backbone fragment that can be combined with a plurality of different CDR3 sequences to form a plurality of products, for example a plurality of therapeutic products).

Certain embodiments can provide, for example, a method for identification of one or more viral epitopes, comprising: one or more of the T cell receptor (or T cell receptor clonotypes or shared receptor sequences) identification and/or selection methods disclosed herein.

Certain embodiments can provide, for example, a composition for organ transplant therapy, comprising: one or more at least a portion of at least one T cell receptor sequence determined from one or more of the T cell receptor (or T cell receptor clonotypes or shared receptor sequences) identification and/or selection methods disclosed herein.

Certain embodiments can provide, for example, a composition for cell therapy in one or more subjects, comprising: one or more at least a portion of at least one T cell receptor sequence determined from one or more of the T cell receptor (or T cell receptor clonotypes or shared receptor sequences) identification and/or selection methods disclosed herein.

Certain embodiments can provide, for example, a composition to enhance the immune system in one or more subjects, comprising: one or more at least a portion of at least one T cell receptor sequence determined from one or more of the T cell receptor (or T cell receptor clonotypes or shared receptor sequences) identification and/or selection methods disclosed herein.

In any of the foregoing embodiments, the predetermined type of antigen can be a neoantigen (for example a neoantigen identified using a machine learning-based model).

Also provided herein, is a composition obtained by any of the methods described herein.

The present disclosure also provides a composition comprising an artificial T cell receptor selective to a predetermined type of antigen, comprising: at least a portion of a CDR3 region selected by—analyzing a mixture of natural T cells to identify antigen-binding T cells and antigen-activated T cells for the predetermined type of antigen; and identifying at least a portion of at least one T cell receptor sequence shared by at least one of the antigen-binding T cells and at least one of the antigen-activated T cells, the at least a portion of at least one T cell receptor sequence containing the at least a portion of the CDR3 region; and a T cell receptor fragment.

Certain embodiments provide a T cell comprising an artificial T cell receptor or fragment thereof obtained by any one of the methods described herein. In some embodiments, the T cell is for use in the treatment of cancer.

Also provided herein, is a kit that includes a composition obtained by any of the methods described herein.

The present disclosure also provides, a kit for use in any one of the methods provided herein.

### BRIEF DESCRIPTION OF THE DRAWINGS

FIG. 1: A schematic illustration of an approach for selection of T cell receptors.

FIG. 2: A schematic illustration of an approach for selection of T cell receptors that includes a negative selection step.

FIG. 3: A schematic illustration of an approach for identification of T cell receptor activation markers.

FIG. 4: T cell receptor sequence frequencies for ASSLPTTMNY-specific T cells (“ASSLPTTMNY” disclosed as SEQ ID NO: 1) in Example 1: CD137⁺ T cells (Y-axis) versus antigen-binding T cells (X-axis) with shared T cell receptor sequences noted. “A” denotes Reference A, described in Table 2.

FIG. 5: T cell receptor sequence frequencies for ASSLPTTMNY-specific T cells (“ASSLPTTMNY” disclosed as SEQ ID NO: 1) in Example 1: interferon gamma⁺ T cells (Y-axis) versus antigen-binding T cells (X-axis) with shared T cell receptor sequences noted. “A” denotes Reference A, described in Table 2.

FIG. 6: T cell receptor sequence frequencies for ASSLPTTMNY-specific T cells (“ASSLPTTMNY” disclosed as SEQ ID NO: 1) in duplicate of Example 1: CD137⁺ T cells (Y-axis) versus antigen-binding T cells (X-axis) with shared T cell receptor sequences noted. “B” denotes Reference B, described in Table 2.

FIG. 7: T cell receptor sequence frequencies for ASSLPTTMNY-specific T cells (“ASSLPTTMNY” disclosed as SEQ ID NO: 1) in duplicate of Example 1: interferon gamma⁺ T cells (Y-axis) versus antigen-binding T cells (X-axis) with shared T cell receptor sequences noted. “B” denotes Reference B, described in Table 2.

FIG. 8: T cell receptor sequence frequencies for HSEVGLPVY-specific T cells (“HSEVGLPVY” disclosed as SEQ ID NO: 2) in first of three activation marker measurements: CD137⁺ T cells (Y-axis) versus antigen-binding T cells (X-axis) with shared T cell receptor sequences noted. “I,” “J”, “K”, “L,” “N,” and “0” denote References I, J, K, L, N, and O, respectively, described in Table 3.

FIG. 9: T cell receptor sequence frequencies for HSEVGLPVY-specific T cells (“HSEVGLPVY” disclosed as SEQ ID NO: 2) in second of three activation marker measurements: CD137⁺ T cells (Y-axis) versus antigen-binding T cells (X-axis) with shared T cell receptor sequences noted. “J”, “L,” “M,” “N,” and “0” denote References J, L, M, N, and O, respectively, described in Table 3.

### DETAILED DESCRIPTION OF THE INVENTION

The present disclosure is based, generally, on the discovery that T cell receptors suitable for developing T cell lines for immunotherapy can be identified more quickly and with a reduced number of steps by partitioning a mixture of T cells (for example a mixture of naïve T cells derived from a starting PBMC sample) into portions that are separately tested for antigen-binding and functionality. T cell receptors from each portion can be sequenced and overlapping T cell receptors, based on both antigen-binding and functional T cells, identified for further development. The present disclosure is further specifically based, in part, on the discovery that this approach does not necessarily require in vitro priming of the starting sample, and therefore can reduce deleterious effects due to downregulation of T cell receptors and/or exposure to high concentrations of antigen. Moreover, the functional testing can be performed by exposing T cells to activation agents (for example antigen presenting cells or tumor cells) that present antigen at physiological concentrations, and are therefore more likely to identify T cell receptors that will yield functional T cell lines in practice.

T cell receptors are highly diverse heterodimers, consisting of a combination of alpha (“α”) and beta (“β”) chains (αβ TCR), or gamma delta (“γδ”) chains (γδ TCR). The T cell receptor chains consist of a variable region, important for antigen recognition, and a constant region. The variable region of T cell receptor α and δ chains is encoded by a number of variable (V) and joining (J) genes, while T cell receptor β and γ chains are additionally encoded by diversity (D) genes. Each TCR chain contains three hypervariable loops in its structure, termed complementarity determining regions (CDR1-3). CDR1 and 2 are encoded by V genes and are required for interaction of the TCR with the MHC complex. CDR3, however, is encoded by the junctional region between the V and J or D and J genes and is therefore highly variable. It plays an essential role in the interaction of the TCR with the peptide-MHC complex, as it is the region of the TCR in direct contact with the peptide antigen. For this reason, CDR3 is often used as the region of interest to determine T cell receptor clonotypes, as it is highly unlikely that two T cells will express the same CDR3 nucleotide sequence, unless they have derived from the same clonally expanded T cell.

According, certain embodiments can provide, for example, methods, compositions, assays, systems, devices and/or kits for identifying at least one T cell receptor component of antigen-binding and antigen-activated T cells for a predetermined type of antigen. For example, in some embodiments, the present disclosure provides methods for selection of T cell receptor clonotypes.

In certain embodiments, the method for selection of T cell receptor clonotypes includes analyzing a mixture of T cells to identify antigen-binding T cells and antigen-activated T cells for a predetermined type of antigen; and identifying at least a portion of at least one T cell receptor sequence shared by at least one of the antigen-binding T cells and at least one of the antigen-activated T cells. In some embodiments, the method for selection of T cell receptor clonotypes includes analyzing a mixture of naïve T cells to identify antigen-binding T cells and functional T cells for a predetermined type of antigen; and identifying at least a portion of at least one T cell receptor sequence shared by at least one of the antigen-binding T cells and at least one of the functional T cells.

Also provided herein is a method for selection of shared receptor sequences in lymphocytes that includes analyzing a mixture of lymphocytes to identify stimulated lymphocytes and costimulated lymphocytes for a predetermined type of antigen; and identifying at least a portion of at least one receptor sequence shared by at least one of the stimulated lymphocytes and at least one of the costimulated lymphocytes.

The present disclosure also provides methods for selection of T cell receptors. In some embodiments, the method for selection of T cell receptors includes binding at least a first antigen-binding T cell to at least a first one of a predetermined type of antigen, including: contacting a first plurality of T cells with the first one of the predetermined type of antigen; activating at least a first functional T cell, including: contacting a second plurality of T cells with a plurality of cells that present at least a second one of the predetermined type of antigen; and identifying at least a portion of at least one T cell receptor sequence that is common to the at least one antigen-binding T cell and the at least one functional T cell.

In other embodiments, the method for selection of T cell receptors includes binding at least a first antigen-binding T cell present in a first plurality of T cells to at least a first one of a Class I P-MHC protein multimer, which P is a predetermined type of antigen, comprising: contacting the first plurality of T cells with the first one of the Class I P-MHC protein multimer; activating at least a first functional T cell present in a second plurality of T cells, comprising: contacting the second plurality of T cells with a plurality of cells that present at least a first one of a Class II P-MHC protein multimer; and identifying at least a portion of at least one T cell receptor sequence that is common to the at least one antigen-binding T cell and the at least one functional T cell. In yet other embodiments, the method for selection of T cell receptors includes binding at least a first antigen-binding T cell present in a first plurality of T cells to at least a first one of a Class II P-MHC protein multimer, which P is a predetermined type of antigen, comprising: contacting the first plurality of T cells with the first one of the Class II P-MHC protein multimer; activating at least a first functional T cell present in a second plurality of T cells, comprising: contacting the second plurality of T cells with a plurality of cells that present at least a first Class I P-MHC protein; and identifying at least a portion of at least one T cell receptor sequence that is common to the at least one antigen-binding T cell and the at least one functional T cell.

In some embodiments, the method for selection of T cell receptors includes binding at least a first antigen-binding T cell present in a first plurality of T cells to at least a first one of a Class I P-MHC protein multimer, where P is a predetermined type of antigen, that includes: contacting the first plurality of T cells with the first one of the Class I P-MHC protein multimer; activating at least a first functional T cell present in a second plurality of T cells, comprising: contacting the second plurality of T cells with a plurality of cells that present at least a first Class I P-MHC protein; and identifying at least a portion of at least one T cell receptor sequence that is common to the at least one antigen-binding T cell and the at least one functional T cell. In other embodiments, the method for selection of T cell receptors includes binding at least a first antigen-binding T cell present in a first plurality of T cells to at least a first one of a Class II P-MHC protein multimer, where P is a predetermined type of antigen, that includes: contacting the first plurality of T cells with the first one of the Class II P-MHC protein multimer; activating at least a first functional T cell present in a second plurality of T cells, comprising: contacting the second plurality of T cells with a plurality of cells that present at least a first one of a Class II P-MHC protein multimer; and identifying at least a portion of at least one T cell receptor sequence that is common to the at least one antigen-binding T cell and the at least one functional T cell.

In yet another embodiment, method for selection of T cell receptors, includes isolating a first T cell from a plurality of T cells, the first T cell bound to a P-loaded MHC protein, which P is a predetermined type of antigen; further isolating a second T cell from the plurality of T cells, the second T cell expressing at least one biomarker indicative of activation by the predetermined type of antigen; and matching at least a portion of a T cell receptor sequence of the first T cell with at least a portion of a T cell receptor sequence of the second T cell.

Also provided herein, is a method for detecting antigen-binding T cells. In some embodiments, the method for detecting antigen-binding T cells includes isolating, from a population of PBMCs, at least one T cell that binds to a predetermined type of antigen; forming a plurality of cognate T cells, including: expanding the isolated at least one T cell; binding at least a first binding T cell to at least a first binding agent, comprising: contacting T cells derived from the plurality of cognate T cells with at least one of a plurality of binding agents, the at least one of the plurality of binding agents comprising the predetermined type of antigen; and confirming that the at least a first binding T cell is configured to be activated by a cell that presents the predetermined type of antigen. Antigen-binding T cells can be detected by assay with binding agents as disclosed herein or in one of the INCORPORATED REFERENCES.

The present disclosure also provides a method for detecting functional T cell receptor clonotypes, that includes isolating, from a population of PBMCs, at least one T cell that binds to a predetermined type of antigen; forming a plurality of cognate T cells, comprising: expanding the isolated at least one T cell; activating at least a first functional T cell, comprising: contacting T cells derived from the plurality of cognate T cells with at least one of a plurality of activation agents that is immunogenic for the predetermined type of antigen; and confirming that the at least a first functional T cell is configured to bind to a P-loaded MHC protein, which P is the predetermined type of antigen.

It is understood that the present disclosure also provides for methods that combine one or more of the methods described above. For example, a method for detecting functional T cell receptor clonotypes can be combined with the method for detecting antigen-binding T cells to facilitate the selection of T cell receptors that bind a specific antigen and are functional. Accordingly, in some embodiments, provided herein is a method for selection of T cell receptors specific for a predetermined type of antigen, that includes isolating a first plurality of T cells, at least a portion of the first plurality of T cells bound to a plurality of P-loaded MHC proteins, which P is the predetermined type of antigen; further isolating a second plurality of T cells, at least a portion of the second plurality of T cells upregulating one or more activation signaling molecules and/or expressing one or more activation markers in the presence of a plurality of activation agents, wherein at least one of the plurality of activation agents is immunogenic for the predetermined type of antigen; and identifying at least a portion of at least one T cell receptor sequence that is common to both the at least a portion of the first plurality of T cells and the at least a portion of the second plurality of T cells.

In other embodiments, the method for selection of T cell receptors specific for a predetermined type of antigen, includes isolating a first plurality of T cells, at least a portion of the first plurality of T cells expressing one or more first activation markers in the presence of a plurality of first activation agents; further isolating a second plurality of T cells, at least a portion of the second plurality of T cells upregulating one or more second activation markers and/or expressing one or more activation signaling molecules in the presence of a plurality of second activation agents; and identifying at least one of the portion of the first plurality of T cells and at least one of the portion of the second T cells having at least a portion of at least one T cell receptor sequence in common; and dissociation constants with a P-loaded MHC protein that are below a threshold value, which P is the predetermined type of antigen.

Accordingly, in certain embodiments, for example, the at least one T cell receptor component can comprise at least one T cell receptor component. In certain embodiments, for example, the at least one T cell receptor component can comprise at least one T cell receptor clonotype. In certain embodiments, for example, the at least one T cell receptor component can comprise at least one T cell receptor alpha chain. In certain embodiments, for example, the at least one T cell receptor component can comprise at least one T cell receptor beta chain. In certain embodiments, for example, the at least one T cell receptor component can comprise at least one pair of T cell receptor alpha and beta chains. In certain embodiments, for example, the identifying can comprise: sequencing the at least one binding T cell at a single cell level. In certain embodiments, for example, the identifying can comprise: sequencing the at least one functional T cell at a single cell level. In certain embodiments, for example, the at least one T cell receptor component can comprise at least one CDR3 sequence. In certain embodiments, for example, the at least one CDR3 sequence can comprise an amino acid sequence (for example a linear sequence) consisting of between 16 and 106 amino acids. In some embodiments, the at least one CDR3 sequence can comprise an amino acid sequence (for example a linear sequence) consisting of between 6 and 35 amino acids. In yet further embodiments, the at least one CDR3 sequence can comprise an amino acid sequence (for example a linear sequence) consisting of between 6 and 12 amino acids. In certain embodiments, for example, the at least one CDR3 sequence can be exclusive of stem regions. In certain embodiments, for example, the at least one CDR3 sequence can comprise CDR3 beta chain portions.

Sequencing of the T cell receptor can be performed using methods known in the art, such as those disclosed in the INCORPORATED REFERENCES. For example, by way of example and without limitation, sequencing can be performed by restriction enzyme digestion of query DNA, followed by gel electrophoresis and Southern blotting using probes for the known T cell receptor genes; next generation sequencing (NGS) (e.g., Illumina sequencing platforms, IonTorrent, or Pacific Biosciences); or PCR-based assays. There are several approaches for extracting CDR data from sequencing reads and determining the clonotype. For example, one exemplary strategy for characterizing CDR3 sequences is T cell receptor profiling, which amplifies cDNA or genomic DNA from the T cell receptor beta-chain CDR3 (β-CDR3) locus using predesigned PCR primers, followed by deep sequencing. Another exemplary approach, involves T cell receptor profiling based on RNA sequencing (RNA-seq), and provides data from all transcribed genes present in the sample, as well as enabling simultaneous analysis of TCRα, TCRβ, TCRγ and TCRδ. However, it is understood that the methods described above are merely exemplary, and that any sequencing method known in the art can be used to determine the T cell receptor sequence.

In certain embodiments, for example, the identifying at least one T cell receptor component of antigen-binding and antigen-activated T cells can comprise comparing T cell receptors from a first sample containing antigen-binding T cells (some of which can or can not be antigen-activated) with a second sample containing antigen-activated T cells (some of which can or can not be antigen-binding). In certain embodiments, for example, the comparing can comprise matching any of the foregoing T cell receptor components between a T cell from the first sample and a T cell from the second sample.

In certain embodiments, for example, the predetermined type of antigen can be a peptide. In certain embodiments, for example, the peptide can consist of at least 8 amino acids. In some embodiments, the peptide consists of 9 amino acids. In some embodiments, the peptide consists of 10 amino acids. In some embodiments, the peptide consists of 11 amino acids. In some embodiments, the peptide consists of 12 amino acids. In some embodiments, the peptide consists of 13 amino acids. In some embodiments, the peptide consists of 14 amino acids. In some embodiments, the peptide consists of 15 amino acids. However, it is understood that the peptide can also consist of more than 15 amino acids, and that the above peptide lengths are merely exemplary.

In certain embodiments, for example, the predetermined type of antigen can be a peptide that is between 8 and 20 amino acids. For example, in some embodiments, the peptide can consist of between 8 and 15 amino acids. In some embodiments, the peptide can consist of between 8 and 12 amino acids. In certain embodiments, for example, the peptide can consist of at least 12 amino acids, for example 13 amino acids, 14 amino acids, 15 amino acids, 16 amino acids, 17 amino acids, 18 amino acids, 19 amino acids, 20 amino acids, 21 amino acids, 22 amino acids, 23 amino acids, 24 amino acids, 25 amino acids, 26 amino acids, 27 amino acids, 28 amino acids, 29 amino acids, 30 amino acids, 31 amino acids, 32 amino acids, 33 amino acids, 34 amino acids, 35 amino acids, 36 amino acids, 37 amino acids, 38 amino acids, 39 amino acids, or 40 amino acids.

In certain embodiments, for example, the predetermined type of antigen can be a peptide that is between 12 and 40 amino acids. In specific embodiments, the peptide can consist of between 12 and 30 amino acids. In some embodiments, the peptide can consist of between 12 and 20 amino acids.

Major histocompatibility complex (MHC) class I and class II proteins share the task of presenting peptides on the cell surface for recognition by T cells. Immunogenic peptide-MHC class I (pMHCI) complexes are presented on nucleated cells and are recognized by cytotoxic CD8+ T cells. The presentation of pMHCII by antigen-presenting cells [e.g., dendritic cells (DCs), macrophages, or B cells], on the other hand, can activate CD4+ T cells, leading to the coordination and regulation of effector cells. In all cases, it is a clonotypic T cell receptor that interacts with a given pMHC complex, potentially leading to sustained cell:cell contact formation and T cell activation.

Accordingly, in certain embodiments, for example, the predetermined type of antigen (for example a peptide) can have a binding affinity for an MHC protein (for example an MHC Class I protein or an MHC Class II protein). In certain embodiments, for example, the predetermined type of antigen can have a binding affinity for an MHC Class I protein of less than 1000 μM. In some embodiments, the predetermined type of antigen can have a binding affinity for an MHC Class I protein of less than 100 μM. In some embodiments, the predetermined type of antigen can have a binding affinity for an MHC Class I protein of less than 50 μM. In some embodiments, the predetermined type of antigen can have a binding affinity for an MHC Class I protein of less than 10 μM. In some embodiments, the predetermined type of antigen can have a binding affinity for an MHC Class I protein of less than 1 μM. In some embodiments, the predetermined type of antigen can have a binding affinity for an MHC Class I protein of less than 0.1 μM.

In certain embodiments, for example, the predetermined type of antigen can be a neoantigen (for example an antigen that has at least one alteration that makes it distinct from the corresponding wild-type, parental antigen). In certain embodiments, for example, the neoantigen can comprise an alteration to a parental antigen via mutation in a tumor cell. In certain embodiments, for example, the mutation can comprise a frameshift or nonframeshift indel. In certain embodiments, for example, the mutation can comprise a missense substitution. In certain embodiments, for example, the mutation can comprise a nonsense substitution. In certain embodiments, for example, the mutation can comprise a splice site alteration. In certain embodiments, for example, the mutation can comprise a genomic rearrangement. In certain embodiments, for example, the mutation can comprise a gene fusion. In certain embodiments, for example, the mutation can comprise a genomic rearrangement and/or expression alteration giving rise to a neoORF. In certain embodiments, for example, the genomic rearrangement can comprise one or more insertions. In certain embodiments, for example, the genomic rearrangement can comprise one or more deletions. In certain embodiments, for example, the mutation can comprise a splice variant. In certain embodiments, for example, the neoantigen can comprise an alteration to a parental antigen via post-translational modification specific to a tumor cell. In certain embodiments, for example, the post-translational modification can comprise aberrant phosphorylation. In certain embodiments, for example, the post-translational modification can comprise a proteasome-generated spliced antigen. In certain embodiments, for example, the neoantigen can be derived from a tumor. In certain embodiments, for example, the tumor can be a solid tumor. In certain embodiments, for example, the neoantigen can be presented on a tumor. In certain embodiments, for example, the neoantigen can be a tumor neoantigen. In certain embodiments, for example, the tumor neoantigen can be present in a subject's tumor cell or tissue but not in the subject's corresponding normal cell or tissue. In certain embodiments, for example, the tumor neoantigen can be overexpressed in a subject's tumor cell or tissue relative to expression in the subject's corresponding normal cell or tissue. In certain embodiments, for example, the neoantigen can be a personalized neoantigen. In certain embodiments, for example, the neoantigen can be a shared tumor neoantigen. In certain embodiments, for example, the shared tumor neoantigen can be a tumor associated peptide neoantigen. In certain embodiments, for example, the neoantigen can be characteristic of a particular type of tumor. In certain embodiments, for example, the neoantigen can be a tumor associated peptide neoantigen. In certain embodiments, for example, the neoantigen can be selected from one or more neoantigens identified by a model. In certain embodiments, for example, the one or more neoantigens can be personalized neoantigens. In certain embodiments, for example, the one or more neoantigens can be present in a list of shared neoantigens. In certain embodiments, for example, the neoantigen can be selected from one or more neoantigens identified by an artificial intelligence model. In certain embodiments, for example, the model can be calibrated using machine learning. In certain embodiments, for example, the artificial intelligence model can comprise a neural network. In certain embodiments, for example, the neoantigen can be selected from a set of presentation likelihoods. In certain embodiments, for example, the neoantigen can be determined using one or more of the machine learning methods, software, and/or systems disclosed in the INCORPORATED REFERENCES.

In certain embodiments, for example, the neoantigen can be a tumor neoantigen. In certain embodiments, for example, the tumor neoantigen can be present in a subject's tumor cell or tissue but not in the subject's corresponding normal cell or tissue. In certain embodiments, for example, the tumor neoantigen can be overexpressed in a subject's tumor cell or tissue relative to expression in the subject's corresponding normal cell or tissue. In certain embodiments, for example, the tumor neoantigen can be determined using one or more of the machine learning methods, software, and/or systems disclosed in the INCORPORATED REFERENCES.

In certain embodiments, for example, the neoantigen can be associated with a type of cancer. In certain embodiments, for example, the cancer can be selected from the group consisting of lung cancer, bladder cancer, stomach cancer, rectal cancer, endometrial cancer, thyroid cancer, renal papillary cell, melanoma, breast cancer, ovarian cancer, prostate cancer, kidney cancer, gastric cancer, colon cancer, testicular cancer, head and neck cancer, pancreatic cancer, brain cancer (e.g., lower grade glioma, glioblastoma), B-cell lymphoma, acute myelogenous leukemia, chronic myelogenous leukemia, chronic lymphocytic leukemia, and T cell lymphocytic leukemia, non-small cell lung cancer (e.g., squamous-cell carcinoma (SCC)), and small cell lung cancer, and combinations of two or more of the foregoing cancers. In certain embodiments, for example, the cancer can be selected from subgroups of the foregoing group. In certain embodiments, for example, the cancer can be an epithelial cancer. In certain embodiments, for example, the cancer can be a blood cancer.

Accordingly, also provided herein in a method for screening a candidate neoantigen for immunogenicity, that includes isolating, from a population of PBMCs, at least one T cell that binds to the candidate neoantigen; forming a plurality of cognate T cells, comprising: expanding the isolated at least one T cell; and activating at least a first functional T cell, comprising: contacting T cells derived from the plurality of cognate T cells with at least one of a plurality of activation agents that is immunogenic for the candidate neoantigen.

The present disclosure also provides for a method for screening a candidate antigen for an antigen-specific vaccine. In some embodiments, the method comprises isolating, from a population of PBMCs, at least one T cell that binds to the candidate antigen; forming a plurality of cognate T cells, comprising: expanding the isolated at least one T cell; and activating at least a first functional T cell, comprising: contacting T cells derived from the plurality of cognate T cells with at least one of a plurality of activation agents that is immunogenic for the candidate antigen.

In certain embodiments, the predetermined type of antigen can be an antigen selected from a publically available database that contains curated T-cell receptor (TCR) sequences with known antigen specificities, such as the VDJdb database (vdjdb.cdr3.net). In some embodiments, the predetermined type of antigen is a predicted antigen. In other embodiments, the predetermined type of antigen is an experimentally verified antigen.

In certain embodiments, for example, source T cells for the systems and methods can be provided. In certain embodiments, for example, the source T cells can be derived from PBMCs. In certain embodiments, for example, the source T cells can be derived from bone marrow. In certain embodiments, for example, the source T cells can be derived from a thymus. In certain embodiments, for example, the source T cells can be derived from a tissue biopsy. In certain embodiments, for example, the source T cells can be derived from a tumor. In certain embodiments, for example, the source T cells can be derived from a lymph node tissue. In certain embodiments, for example, the source T cells can be derived from a gut associated lymphoid tissue. In certain embodiments, for example, the source T cells can be derived from a mucosa associated lymphoid tissue. In certain embodiments, for example, the source T cells can be derived from a spleen tissue. In certain embodiments, for example, the source T cells can be derived from a lymphoid tissue. In certain embodiments, for example, the source T cells can be derived from a tumor (for example one of the tumors disclosed herein). In certain embodiments, for example, the source T cells can be obtained from a T cell line. In certain embodiments, for example, the source T cells can be obtained from an autologous source. In certain embodiments, for example, the source T cells can be obtained from an allogeneic source. In certain embodiments, for example, the source T cells can be obtained from a single individual. In certain embodiments, for example, the single individual can be healthy (for example free of a preselected one or more diseases). In certain embodiments, for example, the single individual can suffer from one or more preselected diseases (for example a preselected cancer). In certain embodiments, for example, the source T cells can be obtained from a population of individuals. In certain embodiments, for example, the population of individuals can be healthy (for example free of a preselected one or more diseases). In certain embodiments, for example, the population of individuals can suffer from one or more preselected diseases (for example a preselected cancer).

In certain embodiments, for example, the source T cells can be derived from cells obtained by leukapheresis of circulating blood of an individual. In certain embodiments, for example, the source T cells can be derived from cells obtained by apheresis of circulating blood of an individual. In certain embodiments, for example, obtained cells can comprise lymphocytes. In certain embodiments, for example, the obtained lymphocytes can comprise T cells and optionally one or more of monocytes, granulocytes, B cells, other nucleated while blood cells, red blood cells, and platelets.

In certain embodiments, for example, the obtained cells can be washed to remove plasma and to place the cells in an appropriate buffer or media for subsequent processing to obtain the source T cells. In certain embodiments, for example, the cells can be washed with phosphate buffered saline (PBS). In certain embodiments, for example, the wash solution can be exclusive of calcium cations, magnesium cations, or all divalent cations. In certain embodiments, for example, the washing can use a semi-automated flow-through centrifuge. In certain embodiments, for example, the washing can be following by resuspending in a liquid. In certain embodiments, for example, the liquid can comprise a biocompatible buffer. In certain embodiments, for example, the biocompatible buffer can comprise a calcium cation-free and magnesium cation-free, PBS. In certain embodiments, for example, undesirable components of cells obtained by apheresis can be removed and the cells directly resuspended in culture media.

In certain embodiments, for example, the at least one T cell receptor component can be present in less than 1 T cell in 1,000,000 of the source T cells. For example, in some embodiments, the at least one T cell receptor component can be present in less than 1 T cell in 2,000,000 of the source T cells. In some embodiments, the at least one T cell receptor component can be present in less than 1 T cell in 5,000,000 of the source T cells. In some embodiments, the at least one T cell receptor component can be present in less than 1 T cell in 10,000,000 of the source T cells.

In certain embodiments, for example, the methods disclosed herein can be exclusive of T cell priming. In certain embodiments, for example, the methods disclosed herein can eliminate the need to isolate antigen presenting cells from blood samples for the purpose of priming T cells. In certain embodiments, for example, elimination of the need to isolate antigen presenting cells from blood samples for the purpose of priming T cells can reduce to total volume of blood required by at least 25% (for example at least 50% or between 30% and 70%) compared to methods that utilize T cell priming.

Various methods for isolating T cells are known in the art, and it is understood that any of the isolation methods can be used in combination with the present invention. In certain embodiments, for example, source T cells can be isolated from peripheral blood lymphocytes by lysing red blood cells and by centrifugation through a PERCOLL™ gradient. In certain embodiments, for example, source T cells can be isolated from peripheral blood lymphocytes by Ficoll-Paque separation. In certain embodiments, for example, source T cells can be isolated from peripheral blood lymphocytes using a microfluidic device. In certain embodiments, for example, positive or negative selection can be used to obtain the mixture of T cells from the source T cells. In certain embodiments, for example, the mixture of T cells can comprise (or be enriched for) CD28⁺ T cells. In certain embodiments, for example, the mixture of T cells can comprise (or be enriched for) naïve CD8⁺ T cells. In certain embodiments, for example, the mixture of T cells can comprise (or be enriched for) naïve T cells. In certain embodiments, for example, the mixture of T cells can comprise (or be enriched for) memory T cells. In certain embodiments, for example, the mixture of T cells can comprise (or be enriched for) CD8⁺ T cells. In certain embodiments, for example, the mixture of T cells can comprise (or be enriched for) CD4⁺ T cells. In certain embodiments, for example, the mixture of T cells can comprise (or be enriched for) CD4⁺ CD8⁺ T cells. In certain embodiments, for example, the mixture of T cells can comprise (or be enriched for) CD4⁻ CD8⁺ T cells. In certain embodiments, for example, the mixture of T cells can comprise (or be enriched for) CD4⁺ CD8⁻ T cells. In certain embodiments, for example, the mixture of T cells can comprise (or be enriched for) CD45RA⁺. In certain embodiments, for example, the mixture of T cells can comprise (or be enriched for) CD45RO⁺ T cells. In certain embodiments, for example, the mixture of T cells can comprise (or be enriched for) CD3⁺/CD28⁺ T cells.

As described above, positive selection, negative selection, or a combination of both can be used to obtain the desired population of T cells from the source T cells. In certain embodiments, for example, T cells can be positively selected by conjugating anti-marker agents (for example antibodies) to magnetic beads and performing magnetic separation. In other embodiments, for example, a T cell subpopulation can be negatively selected by conjugating antibodies to surface markers unique to the undesired cells of the T cell subpopulation. In certain embodiments, for example, the negative selection can comprise cell sorting. In certain embodiments, for example, the negative selection can comprise selection via negative magnetic immunoadherence. In certain embodiments, for example, the negative selection can comprise selection via flow cytometry using a cocktail of monoclonal antibodies directed to cell surface markers present on the cells negatively selected. In certain embodiments, for example, a mixture of T cells enriched in CD4⁺ T cells can be obtained by exposing source T cells to monoclonal antibodies (for example biotinylated monoclonal antibodies which can be coupled to anti-biotin magnetic beads) for one or more (for example all) of CD14, CD20, CD11b, CD16, HLA-DR, and CD8, followed by enrichment (for example including magnetic separation) and characterization by flow cytometry. In certain embodiments, for example, a mixture of T cells enriched in CD8⁺ T cells can be obtained by exposing source T cells to monoclonal antibodies (for example biotinylated monoclonal antibodies which can be coupled to anti-biotin magnetic beads) for one or more (for example all) of CD45RO, CD14, CD15, CD16, CD19, CD25, CD34, CD36, CD57, CD123, anti-HLA-DR, CD235a (Glycophorin A), CD244, and CD4 followed by enrichment (for example including magnetic separation) and characterization by flow cytometry. It is understood that the examples described above for positive selection, negative selection, or a combination of both, are non-limiting and that any marker specific to the desired cell population can be used for positive selection or any marker specific to the undesired cell population can be used for negative selection.

Accordingly, also provided herein is a method for negative selection of T cell receptor clonotypes. In some embodiments, the method includes analyzing a mixture of T cells to identify first antigen-binding T cells and first antigen-activated T cells for a predetermined first type of antigen and second antigen-activated T cells for a predetermined second type of antigen; and identifying at least a portion of at least one T cell receptor sequence shared by at least one of the first antigen-binding T cells and at least one of the first antigen-activated T cells; and not shared with any of the second antigen-activated T cells. In other embodiments, the method for negative selection of T cell receptor clonotypes, includes analyzing a mixture of T cells to identify first antigen-activated T cells and first antigen-binding T cells for a predetermined first type of antigen and second antigen-binding T cells for a predetermined second type of antigen; and identifying at least a portion of at least one T cell receptor sequence shared by at least one of the first antigen-binding T cells and at least one of the first antigen-activated T cells; and not shared with any of the second antigen-binding T cells.

In certain embodiments, for example, the mixture of T cells can be prepared by freezing washed (for example washed as described herein) source T cells in a freezing solution. In certain embodiments, for example, the freezing solution can comprise PBS. In certain embodiments, for example, the PBS can contain dimethyl sulfoxide (DMSO) (for example 5-40% DMSO, such as 20% DMSO). In certain embodiments, for example, the PBS can contain and human serum albumin (HSA) (for example 1-30% HSA such as 8% HSA). In certain embodiments, for example, the freezing solution can contain other suitable cell freezing components. In certain embodiments, for example, the freezing solution can be a non-diluted freezing solution. In certain embodiments, for example, the freezing solution can be diluted. In certain embodiments, for example, the freezing solution can comprise PBS containing 20% DMSO and 8% human serum albumin (HSA) (or other suitable cell freezing components) diluted 1:1 with media. In certain embodiments, for example, the mixture of T cells can be frozen to −80° C. and stored in the vapor phase of a liquid nitrogen storage tank.

In certain embodiments, for example, the at least one T cell receptor component can be present in less than 1 T cell in 1,000 of the T cells in the T cell mixture. For example, in some embodiments, the at least one T cell receptor component can be present in less than 1 T cell in 10,000 of the T cells in the T cell mixture. In some embodiments, the at least one T cell receptor component can be present in less than 1 T cell in 100,000 of the T cells in the T cell mixture. In some embodiments, the at least one T cell receptor component can be present in less than 1 T cell in 1,000,000 of the T cells in the T cell mixture. In some embodiments, the at least one T cell receptor component can be present in less than 1 T cell in 10,000,000 of the T cells in the T cell mixture.

In certain embodiments, for example, the at least one T cell receptor component can be present in at least 0.0005% of the T cells in the T cell mixture. For example, in some embodiments, the at least one T cell receptor component can be present in at least 0.005% of the T cells in the T cell mixture. In some embodiments, the at least one T cell receptor component can be present in at least 0.05% of the T cells in the T cell mixture. In some embodiments, the at least one T cell receptor component can be present in at least 0.5% of the T cells in the T cell mixture. In some embodiments, the at least one T cell receptor component can be present in at least 5% of the T cells in the T cell mixture. In some embodiments, the at least one T cell receptor component can be present in at least 10% of the T cells in the T cell mixture. In some embodiments, the at least one T cell receptor component can be present in at least one T cell receptor component can be present in at least 15% of the T cells in the T cell mixture.

In certain embodiments, for example, the at least one T cell receptor component can be present in less than 5% of the T cells in the T cell mixture. In some embodiments, the at least one T cell receptor component can be present in less than 0.5% of the T cells in the T cell mixture. In some embodiments, the at least one T cell receptor component can be present in less than 0.005% of the T cells in the T cell mixture. In some embodiments, the at least one T cell receptor component can be present in at least one T cell receptor component can be present in less than 0.0005% of the T cells in the T cell mixture.

Certain embodiments can comprise, for example, enriching a subpopulation of T cells from a population of T cells (for example using one or more of the methods, assays, or systems disclosed in the INCORPORATED REFERENCES). In certain embodiments, for example, the enriching can comprise enriching a subpopulation of T cells that exhibiting antigen-binding or antigen-activation for a predetermined type of antigen. In certain embodiments, for example, the enriching can comprise contacting the population of T cells with a binding agent, followed by separating a member of the subpopulation bound to the binding agent from the population of T cells.

In certain embodiments, for example, the binding agent can comprise at least a portion of the predetermined type of antigen (or a portion of the antigen). In certain embodiments, for example, the binding agent can comprise at least a portion of the predetermined type of antigen bound to at least a portion of an MHC protein. In certain embodiments, for example, the MHC protein can be an MHC Class I protein. In certain embodiments, for example, the MHC protein can be an MHC Class II protein. In certain embodiments, for example, the binding agent can comprise at least a portion of the predetermined type of antigen bound to an aptamer. In certain embodiments, for example, the binding agent can comprise at least a portion of the predetermined type of antigen bound to an affimer. In certain embodiments, for example, the binding agent can comprise at least a portion of the predetermined type of antigen bound to an antibody. In certain embodiments, for example, the binding agent can comprise a multimer (for example a tetramer comprising at least a portion of the predetermined type of antigen bound to at least a portion of an MHC protein). In certain embodiments, for example, the binding agent can be linked to a magnetic bead to facilitate magnetic separation or a fluorophore to facility isolation via fluorescence flow cytometry.

In certain embodiments, for example, the member can bind with the binding agent with a dissociation constant of between 0.01 μM and 1000 μM. For example, in some embodiments, the member can bind with the binding agent with a dissociation constant of between 0.1 μM and 100 μM. In some embodiments, the member can bind with the binding agent with a dissociation constant of between 0.5 μM and 50 μM. In some embodiments, the member can bind with the binding agent with a dissociation constant of between 1 μM and 50 μM. In some embodiments, the member can bind with the binding agent with a dissociation constant of between 1 μM and 25 μM. In some embodiments, the member can bind with the binding agent with a dissociation constant of between 25 μM and 75 μM. In some embodiments, the member can bind with the binding agent with a dissociation constant between 10 μM and 50 μM.

In further embodiments, for example, the member can bind with the binding agent with a dissociation constant of less than 1000 μM. In some embodiments, the member can bind with the binding agent with a dissociation constant of less than 100 μM. In some embodiments, the member can bind with the binding agent with a dissociation constant of less than 75 μM. In some embodiments, the member can bind with the binding agent with a dissociation constant of less than 50 μM. In some embodiments, the member can bind with the binding agent with a dissociation constant of less than 40 μM. In some embodiments, the member can bind with the binding agent with a dissociation constant of less than 30 μM. In some embodiments, the member can bind with the binding agent with a dissociation constant of less than 20 μM. In some embodiments, the member can bind with the binding agent with a dissociation constant less than 10 μM.

In certain embodiments, for example, the member can bind with the binding agent with a half-life of between 0.1 seconds and 100 seconds. For example, in some embodiments, the member can bind with the binding agent with a half-life of between 1 second and 50 seconds. In some embodiments, the member can bind with the binding agent with a half-life of between 1 second and 25 seconds. In some embodiments, the member can bind with the binding agent with a half-life of between 1 second and 10 seconds. In some embodiments, the member can bind with the binding agent with a half-life of between 2 seconds and 10 seconds. In some embodiments, the member can bind with the binding agent with a half-life of between 2 seconds and 7 seconds. In some embodiments, the member can bind with the binding agent with a half-life of between 2 seconds and 5 seconds.

In certain embodiments, for example, the member can bind with the binding agent with a half-life of at least 0.1 seconds. For example, in some embodiments, the member can bind with the binding agent with a half-life of at least 0.5 seconds. In some embodiments, the member can bind with the binding agent with a half-life of at least 1 second. In some embodiments, the member can bind with the binding agent with a half-life of at least 2 seconds. In some embodiments, the member can bind with the binding agent with a half-life of at least 5 seconds. In some embodiments, the member can bind with the binding agent with a half-life of at least 10 seconds.

In certain embodiments, for example, the member can bind with the binding agent with dissociation constant of less than 50 μM and a half-life of between 2 seconds and 10 seconds.

Certain embodiments can comprise, for example, expanding one or more T cells (for example using one or more of the methods, assays, or systems disclosed in the INCORPORATED REFERENCES). In certain embodiments, for example, the expanding can comprise expanding a plurality of T cells that have been positively selected as exhibiting antigen-binding or antigen-activation for a predetermined type of antigen). In certain embodiments, for example, the expanding can comprise polyclonal expansion.

Certain embodiments can comprise, for example, progressively enriching a population of antigen-binding T cells for a predetermined type of antigen by a serially enriching followed by expanding a starting mixture of T cells a number of times, for example 2 times (i.e., enrich expand enrich or enrich expand enrich expand). In some embodiments, the starting mixture of T cells can be expanded at least 3 times. In some embodiments, the starting mixture of T cells can be expanded at least 4 times. In some embodiments, the starting mixture of T cells can be expanded at least 5 times. In some embodiments, the starting mixture of T cells can be expanded greater than 5 times. In certain embodiments, for example, a portion of the progressively enriched population (i.e., the population of T cells that results after the serial enrichment) can be further selected for antigen-activation (for example by exposing the progressively enriched population of antigen-binding T cells to cells presenting the predetermined type of antigen and further enriching based on the presence of one or more activation markers.

In certain embodiments, for example, the antigen-activated T cells can be formed by contacting a T cell with an activation agent, and the resulting antigen-activated T cell detected by detecting by expression of one or more activation markers and/or secreted molecules. In certain embodiments, for example, antigen-activated T cells can be formed by contacting T cells with activation agents, and the resulting antigen-activated T cell detected by detecting expression of one or more activation markers as disclosed herein or in one of the INCORPORATED REFERENCES.

In certain embodiments, for example, the one or more activation markers can comprise a cell surface marker. In certain embodiments, for example, the one or more activation markers can comprise a signaling molecule (for example a molecule upregulated in response to T cell activation). In certain embodiments, for example, the one or more activation markers and/or secreted molecules can comprise CD137 (also known as 4-1BB, or Tnsfr9), interferon gamma (IFN-γ), tumor necrosis factor alpha (TNFα), interleukin-2 (IL-2), CD69, upregulation of an MHC Class I protein, upregulation of a MHC Class II protein, Ki67, perforin, granzyme, CD122, CD27, CD28, CD95, CD134, killer-cell lectin like receptor G1 (KLRG1), CD38, CD154, or a combination of two or more of the foregoing. In specific embodiments, the activation maker can be CD137. In other embodiments, the activation maker can be IFN-γ. In some embodiments, the activation marker can be TNFα. In some embodiments, the activation marker can be IL-2. In some embodiments, the activation marker can be CD69. In some embodiments, the activation marker can be upregulation of an MHC Class I protein. In some embodiments, the activation marker can be upregulation of an MHC Class II protein. In some embodiments, the activation marker can be Ki67. In some embodiments, the activation marker can be CD137 and IFN-γ. In some embodiments, the activation marker can be CD137, IFN-γ, and IL-2. It is understood that the exemplary activation markers described above are non-limiting, and that any T cell activation marker known in the art can be used with the disclosures provided herein.

The present disclosure also provides for method for identifying a T cell activation marker. In some embodiments, the method includes contacting a first plurality of T cells with a plurality of P-presenting cells, the first plurality of T cells comprising a plurality of P-binding T cells, which P is a predetermined type of antigen; measuring a plurality of expression rate profiles for at least a portion of the contacted plurality of P-binding T cells; partitioning, into a plurality of T cell clusters, the at least a portion of the contacted plurality of P-binding T cells; measuring a functional response to P in at least two T cells present in the at least a portion of the contacted plurality of P-binding T cells; mapping the expression rate profiles to the plurality of T cell clusters to identify one of the plurality of T cell clusters comprising the at least two T cells; and identifying an activation marker that is expressed by the at least two T cells.

In certain embodiments, for example, two or more of the activation markers and/or secreted molecules can be detected on separate portions of T cells and individual T cells between the two portions that share the same at least one T cell component identified. In certain embodiments, for example, two or more of the activation markers and/or secreted molecules can be detected together in a single portion of T cells.

In certain embodiments, for example, the activation agents can comprise the predetermined type of antigen. In certain embodiments, for example, the activation agents can further comprise a costimulatory ligand. In certain embodiments, for example, the costimulatory ligand can be one or more of the ligands selected from the group consisting of: an antibody or antigen-binding fragment thereof that specifically binds to CD28, CD80 (B7-1), CD86 (B7-2), B7-H3, 4-1BBL, 4-1BB, CD27, CD30, CD134 (OX-40L), B7h (B7RP-1), CD40, LIGHT, an antibody or antigen-binding fragment thereof that specifically binds to HVEM, an antibody or antigen-binding fragment thereof that specifically binds to CD40L, an antibody or antigen binding fragment thereof that specifically binds to OX40, and an antibody or antigen-binding fragment thereof that specifically binds to 4-1BB. In certain embodiments, for example, the costimulatory ligand can be selected from the group consisting of a monoclonal antibody, a F(ab′)2, a Fab, scFv, and a single chain antibody. In certain embodiments, for example, the costimulatory ligand can be a humanized monoclonal antibody or fragment. In certain embodiments, for example, the costimulatory ligand can be a humanized murine monoclonal antibody or fully human antibody against CD28. In certain embodiments, for example, the costimulatory ligand can be a humanized monoclonal antibody or antigen-binding fragment thereof. In certain embodiments, for example, the predetermined type of antigen (for example a predetermined type of antigen complexed with an MHC protein) and costimulatory ligand are covalently bound to the surface of a paramagnetic particle.

In certain embodiments, for example, the activation agents can comprise the predetermined type of antigen on the surface of a cell. In certain embodiments, for example, the activation agents can comprise one or more dendritic cells. In certain embodiments, for example, the activation agents can comprise one or more antigen presenting cells (for example one or more professional antigen presenting cells). In certain embodiments, for example, the activation agents can comprise one or more artificial antigen presenting cells. In certain embodiments, for example, the activation agents can comprise one or more macrophages. In certain embodiments, for example, the activation agents can comprise one or more B cells.

In certain embodiments, for example, the activation agents can comprise one or more cancer cells. For example, in some embodiments, the cancer cell is from a solid tumor. In some embodiments, the cancer cell is from a hematological malignancy. In yet further embodiments, the cancer cell is a circulating tumor cell. In certain embodiments, for example, the cancer can be selected from the group consisting of lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T cell lymphocytic leukemia, non-small cell lung cancer, and small cell lung cancer, and combinations of two or more of the foregoing cancers. In certain embodiments, for example, the cancer can be selected from subgroups of the foregoing group.

In certain embodiments, for example, the activation agents can comprise one or more cells for a cancerous tumor. In certain embodiments, for example, the tumor can be selected from the group consisting of tumors for lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T cell lymphocytic leukemia, non-small cell lung cancer, and small cell lung cancer, and combinations of two or more of the foregoing cancers. In certain embodiments, for example, the tumor can be selected from subgroups of the foregoing group.

In certain embodiments, any of the foregoing activation agents can be formed by antigen-loading with a quantity of the predetermined type of antigen. In certain embodiments, for example, the antigen-loading can be configured to present a predetermined concentrations of the predetermined type of antigen on the activation agents (for example each of the activation agents can have a similar concentration of the predetermined type of antigen). In certain embodiments, for example, the activation agent can be formed exclusive of antigen-loading.

In certain embodiments, for example, the activation agents can present the predetermined type of antigen at a physiologically relevant concentration. In certain embodiments, for example, the activation agents can present the predetermined type of antigen at a concentration determined by pulsing the plurality of cells with a solution containing the predetermined type of antigen for a predetermined period of time, the solution containing the predetermined type of antigen at a concentration of between 0.000001 μM and 100 μM. For example, in some embodiments, the solution containing the predetermined type of antigen is at a concentration of between 0.000001 μM and 0.00001 μM. In some embodiments, the solution containing the predetermined type of antigen is at a concentration of between 0.00001 μM and 0.0001 μM. In some embodiments, the solution containing the predetermined type of antigen is at a concentration of between 0.0001 μM and 0.001 μM. In some embodiments, the solution containing the predetermined type of antigen is at a concentration of between 0.001 and 0.01 μM. In some embodiments, the solution containing the predetermined type of antigen is at a concentration of between 0.01 and 0.1 μM. In some embodiments, the solution containing the predetermined type of antigen is at a concentration of between 0.0001 μM and 100 μM. In some embodiments, the solution containing the predetermined type of antigen is at a concentration of between 0.001 μM and 100 μM. In some embodiments, the solution containing the predetermined type of antigen is at a concentration of between 0.01 μM and 10 μM. In some embodiments, the solution containing the predetermined type of antigen is at a concentration of between 0.1 μM and 10 μM. In some embodiments, the solution containing the predetermined type of antigen is at a concentration of between 1 μM and 100 μM. In some embodiments, the solution containing the predetermined type of antigen is at a concentration of between 1 μM and 50 μM. In some embodiments, the solution containing the predetermined type of antigen is at a concentration of between 1 μM and 25 μM. In some embodiments, the solution containing the predetermined type of antigen is at a concentration of between 5 μM and 25 μM. In some embodiments, the solution containing the predetermined type of antigen is at a concentration of between 10 μM and 100 μM. In some embodiments, the solution containing the predetermined type of antigen is at a concentration of between 10 μM and 30 μM.

In certain embodiments, for example, the solution can contain the predetermined type of antigen at a concentration of less than 100 μM. For example, in some embodiments, the solution can contain the predetermined type of antigen at a concentration of less than 75 μM. In some embodiments, the solution can contain the predetermined type of antigen at less than 50 μM. In some embodiments, the solution can contain the predetermined type of antigen at less than 25 μM. In some embodiments, the solution can contain the predetermined type of antigen at less than 10 μM. In some embodiments, the solution can contain the predetermined type of antigen at less than 1 μM.

In any of the foregoing embodiments, for example, the predetermined period of time can be between 1 hr and 36 hrs. For example, in some embodiments, the predetermined period of time can be between 6 hours and 24 hours. In some embodiments, the predetermined period of time can be between 6 hours and 12 hours. In some embodiments, the predetermined period of time can be between 12 hours and 24 hours. In some embodiments, the predetermined period of time can be between 9 hours and 18 hours.

In any of the foregoing embodiments, for example, the predetermined period of time can be at least 1 hour. In some embodiments, the predetermined period of time can be at least 4 hours. In some embodiments, the predetermined period of time can be at least 8 hours. In some embodiments, the predetermined period of time can be at least 12 hours. In some embodiments, the predetermined period of time can be at least 18 hours. In some embodiments, the predetermined period of time can be at least 24 hours.

In any of the foregoing embodiments, for example, the predetermined period of time can be less than 168 hours. In some embodiments, the predetermined period of time can be less than 72 hours. In some embodiments, the predetermined period of time can be less than 36 hours. In some embodiments, the predetermined period of time can be less than 24 hours. In some embodiments, the predetermined period of time can be less than 12 hours.

In any of the foregoing embodiments, for example, the predetermined period of time can be repeated one or more times. For example, in some embodiments, the activation agents can present the predetermined type of antigen at any of the concentrations described herein by pulsing the plurality of cells with a solution containing the predetermined type of antigen for any of the predetermined periods of time described herein, and then re-challenged one more time. In some embodiment, the antigen is re-challenged two more times. In further embodiments, the antigen is re-challenged three more times. In yet further embodiments, the antigen is re-challenged four more times. In even further embodiments, the antigen is re-challenged five more times. In other embodiments, the antigen is re-challenged more than five times. In some embodiments, the antigen is re-challenged more than ten times.

In certain embodiments, for example, certain T cells can bind with the activation agents with a dissociation constant of between 0.01 μM and 1000 μM to form activated T cells. For example, in some embodiments, certain T cells can bind with the activation agents with a dissociation constant of between 0.1 μM and 100 μM. In some embodiments, certain T cells can bind with the activation agents with a dissociation constant of between 0.5 μM and 50 μM. In some embodiments, certain T cells can bind with the activation agents with a dissociation constant of between 1 μM and 50 μM. In some embodiments, certain T cells can bind with the activation agents with a dissociation constant of between 1 μM and 25 μM. In some embodiments, certain T cells can bind with the activation agents with a dissociation constant of between 25 μM and 75 μM. In some embodiments, certain T cells can bind with the activation agents with a dissociation constant of between 10 μM and 50 μM.

In certain embodiments, for example, certain T cells can bind with the activation agents with a dissociation constant of less than 1000 μM. In some embodiments, certain T cells can bind with the activation agents with a dissociation constant of less than 100 μM. In some embodiments, certain T cells can bind with the activation agents with a dissociation constant of less than 75 μM. In some embodiments, certain T cells can bind with the activation agents with a dissociation constant of less than 50 μM. In some embodiments, certain T cells can bind with the activation agents with a dissociation constant of less than 40 μM. In some embodiments, certain T cells can bind with the activation agents with a dissociation constant of less than 30 μM. In some embodiments, certain T cells can bind with the activation agents with a dissociation constant of less than 20 μM. In some embodiments, certain T cells can bind with the activation agents with a dissociation constant of less than 10 μM.

In certain embodiments, for example, the certain T cells can bind with the activation agents with a half-life of between 0.1 seconds and 100 seconds to form antigen-activated T cells. For example, in some embodiments, the certain T cells can bind with the activation agents with a half-life of between 1 second and 50 seconds. In some embodiments, the certain T cells can bind with the activation agents with a half-life of between 1 second and 25 seconds. In some embodiments, the certain T cells can bind with the activation agents with a half-life of between 1 second and 10 seconds. In some embodiments, the certain T cells can bind with the activation agents with a half-life of between 2 seconds and 10 seconds. In some embodiments, the certain T cells can bind with the activation agents with a half-life of between 2 seconds and 7 seconds. In some embodiments, the certain T cells can bind with the activation agents with a half-life of between 2 seconds and 5 seconds.

In certain embodiments, for example, the certain T cells can bind with the activation agents with a half-life of at least 0.1 seconds. For example, in some embodiments, the certain T cells can bind with the activation agents with a half-life of at least 0.5 seconds. In some embodiments, the certain T cells can bind with the activation agents with a half-life of at least 1 second. In some embodiments, the certain T cells can bind with the activation agents with a half-life of at least 2 seconds. In some embodiments, the certain T cells can bind with the activation agents with a half-life of at least 5 seconds. In some embodiments, the certain T cells can bind with the activation agents with a half-life of at least 10 seconds.

In certain embodiments, for example, the certain T cells can bind with the activation agents with dissociation constant of less than 50 μM and a half-life of between 2 seconds and 10 seconds to form antigen-activated T cells.

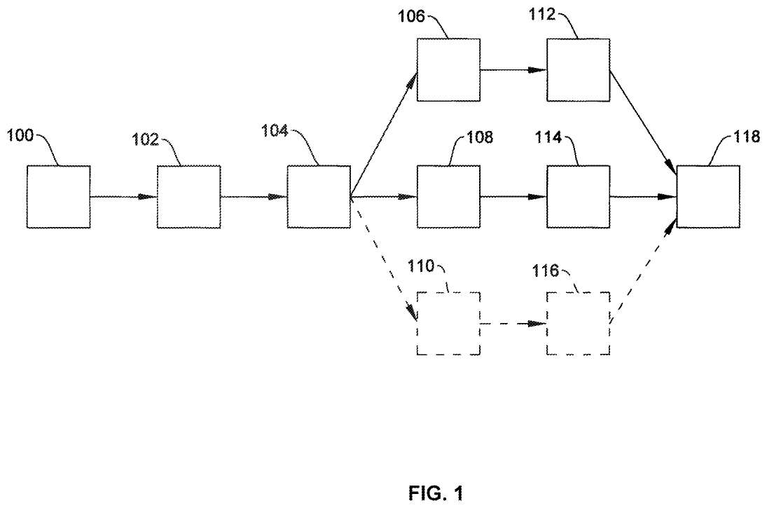

A schematic illustration of a method for identifying T cell receptors having at least portions shared by antigen-binding T cells and antigen-activated T cells for a predetermined type of antigen (for example a neoantigen such as a personalized neoantigen or a shared neoantigen, inclusive of a neoantigen selected by a model whose parameters are adjusted using machine learning) is shown in FIG. 1. Samples are processed 100 to obtain a mixture containing a plurality of different T cells. The mixture can comprise, for example, PBMCs, such as PBMCs obtained by processing leukapheresis samples from healthy donors to remove cells positive for one or more of CD45RO, CD14, CD15, CD16, CD19, CD25, CD34, CD36, CD57, CD123, anti-HLA-DR, CD235a (Glycophorin A), CD244, and CD4. The plurality of different T cells can comprise, for example, naïve CD8⁺ T cells from healthy donors. The mixture is enriched 102 for the naïve CD8⁺ T cells by incubating with P-loaded MHC proteins (where P is the predetermined type of antigen) followed by isolating T cells bound thereto. The P-loaded MHC proteins can be provided, for example, in the form of magnetically-labeled multimers (to facilitate isolation by magnetic separation) or fluorescently-labeled multimers (to facilitate isolation via fluorescence flow cytometry). The isolated T cells are expanded 104, for example by polyclonal expansion, and partitioned into first, second, and optional third T cell populations. The first T cell population is assessed 106 for P-binding T cells by incubating with P-loaded MHC proteins followed by isolating P-binding T cells bound thereto. The second T cell population is assessed 108 for P-activated T cells by exposing to cells presenting the predetermined type of antigen, detecting activation markers or secreted molecules indicative of T cell activation, and isolating activated T cells. The cells presenting the predetermined type of antigen can present, for example, physiologically relevant quantities of the predetermined type of antigen. The cells presenting the predetermined type of antigen can, for example, be professional antigen presenting cells. The cells presenting the predetermined type of antigen can, for example, be tumor cells from a subject. The activation markers can comprise, for example, CD137. The P-activated T cells can be isolated by contacting with magnetically labeled anti-activation marker antibodies followed by magnetic separation. The optional third T cell population is, if present, also assessed 110 for P-activated T cells by exposing to further cells presenting the predetermined type of antigen, detecting further activation markers or further secreted molecules indicative of T cell activation, and isolating further P-activated T cells. T cell receptors from the isolated P-binding T cells and P-activated T cells are sequenced (112, 114, 116) at a single cell level and the resulting sequences compared 118 to identify T cell receptors having at least portions of sequences in common among the P-binding T cells, P-activated T cells, and optionally further P-activated T cells.

FIG. 1 describes certain exemplary embodiments, but other variations fall within scope of the disclosure. In certain embodiments, for example, the PBMCs can be obtained from whole blood samples. In certain embodiments, for example, the plurality of different T cells can comprise CD-4⁺ T cells and the mixture can be enriched for the CD-4⁺ T cells. In certain embodiments, for example, the plurality of different T cells can comprise memory T cells and the mixture can be enriched for the memory T cells. In certain embodiments, for example, the processing can comprise removal of a different panel of biomarkers than shown depending on the desired composition of T cells for enrichment. In certain embodiments, for example, the expanded T cells can be further partitioned into third and fourth T cell populations to test for second and third activation markers and/or secreted molecules. In certain embodiments, for example, the P-activated T cells can be contacting with fluorescently-labeled anti-activation marker antibodies and isolated by passing through a fluorescence flow cytometer.

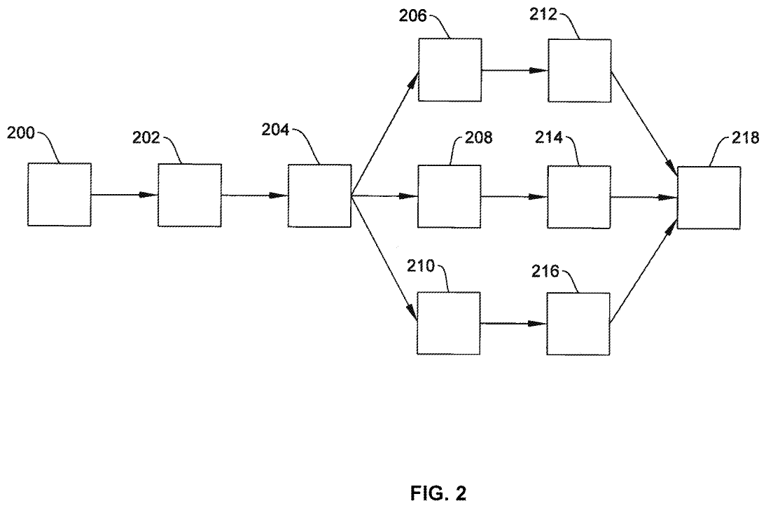

A schematic illustration of a method comprising a negative selection step for identifying T cell receptors having at least portions shared by antigen-binding T cells and antigen-activated T cells for a predetermined type of antigen (for example a neoantigen such as a personalized neoantigen or a shared neoantigen, inclusive of a neoantigen selected by a model whose parameters are adjusted using machine learning) is shown in FIG. 2. Samples are processed 200 to obtain a mixture containing a plurality of different T cells. The mixture can comprise, for example, PBMCs, such as PBMCs obtained by processing leukapheresis samples from healthy donors to remove cells positive for one or more of CD45RO, CD14, CD15, CD16, CD19, CD25, CD34, CD36, CD57, CD123, anti-HLA-DR, CD235a (Glycophorin A), CD244, and CD4. The plurality of different T cells can comprise, for example, naïve CD8⁺ T cells from healthy donors. The mixture is enriched 202 for the naïve CD8⁺ T cells by incubating with P-loaded MHC proteins (which P is the predetermined type of antigen) followed by isolating T cells bound thereto. The P-loaded MHC proteins can be provided, for example, in the form of magnetically-labeled multimers (to facilitate isolation by magnetic separation) or fluorescently-labeled multimers (to facilitate isolation via fluorescence flow cytometry). The isolated T cells are expanded 204, for example by polyclonal expansion, and partitioned into first, second, and third T cell populations. The first T cell population is assessed 206 for P-binding T cells by incubating with P-loaded MHC proteins followed by isolating P-binding T cells bound thereto. The second T cell population is assessed 208 for P-activated T cells by exposing to cells presenting the predetermined type of antigen, detecting activation markers or secreted molecules indicative of T cell activation, and isolating activated T cells. The cells presenting the predetermined type of antigen can present, for example, physiologically relevant quantities of the predetermined type of antigen. The cells presenting the predetermined type of antigen can, for example, be professional antigen presenting cells. The cells presenting the predetermined type of antigen can, for example, be tumor cells from a subject. The activation markers can comprise, for example, CD137. The P-activated T cells can be isolated by contacting with magnetically labeled anti-activation marker antibodies followed by magnetic separation. The third T cell population is assessed 210 for Q-activated T cells by exposing to further cells presenting a different type of antigen Q (which Q is different from P), detecting further activation markers or further secreted molecules indicative of T cell activation, and isolating Q-activated T cells. T cell receptors from the isolated P-binding T cells, P-activated T cells, and Q-activated T cells are sequenced (212, 214, 216) at a single cell level and the resulting sequences compared 218 to identify T cell receptors having at least portions of sequences in common among the P-binding T cells and P-activated T cells but not present among the Q-activated T cells.

FIG. 2 describes certain exemplary embodiments, but other variations fall within scope of the disclosure. In certain embodiments, for example, the PBMCs can be obtained from whole blood samples. In certain embodiments, for example, the plurality of different T cells can comprise CD-4⁺ T cells and the mixture can be enriched for the CD-4⁺ T cells. In certain embodiments, for example, the plurality of different T cells can comprise memory T cells and the mixture can be enriched for the memory T cells. In certain embodiments, for example, the processing can comprise removal of a different panel of biomarkers than shown depending on the desired composition of T cells for enrichment. In certain embodiments, for example, the expanded T cells can be further partitioned into third and fourth T cell populations (or even additional T cell populations) to assess for P-activated and/or Q-activated T cells using additional activation markers and/or secreted molecules. In certain embodiments, for example, the expanded T cells can be negatively selected for T cells activated by further predetermined antigens (i.e., in addition to Q). In certain embodiments, for example, T cells can be negatively selected on the basis of binding to a predetermined antigen rather than activation. In certain embodiments, for example, the P-activated T cells can be contacting with fluorescently-labeled anti-activation marker antibodies and isolated by passing through a fluorescence flow cytometer. In certain embodiments, for example, the third T cell population can be tested in media without Q to detect and isolate T cells showing false-positive results for activation in the absence of antigen.

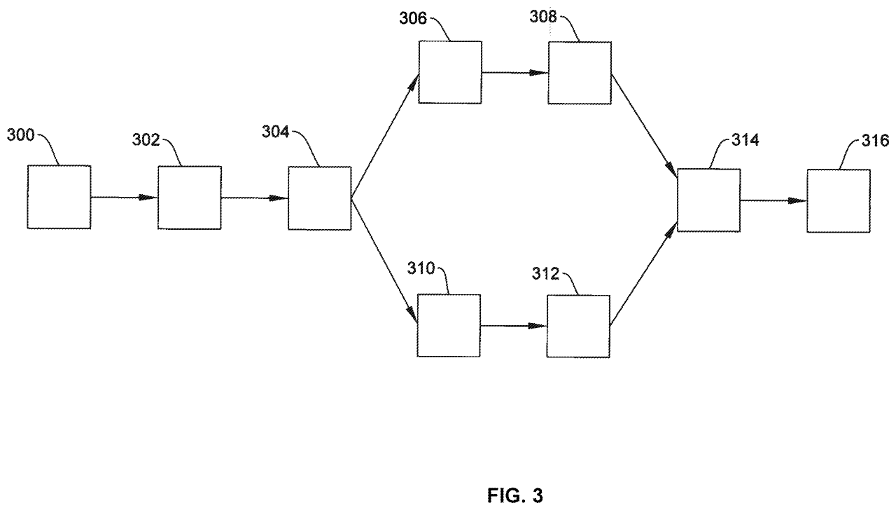

A schematic illustration of a method for identifying activation markers indicating activation of T cell receptors for a predetermined type of antigen (for example a neoantigen such as a personalized neoantigen or a shared neoantigen, inclusive of a neoantigen selected by a model whose parameters are adjusted using machine learning) is shown in FIG. 3. Samples are processed 300 to obtain a mixture containing a plurality of different T cells. The mixture can comprise, for example, PBMCs, such as PBMCs obtained by processing leukapheresis samples from healthy donors to remove cells positive for one or more of CD45RO, CD14, CD15, CD16, CD19, CD25, CD34, CD36, CD57, CD123, anti-HLA-DR, CD235a (Glycophorin A), CD244, and CD4. The plurality of different T cells can comprise, for example, naïve CD8⁺ T cells from healthy donors. The mixture is enriched 302 for the naïve CD8⁺ T cells by incubating with P-loaded MHC proteins (which P is the predetermined type of antigen) followed by isolating T cells bound thereto. The P-loaded MHC proteins can be provided, for example, in the form of magnetically-labeled multimers (to facilitate isolation by magnetic separation) or fluorescently-labeled multimers (to facilitate isolation via fluorescence flow cytometry). The isolated T cells are expanded 304, for example by polyclonal expansion, and partitioned into first and second T cell populations. The first T cell population is assessed 306 for P-binding T cells by incubating with P-loaded MHC proteins followed by isolating P-binding T cells bound thereto and sequencing 308 T cell receptors for the isolated P-binding T cells at a single cell level. The second T cell population is incubated 310 with cells presenting the predetermined type of antigen and at least a first activation marker (for example CD137 and/or a secreted molecule indicative of activation such as those disclosed herein, for example interferon gamma) measured to determine which members of the second T cell population are activated, followed by determining 312 the genetic expression profiles (for example by transcriptome analysis) and sequences of the T cell receptors for the second T cell population at a single cell level. The second T cell population is analyzed 314 by (a) partitioning (figuratively) the second T cell population into a plurality of T cell clusters; (b) identifying P-binding clusters within the plurality of T cell clusters by comparing the sequences of the T cell receptors for the second T cell population with the T cell receptor sequences of the P-binding T cells for the first T cell population; and (c) detecting which of the identified P-binding clusters contain at least a threshold number of cells presenting the at least the first activation marker. Genetic expression profiles for the detected P-binding clusters are evaluated 316 to identify further activation markers characteristic of P-activation of T cells.

FIG. 3 describes certain exemplary embodiments, but other variations fall within scope of the disclosure. In certain embodiments, for example, the PBMCs can be obtained from whole blood samples. In certain embodiments, for example, the plurality of different T cells can comprise CD-4⁺ T cells and the mixture can be enriched for the CD-4⁺ T cells. In certain embodiments, for example, the plurality of different T cells can comprise memory T cells and the mixture can be enriched for the memory T cells. In certain embodiments, for example, the processing can comprise removal of a different panel of biomarkers than shown depending on the desired composition of T cells for enrichment. In certain embodiments, for example, rather than forming clusters based on the detection of the at least the first activation marker, clusters can be formed based on similarity of T cell receptor sequences (for example clusters of T cells having at least portions of T cell receptor sequences characterized by sequence identities above a predetermined threshold). In certain embodiments, for example, the selected method of clustering can not depend on the at least the first activation marker and measurement of the at least the first activation marker omitted.

The disclosure also provides compositions that include one or more of the ingredients of the methods described herein. For example, in one embodiment, provided herein is a composition that includes an artificial T cell receptor selective to a predetermined type of antigen, at least a portion of a CDR3 region selected by analyzing a mixture of natural T cells to identify antigen-binding T cells and antigen-activated T cells for the predetermined type of antigen; and identifying at least a portion of at least one T cell receptor sequence shared by at least one of the antigen-binding T cells and at least one of the antigen-activated T cells, the at least a portion of at least one T cell receptor sequence containing the at least a portion of the CDR3 region; and a T cell receptor fragment. In another embodiment, provided herein is a composition that includes a P-binding T cell having at least one activation marker. In yet another embodiment, provided herein is a composition that includes a T cell receptor identified using the methods provided herein. In a further embodiment, provided herein is a composition that includes a T cell receptor clonotypes identified using the methods provided herein.

Also provided herein is a kit comprising one or more of the ingredients of the methods and compositions described herein. For example, in one embodiment, a kit comprises a predetermined type of antigen. In another embodiments, a kit comprises an assay for identifying an activation marker. In a further embodiment, the kit comprises an artificial T cell receptor selective to a predetermined type of antigen. In some embodiments, the kit includes a T cell receptor clonotype. It is further understood that the kits encompassed herein can be used in any of the methods disclosed herein.

### INCORPORATION BY REFERENCE

Without limitation, the following documents are hereby incorporated, in their entirety, by reference: U.S. Patent Application Publication Nos. 2017/0212984; 2017/0192011; 2017/0003288; U.S. Pat. Nos. 10,055,540; 10,066,265; International Patent Application Publication Nos. WO 2018/175585; WO 2018/165475; WO 2018/085453; WO 2017/075141; WO 2015/106151; European Patent No. EP 2327763; European Patent Application No. EP 2327763; Alanio, C. et al., “Enumeration of human-antigen-specific CD8+ T cells reveals conserved precursor frequencies,” Blood 115:18 (2010) 3718-3725; Moon, J. J. et al., “Naïve CD4+ T cell frequencies varies for different epitopes and predicts repertoire diversity and response magnitude,” Immunity 27:2 (August 2007) 203-213; Rius, C. et al., “Peptide-MHC Class I Tetramers Can Fail to Detect Relevant Functional T Cell Clonotypes and Underestimate Antigen-Reactive T Cell Populations,” J. Immunology 200 (2018) 2263-2279; Aleksic, M. et al., “Different affinity windows for virus and cancer-specific T-cell receptors—implications for therapeutic strategies,” European J. Immunology 42:12 (December 2012) 3174-3179; Dimopoulos, N. et al., “Combining MHC tetramer and intracellular cytokine staining for CD8+ T cells to reveal antigenic epitopes naturally presented on tumor cells,” J. Immunological Methods 340 (2009) 90-94; Kao H. et al., “A New Strategy for Tumor Antigen Discovery Based on in Vitro Priming of Naïve T Cells with Dendritic Cells,” Clinical Cancer Research 7 (2001) 773s-780s; Glanville, J. et al. “Identifying specificity groups in the T cell receptor repertoire,” Nature 547:7661 (2017) 94-98; Bulik-Sullivan, B. et al., “Deep learning using tumor HLA peptide mass spectrometry datasets improves neoantigen identification,” Nature Biotechnology, AOP (Dec. 11, 2018) 1-14; De Simone, D., “Single Cell T Cell Receptor Sequencing: Techniques and Future Challenges,” Frontiers In Immunology 9 (2018) Article 1638, 7 pages; Bossi, G., et al., “Examining the presentation of tumor-associated antigens on peptide-pulsed T2 cells” OncoImmunology 2:11 (2013) e26840-1 to e26840-6; Purbhoo, M. A., et al., “Quantifying and Imaging NY-ESO-1/LAGE-1-Derived Epitopes on Tumor Cells Using High Affinity T Cell Receptors” J Immunology 176 (2006) 7308-7316, Rosati et al., “Overview of methodologies for T-cell receptor repertoire analysis” BMC Biotechnology (2017) 17(1):61; Mahe, E. et al., “T cell clonality assessment: past, present and future” J Clin Pathol. (2018) March; 71(3):195-200; and Bagaev D V, et al. “VDJdb in 2019: database extension, new analysis infrastructure and a T-cell receptor motif compendium,” Nucleic Acids Res. 2020 Jan. 8; 48(D1):D1057-D1062 (collectively, the “INCORPORATED REFERENCES”).

### EXAMPLES

### Example 1 Identification of Antigen-Binding and Antigen-Activated T Cell Receptors for ASSLPTTMNY (SEQ ID NO: 1) Specific T Cells

This example illustrates that T cell receptor clonotypes present on antigen-specific and functional T cells were able to be successfully identified and selected using ASSLPTTMNY (SEQ ID NO: 1) as an exemplary antigen, and HLA-A*0101 as an exemplary gene encoding a class I MHC molecule. Although the example provided herein is illustrated using ASSLPTTMNY (SEQ ID NO: 1) as an exemplary antigen, it is understood that the method can be performed using any antigen that is less than 50 amino acids in length.

Peripheral blood mononuclear cells (PBMCs) were obtained from leukapheresis samples from HLA-A*0101-matched healthy donors. The PBMCs were cleared of cells positive for CD45RO, CD14, CD15, CD16, CD19, CD25, CD34, CD36, CD57, CD123, anti-HLA-DR, CD235a (Glycophorin A), CD244, and CD4 by exposure to biotinylated antibodies and magnetically labeled with streptavidin-coated microbeads, followed by magnetic sorting. Naïve CD8⁺ T cells obtained from the cleared PBMCs were stained with live/dead and lineage markers and isolated by passing through a fluorescence flow cytometry cell sorter. The naïve CD8⁺ T cells were polyclonally expanded to obtain the T cell sample. The T cell sample was then divided into three portions for identification of antigen binding and antigen-activated T cell receptors, and the assays were performed in duplicate.

To determine T cell receptor sequences that appeared in antigen-binding T cells, an Antigen-MHC test was preformed using ASSLPTTMNY (SEQ ID NO: 1) as the exemplary antigen. A first portion of the T cell sample was stained with fluorescent reporter-labeled antigen-MHC protein tetramers and passed through a fluorescence flow cytometry cell sorter. T cells that stained positive for the fluorescent reporter indicated antigen-binding.

In parallel, T cell activation following exposure to the exemplary antigen ASSLPTTMNY (SEQ ID NO: 1) was determined by separately measuring CD137 expression in one portion of cells, and IFN-γ secretion in another portion of cells. CD137, a member of the tumor necrosis factor receptor (TNFR) family, has been used successfully to identify antigen-reactive cells in both the CD4+ and CD8+ T cell compartments. IFN-γ secretion was used as an exemplary cytokine that is indicative of a T cell functional response.

To detect CD137 expression, a portion of the T cell sample was stimulated overnight with autologous PBMCs pulsed with 10 μM of the exemplary ASSLPTTMNY (SEQ ID NO: 1) antigen, stained with magnetically labelled CD137 antibody and isolated by magnetic separation. Cells that stained positive for CD137 indicated T cell activation in response to the specific antigen.

To detect IFN-γ secretion, another portion of the T cell sample, was stimulated overnight with autologous PBMCs pulsed with 10 μM of the antigen and assayed using Miltenyi IFN-γ Secretion Assay to isolate cells expressing IFN-γ. Cells that secreted IFN-γ indicated T cell activation in response to the specific antigen.

Following each of the different assays, positive hits were sequenced to determine T cell receptor sequences. The T cells were sequenced at single cell level using 10× Genomics single cell resolution paired immune TCR profiling. Sequencing reads were tagged with chromium cellular barcodes and unique molecular identifiers and frequencies of complete T cell receptor sequences determined.

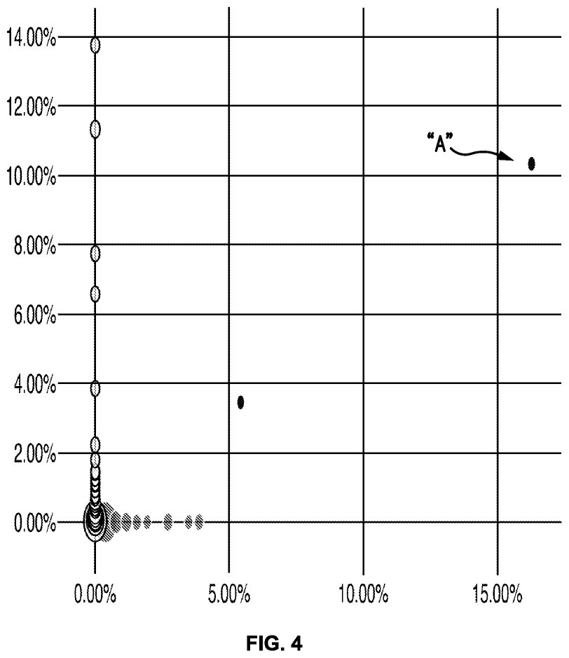

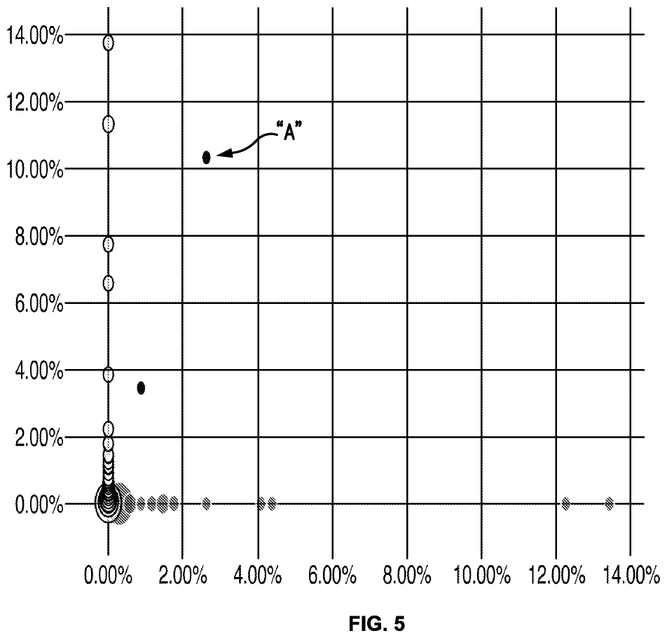

Comparison between T cell receptor sequences of CD137⁺ T cells and the T cells that bound the antigen (FIG. 4), as well as comparison between the T cell receptor sequences of IFN-γ secreting T cells and the T cells that bound the antigen (FIG. 5), demonstrated that the T cell receptor sequence could be identified for T cells that exhibited a high frequency of both antigen-binding and T cell activation. In addition, the results indicated that the T cell receptor sequence (Reference “A”) was able to be identified using both the CD137 assay, and the IFN-γ secreting assay (Table 2).

The sequence of Reference “A” revealed an alpha variable region having a peptide sequence of

(SEQ ID NO: 3)
MEKNPLAAPLLILWFHLDCVSSILNVEQSPQSLHVQEGDSTNFTCSFPS
SNFYALHVVYRWETAKSPEALFVMTLNGDEKKKGRISATLNTKEGYSYL
YIKGSQPEDSATYLCASPVDRGSTLGRLYFGRGTQLTVW,

and a beta variable region of

(SEQ ID NO: 5)
MGCRLLCCAVLCLLGAVPMETGVTQTPRHLVMGMTNKKSLKCEQHLGHN
AMYVVYKQSAKKPLELMFVYNFKEQTENNSVPSRFSPECPNSSHLFLHL
HTLQPEDSALYLCASSQVGTGSYEQYFGPGTRLTVT.

The TCR alpha and beta chains possess three hypervariable regions termed complementarity determining regions (CDR1, 2 and 3). CDR3 is responsible for recognizing and binding to processed antigen peptides, and leads to the clonal expansion of T cells. Sequencing of the T-cell receptor alpha chain VJ region and T-cell receptor beta chain VJ region also revealed the CDR3 for each the TCR alpha and beta chains (underlined peptides). Specifically, the CDR3 region of the alpha variable region for Reference “A” had a peptide sequence of CASPVDRGSTLGRLYF (SEQ ID NO: 21), and the CDR3 region of the beta variable region for Reference “A” had a peptide sequence of CASSQVGTGSYEQYF (SEQ ID NO: 22).

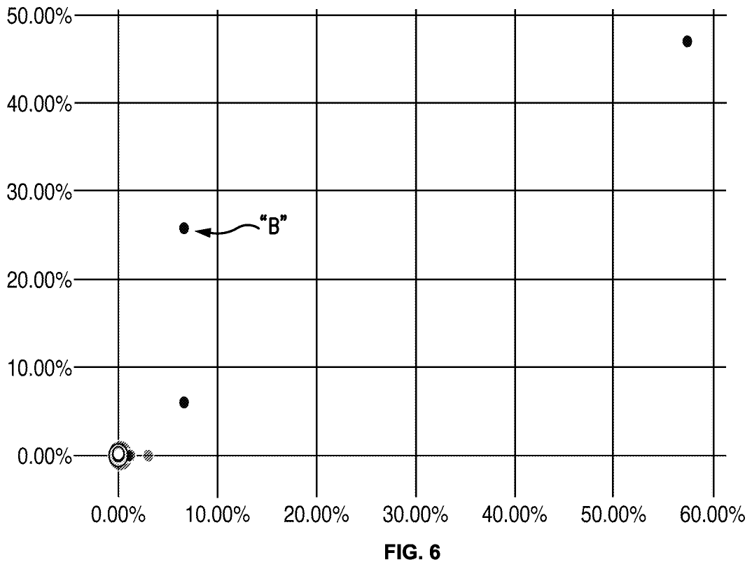

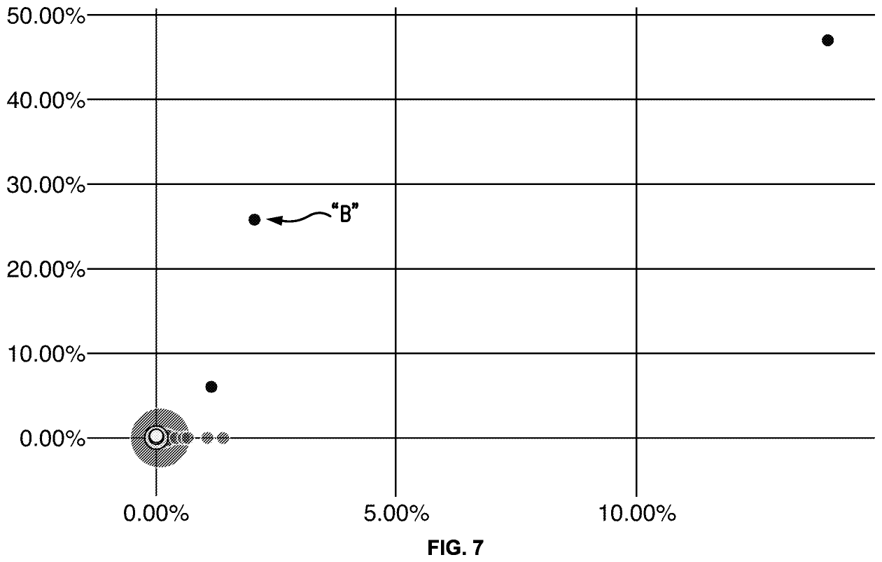

Similarly, in a second, independent experiment using the same exemplary antigen, ASSLPTTMNY (SEQ ID NO: 1), comparison between the T cell receptor sequences of CD137⁺ T cells and the T cells that bound the exemplary antigen (FIG. 6), as well as comparison between the T cell receptor sequences of IFN-γ secreting T cells and the T cells that bound the exemplary antigen (FIG. 7), indicated that the T cell receptor sequence for T cells with the highest frequency for both antigen-binding and CD137 expression or antigen-binding and IFN-γ secretion shared a common T cell receptor sequence (Reference “B”) (Table 2).

Sequencing of the T cell receptor sequence (Reference “B”) revealed an T-cell receptor alpha chain VJ region having a peptide sequence of

(SEQ ID NO: 4)
MLTASLLRAVIASICVVSSMAQKVTQAQTEISVVEKEDVTLDCVYETRD
TTYYLFVVYKQPPSGELVFLIRRNSFDEQNEISGRYSWNFQKSTSSFNF
TITASQVVDSAVYFCALSEARQYSGAGSYQLTFGKGTKLSVI,

and a T-cell receptor beta chain VJ region having a peptide sequence of

(SEQ ID NO: 6)
MSNQVLCCVVLCFLGANTVDGGITQSPKYLFRKEGQNVTLSCEQNLNHD
AMYVVYRQDPGQGLRLIYYSQIVNDFQKGDIAEGYSVSREKKESFPLTV
TSAQKNPTAFYLCASSLEWGPYEQYFGPGTRLTVT.

The CDR3 region of the alpha variable region for Reference “B” had a peptide sequence of CALSEARQYSGAGSYQLTF (SEQ ID NO: 23), and the CDR3 region of the beta variable region for Reference “B” had a peptide sequence of CASSLEWGPYEQYF (SEQ ID NO: 24).

Taken together, these results illustrate that T cell receptor clonotypes present on antigen-specific and functional T cells can be successfully selected, and represents a novel approach towards development of T cell lines that are therapeutically effective at physiologically relevant concentrations of an antigen.

### Example 2: Identification of Antigen-Binding and Antigen-Activated T Cell Receptors for HSEVGLPVY (SEQ ID NO: 2) Specific T Cells

This example illustrates that T cell receptor clonotypes present on antigen-specific and functional T cells were able to be successfully identified and selected using HSEVGLPVY (SEQ ID NO: 2) as another exemplary antigen, in combination with HLA-A*0101 as an exemplary MHC Class I encoding gene.

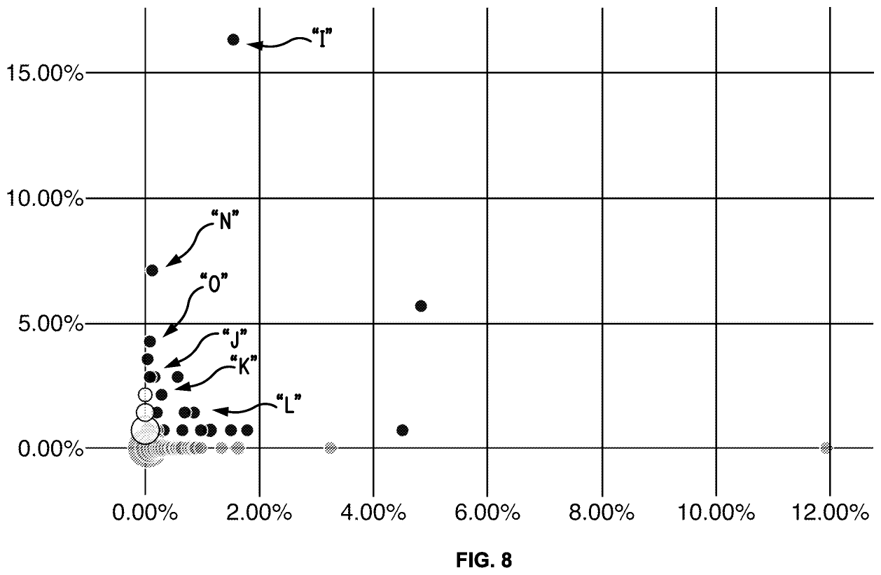

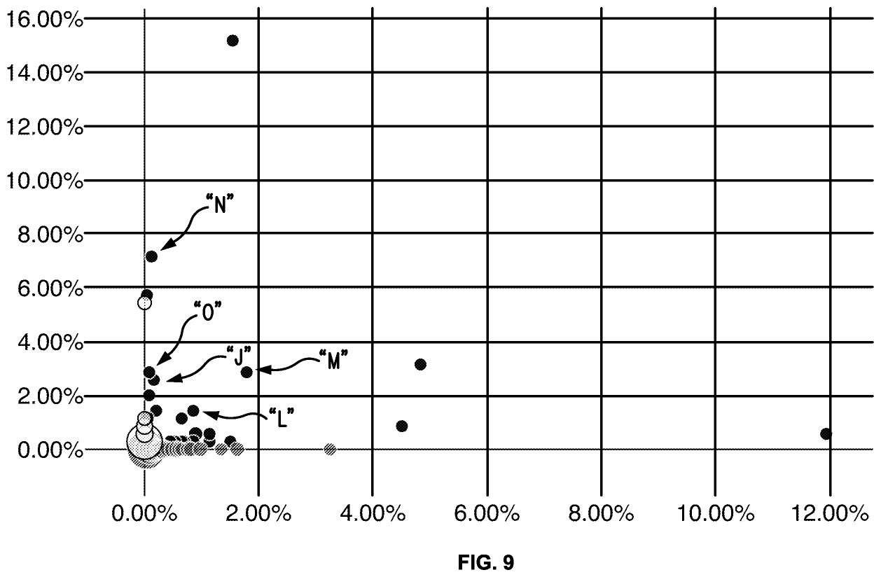

T cell samples were apportioned and separately tested for antigen binding and T cell activation using the CD137 expression assay, as described above in Example I. Positive hits were sequenced to determine T cell receptor sequences. T cell receptor sequences appearing in both antigen-binding T cells and antigen-activated T cells were noted (Table 3). FIG. 8 and FIG. 9 show the results from the first and second of the three replicates of the CD137 test, respectively.

As shown in Table 3, the peptide sequences for the T-cell receptor alpha chain VJ regions (SEQ ID NO: 7-SEQ ID NO: 13) and the peptide sequences for the T-cell receptor beta chain VJ regions (SEQ ID NO: 14-SEQ ID NO: 20) were determined for each of the T cell receptor sequence references (“I”-“I”). Notably, reference “H”, “I”, “J”, and “L” were among the T cell receptor sequences appearing in both antigen-binding T cells and antigen-activated T cells in at least two of the replicates of the CD137 test (FIG. 8 and FIG. 9).

In addition, the CDR3 regions for each of the identified T cell receptor sequences were determined (see text in Table 3 that is in bold and underlined). Specifically, the CDR3 region of the alpha variable region for Reference “I” had a peptide sequence of CAENSGGYQKVTF (SEQ ID NO: 25), and the CDR3 region of the beta variable region for Reference “I” had a peptide sequence of CASSVGDHTIYF (SEQ ID NO: 26). The CDR3 region of the alpha variable region for Reference “J” had a peptide sequence of CAMREGYRDDKIIF (SEQ ID NO: 27), and the CDR3 region of the beta variable region for Reference “J” had a peptide sequence of CASSFSSGGAHEQFF (SEQ ID NO: 28). The CDR3 region of the alpha variable region for Reference “K” had a peptide sequence of CAVNDYKLSF (SEQ ID NO: 29), and the CDR3 region of the beta variable region for Reference “K” had a peptide sequence of CASSIGWNYEQYF (SEQ ID NO: 30). The CDR3 region of the alpha variable region for Reference “L” had a peptide sequence of CILPNAGNMLTF (SEQ ID NO: 31), and the CDR3 region of the beta variable region for Reference “L” had a peptide sequence of CATRGTGTQPQHF (SEQ ID NO: 32). The CDR3 region of the alpha variable region for Reference “M” had a peptide sequence of CAGPREYGNKLVF (SEQ ID NO: 33), and the CDR3 region of the beta variable region for Reference “M” had a peptide sequence of CASSVGGQGEVVQYF (SEQ ID NO: 34). The CDR3 region of the alpha variable region for Reference “N” had a peptide sequence of CATDGKRVTGGGNKLTF (SEQ ID NO: 35), and the CDR3 region of the beta variable region for Reference “N” had a peptide sequence of CASSLWRTGELFF (SEQ ID NO: 36). The CDR3 region of the alpha variable region for Reference “0” had a peptide sequence of CADAPGSSYKLIF (SEQ ID NO: 37), and the CDR3 region of the beta variable region for Reference “0” had a peptide sequence of CASSQVPHEQYF (SEQ ID NO: 38).

Taken together, these results further illustrate that T cell receptor clonotypes present on antigen-specific and functional T cells can be successfully selected using the methods provided herein. In addition, the results demonstrate that the T cell receptor sequences identified by this method are reproducible.

### Example 3: Identification of Antigen-Binding and Antigen-Activated T Cell Receptors

This example demonstrates that the methods provided herein can also identify T cell receptors capable of sensing antigens presented by class II molecules of the major histocompatibility complex (MHC).

Peripheral blood mononuclear cells (PBMCs) can be obtained from leukapheresis samples from a Class II HLA, such as for example a HLA-DRB*101:01, matched healthy donor. The PBMCs can be cleared of cells positive for CD45RO, CD14, CD15, CD16, CD19, CD25, CD34, CD36, CD57, CD123, CD235a (Glycophorin A), CD244, and CD8 by exposure to biotinylated antibodies and magnetically labeled with streptavidin-coated microbeads, followed by magnetic sorting. Naïve CD4⁺ T cells obtained from the cleared PBMCs can be stained with live/dead and lineage markers and isolated by passing through a fluorescence flow cytometry cell sorter. The naïve CD4⁺ T cells can be polyclonally expanded to obtain the T cell sample.

T cell samples can be apportioned and separately tested for antigen binding and T cell activation using the CD137 expression assay, as described above in Example I. Positive hits can be sequenced to determine T cell receptor sequences. T cell receptor sequences appearing in both antigen-binding T cells and antigen-activated T cells can be determined, and the specific peptide sequences for the T-cell receptor alpha chain VJ regions and the peptide sequences for the T cell receptor beta chain VJ regions can be determined for each of the T cell receptor sequence references. In addition, the CDR3 regions for each of the identified T cell receptor sequences can be determined.

This example demonstrates that T cell receptor sequences capable of interacting with antigens presented by class II MHC molecules.

TABLE 1
Experimental parameters.

| | | | | T Cell | T Cell |
|---|---|---|---|---|---|
| | T Cell | | | Binding | Activation |
| Example¹ | Sample² | Antigen | MHC | Test | Test(s) |
| 1 | Naïve | ASSLPT | HLA- | Antigen- | CD137 |
| | CD8⁺ | TMNY | A* | MHC | Test⁴ |
| | | (SEQ ID | 0101 | Test³ | Interferon |
| | | NO: 1) | | | Gamma |
| | | | | | Test⁵ |
| 2 | Naïve | HSEVG | HLA- | Antigen- | CD137 |
| | CD8⁺ | LPVY | A* | MHC | Test⁶ |
| | | (SEQ ID | 0101 | Test | |
| | | NO: 2) | | | |

¹Example 1 was performed in duplicate.
²Peripheral blood mononuclear cells were obtained from leukapheresis samples from HLA-A*0101-matched healthy donors. The PBMCs were cleared of cells positive for CD45RO, CD14, CD15, CD16, CD19, CD25, CD34, CD36, CD57, CD123, anti-HLA-DR, CD235a (Glycophorin A), CD244, and CD4 by exposure to biotinylated antibodies and magnetically labeled with streptavidin
coated microbeads, followed by magnetic sorting. Naïve CD8⁺ T cells obtained from the cleared PBMCs were stained with live/dead and lineage markers and isolated by passing through a fluorescence flow cytometry cell sorter. The naïve CD8⁺ T cells were polyclonally expanded to obtain the T cell sample.
³Antigen-MHC Test: A first portion of the T cell sample was stained with fluorescent reporter-labeled antigen-MHC protein tetramers and passed through a fluorescence flow cytometry cell sorter.
⁴CD137 Test: A second portion of the T cell sample was stimulated overnight with autologous PBMCs pulsed with 10 μM of the antigen, stained with magnetically labelled CD137 antibody and isolated by magnetic separation.
⁵Interferon Gamma Test: A third portion of the T cell sample was stimulated overnight with autologous PBMCs pulsed with 10 μM of the antigen and assayed using Miltenyi IFN-y Secretion Assay to isolate cells expressing Interferon Gamma.
⁶In Example 2, the CD137 Test was replicated on three subsamples of the first portion. FIGS. 8-9 depict the first two replicates. The third replicate is not shown.

TABLE 2
T cell receptors shared between antigen-binding and antigen-activated T cells in Example 1.¹

| | | | | | | | | T Cell Receptor Sequences² | | | |
|---|---|---|---|---|---|---|---|---|---|---|---|
| | Annotated Sequences | | | | | | | Alpha | CDR3 | Beta | CDR3 |
| Ref | TRAV | TRAJ | TRAC | TRBV | TRBD | TRBJ | TRBC | VJ | alpha | VJ | beta |
| A | TRAV | TRAJ | TRAC | TRBV | TRBD | TRBJ | TRBC | MEKN | CASP | MGCR | CASS |
| | 24 | 18 | | 4-2 | 1 | 2-7 | 2 | PLAA | VDRG | LLCC | QVGT |
| | | | | | | | | PLLI | STLG | AVLC | GSYE |
| | | | | | | | | LWFH | RLYF | LLGA | QYF |
| | | | | | | | | LDCV | (SEQ | VPME | (SEQ |
| | | | | | | | | SSIL | ID | TGVT | ID |
| | | | | | | | | NVEQ | NO: | QTPR | NO: |
| | | | | | | | | SPQS | 21) | HLVM | 22) |
| | | | | | | | | LHVQ | | GMTN | |
| | | | | | | | | EGDS | | KKSL | |
| | | | | | | | | TNFT | | KCEQ | |
| | | | | | | | | CSFP | | HLGH | |
| | | | | | | | | SSNF | | NAMY | |
| | | | | | | | | YALH | | WYKQ | |
| | | | | | | | | WYRW | | SAKK | |
| | | | | | | | | ETAK | | PLEL | |
| | | | | | | | | SPEA | | MFVY | |
| | | | | | | | | LFVM | | NFKE | |
| | | | | | | | | TLNG | | QTEN | |
| | | | | | | | | DEKK | | NSVP | |
| | | | | | | | | KGRI | | SRFS | |
| | | | | | | | | SATL | | PECP | |
| | | | | | | | | NTKE | | NSSH | |
| | | | | | | | | GYSY | | LFLH | |
| | | | | | | | | LYIK | | LHTL | |
| | | | | | | | | GSQP | | QPED | |
| | | | | | | | | EDSA | | SALY | |
| | | | | | | | | TYL | | L | |
| | | | | | | | | [symbol] | | [symbol] | |
| | | | | | | | | [symbol] G | | [symbol] | |
| | | | | | | | | RGTQ | | GPGT | |
| | | | | | | | | LTVW | | RLTV | |
| | | | | | | | | (SEQ | | T | |
| | | | | | | | | ID | | (SEQ | |
| | | | | | | | | NO: | | ID | |
| | | | | | | | | 3) | | NO: | |
| | | | | | | | | | | 5) | |
| B | TRAV | TRAJ | TRAC | TRBV | TRBD | TRBJ | TRBC | MLTA | CALS | MSNQ | CASS |
| | 19 | 28 | | 19 | 2 | 2-7 | 2 | SLLR | EARQ | VLCC | LEWG |
| | | | | | | | | AVIA | YSGA | VVLC | PYEQ |
| | | | | | | | | SICV | GSYQ | FLGA | YF |
| | | | | | | | | VSSM | LTF | NTVD | (SEQ |
| | | | | | | | | AQKV | (SEQ | GGIT | ID |
| | | | | | | | | TQAQ | ID | QSPK | NO: |
| | | | | | | | | TEIS | NO: | YLFR | 24) |
| | | | | | | | | VVEK | 23) | KEGQ | |
| | | | | | | | | EDVT | | NVTL | |
| | | | | | | | | LDCV | | SCEQ | |
| | | | | | | | | YETR | | NLNH | |
| | | | | | | | | DTTY | | DAMY | |
| | | | | | | | | YLFW | | WYRQ | |
| | | | | | | | | YKQP | | DPGQ | |
| | | | | | | | | PSGE | | GLRL | |
| | | | | | | | | LVFL | | IYYS | |
| | | | | | | | | IRRN | | QIVN | |
| | | | | | | | | SFDE | | DFQK | |
| | | | | | | | | QNEI | | GDIA | |
| | | | | | | | | SGRY | | EGYS | |
| | | | | | | | | SWNF | | VSRE | |
| | | | | | | | | QKST | | KKES | |
| | | | | | | | | SSFN | | FPLT | |
| | | | | | | | | FTIT | | VTSA | |
| | | | | | | | | ASQW | | QKNP | |
| | | | | | | | | DSAV | | TAFY | |
| | | | | | | | | YF | | L | |
| | | | | | | | | [symbol] | | [symbol] | |
| | | | | | | | | [symbol] | | [symbol] G | |
| | | | | | | | | [symbol] | | PGTR | |
| | | | | | | | | GKG | | LTVT | |
| | | | | | | | | TKLS | | (SEQ | |
| | | | | | | | | VI | | ID | |
| | | | | | | | | (SEQ | | NO: | |
| | | | | | | | | ID | | 6) | |
| | | | | | | | | NO: | | | |
| | | | | | | | | 4) | | | |
| C | TRAV | TRAJ | TRAC | TRBV | TRBD | TRBJ | TRBC | MISL | CVVP | MGSR | CASS |
| | 12-1 | 12 | | 5-1 | 2 | 2-7 | 2 | RVLL | RMDS | LLCW | STGA |
| | | | | | | | | VILW | SYKL | VLLC | RRSR |
| | | | | | | | | LQLS | IF | LLGA | EQYF |
| | | | | | | | | WVWS | (SEQ | GPVK | (SEQ |
| | | | | | | | | QRKE | ID | AGVT | ID |
| | | | | | | | | VEQD | NO: | QTPR | NO: |
| | | | | | | | | PGPF | 51) | YLIK | 52) |
| | | | | | | | | NVPE | | TRGQ | |
| | | | | | | | | GATV | | QVTL | |
| | | | | | | | | AFNC | | SCSP | |
| | | | | | | | | TYSN | | ISGH | |
| | | | | | | | | SASQ | | RSVS | |
| | | | | | | | | SFFW | | WYQQ | |
| | | | | | | | | YRQD | | TPGQ | |
| | | | | | | | | CRKE | | GLQF | |
| | | | | | | | | PKLL | | LFEY | |
| | | | | | | | | MSVY | | FSET | |
| | | | | | | | | SSGN | | QRNK | |
| | | | | | | | | EDGR | | GNFP | |
| | | | | | | | | FTAQ | | GRFS | |
| | | | | | | | | LNRA | | GRQF | |
| | | | | | | | | SQYI | | SNSR | |
| | | | | | | | | SLLI | | SEMN | |
| | | | | | | | | RDSK | | VSTL | |
| | | | | | | | | LSDS | | ELGD | |
| | | | | | | | | ATYL | | SALY | |
| | | | | | | | | [symbol] | | L | |
| | | | | | | | | [symbol] | | [symbol] | |
| | | | | | | | | GSGT | | [symbol] | |
| | | | | | | | | RLLV | | GPGT | |
| | | | | | | | | RP | | RLTV | |
| | | | | | | | | (SEQ | | T | |
| | | | | | | | | ID | | (SEQ | |
| | | | | | | | | NO: | | ID | |
| | | | | | | | | 39) | | NO: | |
| | | | | | | | | | | 45) | |
| D | TRAV19 | TRAJ28 | TRAC | TRBV19 | TRBD2 | TRBJ2- | TRBC2 | MLTA | CALS | MSNQ | CASS |
| | | | | | | 7 | | SLLR | EARQ | VLCC | LEWG |
| | | | | | | | | AVIA | YSGA | WLCF | PYEQ |
| | | | | | | | | SICV | GSYQ | LGAN | YF |
| | | | | | | | | VSSM | LTF | TVDG | (SEQ |
| | | | | | | | | AQKV | (SEQ | GITQ | ID |
| | | | | | | | | TQAQ | I | SPKY | NO: |
| | | | | | | | | TEIS | DNO: | LFRK | 54) |
| | | | | | | | | WEKE | 53) | EGQN | |
| | | | | | | | | DVTL | | VTLS | |
| | | | | | | | | DCVY | | CEQN | |
| | | | | | | | | ETRD | | LNHD | |
| | | | | | | | | TTYY | | AMYW | |
| | | | | | | | | LFWY | | YRQD | |
| | | | | | | | | KQPP | | PGQG | |
| | | | | | | | | SGEL | | LRLI | |
| | | | | | | | | VFLI | | YYSQ | |
| | | | | | | | | RRNS | | IVND | |
| | | | | | | | | FDEQ | | FQKG | |
| | | | | | | | | NEIS | | DIAE | |
| | | | | | | | | GRYS | | GYSV | |
| | | | | | | | | WNFQ | | SREK | |
| | | | | | | | | KSTS | | KESF | |
| | | | | | | | | SFNF | | PLTV | |
| | | | | | | | | TITA | | TSAQ | |
| | | | | | | | | SQVV | | KNPT | |
| | | | | | | | | DSAV | | AFYL | |
| | | | | | | | | YF | | [symbol] | |
| | | | | | | | | [symbol] | | [symbol] | |
| | | | | | | | | [symbol] | | GPGT | |
| | | | | | | | | [symbol] | | RLTV | |
| | | | | | | | | GKGT | | T | |
| | | | | | | | | KLS | | (SEQ | |
| | | | | | | | | VIP | | ID | |
| | | | | | | | | (SEQ | | NO: | |
| | | | | | | | | ID | | 46) | |
| | | | | | | | | NO: | | | |
| | | | | | | | | 40) | | | |
| E | TRAV25 | TRAJ30 | TRAC | TRBV19 | TRBD2 | TRBJ2- | TRBC2 | MLLI | CAGQ | MSNQ | CASS |
| | | | | | | 7 | | TSML | GNRD | VLCC | LEWG |
| | | | | | | | | VLWM | DKII | WLCF | PYEQ |
| | | | | | | | | QLSQ | F | LGAN | YF |
| | | | | | | | | VNGQ | (SEQ | TVDG | (SEQ |
| | | | | | | | | QVMQ | ID | GITQ | ID |
| | | | | | | | | IPQY | NO: | SPKY | NO: |
| | | | | | | | | QHVQ | 55) | LFRK | 56) |
| | | | | | | | | EGED | | EGQN | |
| | | | | | | | | FTTY | | VTLS | |
| | | | | | | | | CNSS | | CEQN | |
| | | | | | | | | TTLS | | LNHD | |
| | | | | | | | | NIQW | | AMYW | |
| | | | | | | | | YKQR | | YRQD | |
| | | | | | | | | PGGH | | PGQG | |
| | | | | | | | | PVFL | | LRLI | |
| | | | | | | | | IQLV | | YYSQ | |
| | | | | | | | | KSGE | | IVND | |
| | | | | | | | | VKKQ | | FQKG | |
| | | | | | | | | KRLT | | DIAE | |
| | | | | | | | | FQFG | | GYSV | |
| | | | | | | | | EAKK | | SREK | |
| | | | | | | | | NSSL | | KESF | |
| | | | | | | | | HITA | | PLTV | |
| | | | | | | | | TQTT | | TSAQ | |
| | | | | | | | | DVGT | | KNPT | |
| | | | | | | | | YF | | AFYL | |
| | | | | | | | | [symbol] | | [symbol] | |
| | | | | | | | | [symbol] | | [symbol] | |
| | | | | | | | | GKGT | | GP | |
| | | | | | | | | RLHI | | GTRL | |
| | | | | | | | | LP | | TVT | |
| | | | | | | | | (SEQ | | (SEQ | |
| | | | | | | | | ID | | ID | |
| | | | | | | | | NO: | | NO: | |
| | | | | | | | | 41) | | 47) | |
| F | TRAV12 | TRAJ31 | TRAC | TRBV5- | TRBD2 | TRBJ2- | TRBC2 | MKSL | CAVK | MGSR | CASS |
| | -2 | | | 1 | | 7 | | RVLL | DNNA | LLCW | LSSG |
| | | | | | | | | VILW | RLMF | VLLC | LYEQ |
| | | | | | | | | LQLS | (SEQ | LLGA | YF |
| | | | | | | | | WVWS | ID | GPVK | (SEQ |
| | | | | | | | | QQKE | NO: | AGVT | ID |
| | | | | | | | | VEQN | 57) | QTPR | NO: |
| | | | | | | | | SGPL | | YLIK | 58) |
| | | | | | | | | SVPE | | TRGQ | |
| | | | | | | | | GAIA | | QVTL | |
| | | | | | | | | SLNC | | SCSP | |
| | | | | | | | | TYSD | | ISGH | |
| | | | | | | | | RGSQ | | RSVS | |
| | | | | | | | | SFFW | | WYQQ | |
| | | | | | | | | YRQY | | TPGQ | |
| | | | | | | | | SGKS | | GLQF | |
| | | | | | | | | PELI | | LFEY | |
| | | | | | | | | MFIY | | FSET | |
| | | | | | | | | SNGD | | QRNK | |
| | | | | | | | | KEDG | | GNFP | |
| | | | | | | | | RFTA | | GRFS | |
| | | | | | | | | QLNK | | GRQF | |
| | | | | | | | | ASQY | | SNSR | |
| | | | | | | | | VSLL | | SEMN | |
| | | | | | | | | IRDS | | VSTL | |
| | | | | | | | | QPSD | | ELGD | |
| | | | | | | | | SATY | | SALY | |
| | | | | | | | | L | | L | |
| | | | | | | | | [symbol] | | [symbol] | |
| | | | | | | | | [symbol] GDG | | [symbol] | |
| | | | | | | | | TQLW | | GPGT | |
| | | | | | | | | KP | | RLTV | |
| | | | | | | | | (SEQ | | T | |
| | | | | | | | | ID | | (SEQ | |
| | | | | | | | | NO: | | ID | |
| | | | | | | | | 42) | | NO: | |
| | | | | | | | | | | 48) | |
| G | TRAV17 | TRAJ48 | TRAC | TRBV5- | TRBD2 | TRBJ2- | TRBC1 | METL | CATA | MGSR | CASS |
| | | | | 1 | | 7 | | LGVS | VFNF | LLCW | SMTS |
| | | | | | | | | LVIL | GNEK | VLLC | GGPW |
| | | | | | | | | WLQL | LTF | LLGA | EQYF |
| | | | | | | | | ARVN | (SEQ | GPVK | |
| | | | | | | | | SQQG | ID | AGVT | (SEQ |
| | | | | | | | | EEDP | NO: | QTPR | ID |
| | | | | | | | | QALS | 59) | YLIK | NO |
| | | | | | | | | IQEG | | TRGQ | :60) |
| | | | | | | | | ENAT | | QVTL | |
| | | | | | | | | MNCS | | SCSP | |
| | | | | | | | | YKTS | | ISGH | |
| | | | | | | | | INNL | | RSVS | |
| | | | | | | | | QWYR | | WYQQ | |
| | | | | | | | | QNSG | | TPGQ | |
| | | | | | | | | RGLV | | GLQF | |
| | | | | | | | | HLIL | | LFEY | |
| | | | | | | | | IRSN | | FSET | |
| | | | | | | | | EREK | | QRNK | |
| | | | | | | | | HSGR | | GNFP | |
| | | | | | | | | LRVT | | GRFS | |
| | | | | | | | | LDTS | | GRQF | |
| | | | | | | | | KKSS | | SNSR | |
| | | | | | | | | SLLI | | SEMN | |
| | | | | | | | | TASR | | VSTL | |
| | | | | | | | | AADT | | ELGD | |
| | | | | | | | | ASYF | | SALY | |
| | | | | | | | | [symbol] | | L | |
| | | | | | | | | [symbol] | | [symbol] | |
| | | | | | | | | [symbol] G | | [symbol] | |
| | | | | | | | | TGTR | | GPGT | |
| | | | | | | | | LTII | | RLTV | |
| | | | | | | | | P | | T | |
| | | | | | | | | (SEQ | | (SEQ | |
| | | | | | | | | ID | | ID | |
| | | | | | | | | NO: | | NO: | |
| | | | | | | | | 43) | | 49) | |
| H | TRAV13 | TRAJ27 | TRAC | TRBV24 | TRBD2 | TRBJ2- | TRBC2 | MAGI | CAEN | MASL | CATS |
| | -2 | | | -1 | | 1 | | RALF | MGGA | LFFC | GGVA |
| | | | | | | | | MYLW | GKST | GAFY | GVRQ |
| | | | | | | | | LQLD | F | LLGT | FF |
| | | | | | | | | WVSR | (SEQ | GSMD | (SEQ |
| | | | | | | | | GESV | ID | ADVT | ID |
| | | | | | | | | GLHL | NO: | QTPR | NO: |
| | | | | | | | | PTLS | 61) | NRIT | 62) |
| | | | | | | | | VQEG | | KTGK | |
| | | | | | | | | DNSI | | RIML | |
| | | | | | | | | INCA | | ECSQ | |
| | | | | | | | | YSNS | | TKGH | |
| | | | | | | | | ASDY | | DRMY | |
| | | | | | | | | FIWY | | WYRQ | |
| | | | | | | | | KQES | | DPGL | |
| | | | | | | | | GKGP | | GLRL | |
| | | | | | | | | QFII | | IYYS | |
| | | | | | | | | DIRS | | FDVK | |
| | | | | | | | | NMDK | | DINK | |
| | | | | | | | | RQGQ | | GEIS | |
| | | | | | | | | RVTV | | DGYS | |
| | | | | | | | | LLNK | | VSRQ | |
| | | | | | | | | TVKH | | AQAK | |
| | | | | | | | | LSLQ | | FSLS | |
| | | | | | | | | IAAT | | LESA | |
| | | | | | | | | QPGD | | IPNQ | |
| | | | | | | | | SAVY | | TALY | |
| | | | | | | | | F [symbol] | | F [symbol] | |
| | | | | | | | | [symbol] | | [symbol] | |
| | | | | | | | | GD | | [symbol] G | |
| | | | | | | | | GTTL | | PGTR | |
| | | | | | | | | TVKP | | LTVL | |
| | | | | | | | | (SEQ | | (SEQ | |
| | | | | | | | | ID | | ID | |
| | | | | | | | | NO: | | NO: | |
| | | | | | | | | 44) | | 50) | |

⁷¹¹T cells were sequenced at single cell level using 10× Genomics single cell resolution paired immune TCR profiling. Sequencing reads were tagged with Chromium cellular barcodes and unique molecular identifiers and frequencies of complete T cell receptor sequences determined (see FIGS. 4-7). Alpha/beta chain pairs common to at least two antigen-binding T cells and at least two antigen-activated T cells are reported.
²CDR3 regions are underlined and in bold.

TABLE 3
T cell receptors shared between antigen-binding and antigen-activated T cells in Example 2.¹

| | | | | | | | | T Cell Receptor Sequences² | | | |
|---|---|---|---|---|---|---|---|---|---|---|---|
| | Annotated Sequences | | | | | | | Alpha | CDR3 | Beta | CDR3 |
| | TRAV | TRAJ | TRAC | TRBV | beta | TRBJ | TRBC | VJ | alpha | VJ | beta |
| Ref | TRAV13 | TRAJ13 | TRAC | TRBV9 | None | TRBJ1- | TRBC1 | MAGIRALFMYLW | CAENSG | MGFRLLCCVAFC | CASSVG |
| I | -2 | | | | | 3 | | LQLDWVSRGESV | GYQKVT | LLGAGPVDSGVT | DHTIYF |
| | | | | | | | | GLHLPTLSVQEG | F | QTPKHLITATGQ | (SEQ ID |
| | | | | | | | | DNSIINCAYSNSA | (SEQ ID | RVTLRCSPRSGD | NO: 26) |
| | | | | | | | | SDYFIWYKQESG | NO: 25) | LSVYWYQQSLD | |
| | | | | | | | | KGPQFIIDIRSNM | | QGLQFLIQYYNG | |
| | | | | | | | | DKRQGQRVTVLL | | EERAKGNILERFS | |
| | | | | | | | | NKTVKHLSLQIAA | | AQQFPDLHSELN | |
| | | | | | | | | TQPGDSAVYF [symbol] | | LSSLELGDSALYF | |
| | | | | | | | | [symbol] | | [symbol] | |
| | | | | | | | | GTGTKLQVI | | GEGSWLTW | |
| | | | | | | | | (SEQ ID | | (SEQ ID | |
| | | | | | | | | NO: 7) | | NO: 14) | |
| J | TRAV14 | TRAJ30 | TRAC | TRBV6- | TRBD2 | TRBJ2- | TRBC2 | MSLSSLLKWTA | CAMREG | MSISLLCCAAFPL | CASSFSS |
| | DV4 | | | 6 | | 1 | | SLWLGPGIAQKIT | YRDDKI | LWAGPVNAGVT | GGAHE |
| | | | | | | | | QTQPGMFVQEK | IF | QTPKFRILKIGQS | QFF |
| | | | | | | | | EAVTLDCTYDTS | (SEQ ID | MTLQCTQDMNH | (SEQ ID |
| | | | | | | | | DQSYGLFWYKQ | NO: | NYMYWYRQDPG | NO: |
| | | | | | | | | PSSGEMIFLIYQG | 27) | MGLKLIYYSVGA | 28) |
| | | | | | | | | SYDEQNATEGRY | | GITDKGEVPNGY | |
| | | | | | | | | SLNFQKARKSAN | | NVSRSTTEDFPL | |
| | | | | | | | | LVISASQLGDSA | | RLELAAPSQTSV | |
| | | | | | | | | MYF [symbol] | | YF [symbol] | |
| | | | | | | | | [symbol] GKGTRLH | | [symbol] GPGTRLT | |
| | | | | | | | | IL (SEQ ID | | VL | |
| | | | | | | | | NO: 8) | | (SEQ ID | |
| | | | | | | | | | | NO: 15) | |
| K | TRAV27 | TRAJ20 | TRAC | TRBV19 | None | TRBJ2- | TRBC2 | MVLKFSVSILWIQ | CAVND | MSNQVLCCWLC | CASSIG |
| | | | | | | 7 | | LAWVSTQLLEQS | YKLSF | FLGANTVDGGIT | WNYEQY |
| | | | | | | | | PQFLSIQEGENLT | (SEQ ID | QSPKYLFRKEGQ | F |
| | | | | | | | | VYCNSSSVFSSL | NO: 29) | NVTLSCEQNLNH | (SEQ ID |
| | | | | | | | | QWYRQEPGEGP | | DAMYWYRQDPG | NO: 30) |
| | | | | | | | | VLLVTWTGGEV | | QGLRLIYYSQIVN | |
| | | | | | | | | KKLKRLTFQFGD | | DFQKGDIAEGYS | |
| | | | | | | | | ARKDSSLHITAAQ | | VSREKKESFPLT | |
| | | | | | | | | PGDTGLYL [symbol] | | VTSAQKNPTAFY | |
| | | | | | | | | [symbol] GAGTTV | | L [symbol] | |
| | | | | | | | | TVR | | [symbol] GPGTRLTVT | |
| | | | | | | | | (SEQ ID | | (SEQ ID | |
| | | | | | | | | NO: 9) | | NO: 16) | |
| L | TRAV26 | TRAJ39 | TRAC | TRBV15 | TRBD1 | TRBJ1- | TRBC1 | MKLVTSITVLLSL | CILPNAG | MGPGLLHWMAL | CATRGT |
| | -2 | | | | | 5 | | GIMGDAKTTQPN | NMLTF | CLLGTGHGDAMV | GTQPQHF |
| | | | | | | | | SMESNEEEPVHL | (SEQ ID | IQNPRYQVTQFG | (SEQ ID |
| | | | | | | | | PCNHSTISGTDYI | NO: 31) | KPVTLSCSQTLN | NO: 32) |
| | | | | | | | | HWYRQLPSQGP | | HNVMYWYQQKS | |
| | | | | | | | | EYVIHGLTSNVN | | SQAPKLLFHYYD | |
| | | | | | | | | NRMASLAIAEDR | | KDFNNEADTPDN | |
| | | | | | | | | KSSTLILHRATLR | | FQSRRPNTSFCF | |
| | | | | | | | | DAAVYY [symbol] | | LDIRSPGLGDAA | |
| | | | | | | | | [symbol] GGGTRL | | MYL [symbol] | |
| | | | | | | | | MVK | | [symbol] GDGTRLSI | |
| | | | | | | | | (SEQ ID | | L (SEQ ID | |
| | | | | | | | | NO: 10) | | NO: 17) | |
| M | TRAV25 | TRAJ47 | TRAC | TRBV2 | TRBD1 | TRBJ2- | TRBC2 | MLLITSMLVLWM | CAGPREY | MDTWLVCWAIFS | CASSVGG |
| | | | | | | 7 | | QLSQVNGQQVM | GNKLVF | LLKAGLTEPEVT | QGEVVQYF |
| | | | | | | | | QIPQYQHVQEGE | (SEQ ID | QTPSHQVTQMG | (SEQ ID |
| | | | | | | | | DFTTYCNSSTTL | NO: 33) | QEVILRCVPISNH | NO: 34) |
| | | | | | | | | SNIQWYKQRPG | | LYFYWYRQILGQ | |
| | | | | | | | | GHPVFLIQLVKSG | | KVEFLVSFYNNEI | |
| | | | | | | | | EVKKQKRLTFQF | | SEKSEIFDDQFSV | |
| | | | | | | | | GEAKKNSSLHITA | | ERPDGSNFTLKIR | |
| | | | | | | | | TQTTDVGTYF [symbol] | | STKLEDSAMYF [symbol] | |
| | | | | | | | | [symbol] G | | [symbol] | |
| | | | | | | | | AGTILRVK | | [symbol] GPGTRLTVT | |
| | | | | | | | | (SEQ ID | | (SEQ ID | |
| | | | | | | | | NO: 11) | | NO: 18) | |
| N | TRAV17 | TRAJ10 | TRAC | TRBV11 | TRBD2 | TRBJ2- | TRBC2 | METLLGVSLVILW | CATDGK | MGTRLLCWAALC | CASSLWR |
| | | | | -2 | | 2 | | LQLARVNSQQGE | RVTGG | LLGAELTEAGVA | TGELFF |
| | | | | | | | | EDPQALSIQEGE | GNKLTF | QSPRYKIIEKRQS | (SEQ ID |
| | | | | | | | | NATMNCSYKTSI | (SEQ ID | VAFWCNPISGHA | NO: 36) |
| | | | | | | | | NNLQWYRQNSG | NO: 35) | TLYWYQQILGQG | |
| | | | | | | | | RGLVHLILIRSNE | | PKLLIQFQNNGV | |
| | | | | | | | | REKHSGRLRVTL | | VDDSQLPKDRFS | |
| | | | | | | | | DTSKKSSSLLITA | | AERLKGVDSTLKI | |
| | | | | | | | | SRAADTASYF [symbol] | | QPAKLEDSAVYL | |
| | | | | | | | | [symbol] | | [symbol] | |
| | | | | | | | | [symbol] GTGTQLKV | | [symbol] GEGSRLTVL | |
| | | | | | | | | E | | (SEQ ID | |
| | | | | | | | | (SEQ ID | | NO: 19) | |
| | | | | | | | | NO: 12) | | | |
| O | TRAV1- | TRAJ12 | TRAC | TRBV3- | None | TRBJ2- | TRBC2 | MWGAFLLYVSM | CADAPG | MGCRLLCCWFC | CASSQVP |
| | 1 | | | 1 | | 7 | | KMGGTAGQSLE | SSYKLIF | LLQAGPLDTAVS | HEQYF |
| | | | | | | | | QPSEVTAVEGAI | (SEQ ID | QTPKYLVTQMGN | (SEQ ID |
| | | | | | | | | VQINCTYQTSGF | NO: 37) | DKSIKCEQNLGH | NO: 38) |
| | | | | | | | | YGLSWYQQHDG | | DTMYWYKQDSKK | |
| | | | | | | | | GAPTFLSYNALD | | FLKIMFSYNNKE | |
| | | | | | | | | GLEETGRFSSFL | | LIINETVPNRFS | |
| | | | | | | | | SRSDSYGYLLLQ | | PKSPDKAHLNLH | |
| | | | | | | | | ELQMKDSASYF [symbol] | | INSLELGDSAVYF | |
| | | | | | | | | [symbol] | | [symbol] [symbol] | |
| | | | | | | | | GSGTRLLVR | | GPGTRLTVT | |
| | | | | | | | | (SEQ ID | | (SEQ | |
| | | | | | | | | NO: 13) | | ID NO: 20) | |
| P | TRAV14 | TRAJ30 | TRAC | TRBV6- | TRBD2 | TRBJ2- | TRBC2 | MSLSSLLKWTAS | CAMREGY | MSISLLCCAAFPL | CASSFSS |
| | DV4 | | | 6 | | 1 | | LWLGPGIAQKIT | RDDKIIF | LWAGPVNAGVT | GGAHEQF |
| | | | | | | | | QTQPGMFVQEK | (SEQ ID | QTPKFRILKIGQS | F |
| | | | | | | | | EAVTLDCTYDTS | NO: 103) | MTLQCTQDMNH | (SEQ ID |
| | | | | | | | | DQSYGLFWYKQP | | NYMYWYRQDPG | NO: 104) |
| | | | | | | | | SSGEMIFLIYQG | | MGLKLIYYSVGA | |
| | | | | | | | | SYDEQNATEGRY | | GITDKGEVPNGY | |
| | | | | | | | | SLNFQKARKSAN | | NVSRSTTEDFPL | |
| | | | | | | | | LVISASQLGDSA | | RLELAAPSQTSV | |
| | | | | | | | | MYF [symbol] | | YF [symbol] | |
| | | | | | | | | [symbol] FGKGTRLH | | [symbol] GPGTRLT | |
| | | | | | | | | ILP (SEQ ID | | VL (SEQ ID | |
| | | | | | | | | NO: 63) | | NO: 83) | |
| Q | TRAV12 | TRAJ28 | TRAC | TRBV9 | TRBD1 | TRBJ2- | TRBC2 | MISLRVLLVILW | CWNSGA | MGFRLLCCVAFC | CASSPLG |
| | -1 | | | | | 7 | | QLLSWVWSQRKE | GSYQLTF | LLGAGPVDSGVT | TGDYEQY |
| | | | | | | | | VEQDPGPFNVPE | (SEQ ID | QTPKHLITATGQ | F |
| | | | | | | | | GATVAFNCTYSN | NO: 105) | RVTLRCSPRSGD | (SEQ ID |
| | | | | | | | | SASQSFFWYRQ | | LSVYWYQQSLD | NO: 106) |
| | | | | | | | | DCRKEPKLLMSV | | QGLQFLIQYYNG | |
| | | | | | | | | YSSGNEDGRFTA | | EERAKGNILERFS | |
| | | | | | | | | QLNRASQYISLLI | | AQQFPDLHSELN | |
| | | | | | | | | RDSKLSDSATYL | | LSSLELGDSALYF | |
| | | | | | | | | [symbol] | | [symbol] | |
| | | | | | | | | [symbol] GKGTKLSVIP | | [symbol] GPGTRLTVT | |
| | | | | | | | | (SEQ ID | | (SEQ ID | |
| | | | | | | | | NO: 64) | | NO: 84) | |
| R | TRAV13 | TRAJ13 | TRAC | TRBV9 | None | TRBJ1- | TRBC1 | MAGIRALFMYLW | CAENSG | MGFRLLCCVAFC | CASSVGD |
| | -2 | | | | | 3 | | LQLDWVSRGESV | GYQKVTF | LLGAGPVDSGVT | HTIYF |
| | | | | | | | | GLHLPTLSVQEG | (SEQ ID | QTPKHLITATGQ | (SEQ ID |
| | | | | | | | | DNSIINCAYSNSA | NO: 107) | RVTLRCSPRSGD | NO: 108) |
| | | | | | | | | SDYFIWYKQESG | | LSVYWYQQSLD | |
| | | | | | | | | KGPQFIIDIRSNM | | QGLQFLIQYYNG | |
| | | | | | | | | DKRQGQRVTVLL | | EERAKGNILERFS | |
| | | | | | | | | NKTVKHLSLQIAA | | AQQFPDLHSELN | |
| | | | | | | | | TQPGDSAVYF [symbol] | | LSSLELGDSALYF | |
| | | | | | | | | [symbol] | | [symbol] | |
| | | | | | | | | GTGTKLQVIP | | GEGSWLTW | |
| | | | | | | | | (SEQ ID | | (SEQ ID | |
| | | | | | | | | NO: 65) | | NO: 85) | |
| S | TRAV25 | TRAJ47 | TRAC | TRBV27 | TRBD2 | TRBJ2- | TRBC1 | MLLITSMLVLWM | CAGPREY | MGPQLLGYVVLC | CASSYGG |
| | | | | | | 7 | | QLSQVNGQQVM | GNKLVF | LLGAGPLEAQVT | GSLVE |
| | | | | | | | | QIPQYQHVQEGE | (SEQ ID | QNPRYLITVTGKK | QYF |
| | | | | | | | | DFTTYCNSSTTL | NO: 109) | LTVTCSQNMNHE | (SEQ ID |
| | | | | | | | | SNIQWYKQRPG | | YMSWYRQDPGL | NO: 110) |
| | | | | | | | | GHPVFLIQLVKSG | | GLRQIYYSMNVE | |
| | | | | | | | | EVKKQKRLTFQF | | VTDKGDVPEGYK | |
| | | | | | | | | GEAKKNSSLHITA | | VSRKEKRNFPLIL | |
| | | | | | | | | TQTTDVGTYF [symbol] | | ESPSPNQTSLYF | |
| | | | | | | | | [symbol] G | | [symbol] | |
| | | | | | | | | AGTILRVKS | | [symbol] GPGTRLTVT | |
| | | | | | | | | (SEQ ID | | (SEQ ID | |
| | | | | | | | | NO: 66) | | NO: 86) | |
| T | TRAV1- | TRAJ31 | TRAC | TRBV2 | TRBD1 | TRBJ2- | TRBC2 | MWGAFLLYVSM | CAVRAQ | MDTWLVCWAIFS | CANAWGR |
| | 1 | | | | | 1 | | KMGGTAGQSLE | GNARL | LLKAGLTEPEVT | NEQFF |
| | | | | | | | | QPSEVTAVEGAI | MF | QTPSHQVTQMG | (SEQ ID |
| | | | | | | | | VQINCTYQTSGF | (SEQ ID | QEVILRCVPISNH | NO: 112) |
| | | | | | | | | YGLSWYQQHDG | NO: 111) | LYFYWYRQILGQ | |
| | | | | | | | | GAPTFLSYNALD | | KVEFLVSFYNNEI | |
| | | | | | | | | GLEETGRFSSFL | | SEKSEIFDDQFSV | |
| | | | | | | | | SRSDSYGYLLLQ | | ERPDGSNFTLKIR | |
| | | | | | | | | ELQMKDSASYF [symbol] | | STKLEDSAMYF [symbol] | |
| | | | | | | | | [symbol] | | [symbol] F | |
| | | | | | | | | [symbol] GDGTQLVVKP | | GPGTRLTVL | |
| | | | | | | | | (SEQ ID | | (SEQ ID | |
| | | | | | | | | NO: 67) | | NO: 87) | |
| U | TRAV26 | TRAJ39 | TRAC | TRBV15 | TRBD1 | TRBJ1- | TRBC1 | MKLVTSITVLLSL | CILPNA | MGPGLLHWMAL | CATRG |
| | -2 | | | | | 5 | | GIMGDAKTTQPN | GNMLTF | CLLGTGHGDAMV | TGTQPQ |
| | | | | | | | | SMESNEEEPVHL | (SEQ ID | IQNPRYQVTQFG | HF |
| | | | | | | | | PCNHSTISGTDYI | NO: 113) | KPVTLSCSQTLN | (SEQ ID |
| | | | | | | | | HWYRQLPSQGP | | HNVMYWYQQKS | NO: 114) |
| | | | | | | | | EYVIHGLTSNVN | | SQAPKLLFHYYD | |
| | | | | | | | | NRMASLAIAEDR | | KDFNNEADTPDN | |
| | | | | | | | | KSSTLILHRATLR | | FQSRRPNTSFCF | |
| | | | | | | | | DAAVYY [symbol] | | LDIRSPGLGDAA | |
| | | | | | | | | [symbol] GGGTRL | | MYL [symbol] | |
| | | | | | | | | MVKP (SEQ ID | | [symbol] GDGTRLSI | |
| | | | | | | | | NO: 68) | | L (SEQ ID | |
| | | | | | | | | | | NO: 88) | |
| V | TRAV38 | TRAJ57 | TRAC | TRBV3- | None | TRBJ2- | TRBC2 | MACPGFLWALVI | CAYRPY | MGCRLLCCWFC | CASSQGI |
| | -2DV8 | | | 1 | | 2 | | STCLEFSMAQTV | QGGSE | LLQAGPLDTAVS | LAAGE |
| | | | | | | | | TQSQPEMSVQE | KLVF | QTPKYLVTQMGN | LFF |
| | | | | | | | | AETVTLSCTYDT | (SEQ ID | DKSIKCEQNLGH | (SEQ ID |
| | | | | | | | | SESDYYLFWYKQ | NO: 115) | DTMYWYKQDSK | NO: 116) |
| | | | | | | | | PPSRQMILVIRQE | | KFLKIMFSYNNKE | |
| | | | | | | | | AYKQQNATENRF | | LIINETVPNRFSP | |
| | | | | | | | | SVNFQKAAKSFS | | KSPDKAHLNLHIN | |
| | | | | | | | | LKISDSQLGDAA | | SLELGDSAVYF [symbol] | |
| | | | | | | | | MYF [symbol] | | [symbol] | |
| | | | | | | | | [symbol] GKGTK | | [symbol] GEGSRLTVL | |
| | | | | | | | | LTVNP (SEQ ID | | (SEQ ID | |
| | | | | | | | | NO: 69) | | NO: 89) | |
| W | TRAV25 | TRAJ10 | TRAC | TRBV2 | TRBD1 | TRBJ2- | TRBC2 | MLLITSMLVLWM | CAGPRWL | MDTWLVCWAIFS | CASSVG |
| | | | | | | 7 | | QLSQVNGQQVM | TGGGNK | LLKAGLTEPEVT | GQGEV |
| | | | | | | | | QIPQYQHVQEGE | LTF | QTPSHQVTQMG | VQYF |
| | | | | | | | | DFTTYCNSSTTL | (SEQ ID | QEVILRCVPISNH | (SEQ ID |
| | | | | | | | | SNIQWYKQRPG | NO: 117) | LYFYWYRQILGQ | NO: 118) |
| | | | | | | | | GHPVFLIQLVKSG | | KVEFLVSFYNNEI | |
| | | | | | | | | EVKKQKRLTFQF | | SEKSEIFDDQFSV | |
| | | | | | | | | GEAKKNSSLHITA | | ERPDGSNFTLKIR | |
| | | | | | | | | TQTTDVGTYF [symbol] | | STKLEDSAMYF [symbol] | |
| | | | | | | | | [symbol] | | [symbol] | |
| | | | | | | | | [symbol] GTGTQLKVE | | [symbol] GPGTRLTVT | |
| | | | | | | | | L (SEQ ID | | (SEQ ID | |
| | | | | | | | | NO: 70) | | NO: 90) | |
| X | TRAV13 | TRAJ40 | TRAC | TRBV4- | TRBD1 | TRBJ2- | TRBC2 | MTSIRAVFIFLWL | CAAPP | MGCRLLCCAVLC | CASGEGD |
| | -1 | | | 1 | | 3 | | QLDLVNGENVEQ | PGYKYIF | LLGAVPIDTEVTQ | FAYTQY |
| | | | | | | | | HPSTLSVQEGDS | (SEQ ID | TPKHLVMGMTNK | (SEQ ID |
| | | | | | | | | AVIKCTYSDSASN | NO: 119) | KSLKCEQHMGH | NO: 120) |
| | | | | | | | | YFPWYKQELGK | | RAMYWVKQKAK | |
| | | | | | | | | GPQLIIDIRSNVG | | KPPELMFVYSYE | |
| | | | | | | | | EKKDQRIAVTLNK | | KLSINESVPSRFS | |
| | | | | | | | | TAKHFSLHITETQ | | PECPNSSLLNLH | |
| | | | | | | | | PEDSAVYF [symbol] | | LHALQPEDSALY | |
| | | | | | | | | [symbol] GTGT | | L [symbol] | |
| | | | | | | | | RLKVLA | | [symbol] GPGTRLTVL | |
| | | | | | | | | (SEQ ID | | (SEQ ID | |
| | | | | | | | | NO: 71) | | NO: 91) | |
| Y | TRAV30 | TRAJ26 | TRAC | TRBV11 | TRBD2 | TRBJ1- | TRBC1 | METLLKVLSGTLL | CGTELE | MGTRLLCWAALC | CASSLSG |
| | | | | -2 | | 3 | | WQLTWVRSQQP | YNGQN | LLGAELTEAGVA | GSGNT |
| | | | | | | | | VQSPQAVILREG | VFF | QSPRYKIIEKRQS | IYF |
| | | | | | | | | EDAVINCSSSKA | (SEQ ID | VAFWCNPISGHA | (SEQ ID |
| | | | | | | | | LYSVHWYRQKHG | NO: 121) | TLYWYQQILGQG | NO: 122) |
| | | | | | | | | EAPVFLMILLKG | | PKLLIQFQNNGV | |
| | | | | | | | | GEQKGHEKISAS | | VDDSQLPKDRFS | |
| | | | | | | | | FNEKKQQSSLYL | | AERLKGVDSTLKI | |
| | | | | | | | | TASQLSYSGTYF [symbol] | | QPAKLEDSAVYL | |
| | | | | | | | | [symbol] | | [symbol] | |
| | | | | | | | | [symbol] GPGTRLSVLP | | [symbol] GEGSWLTVV | |
| | | | | | | | | (SEQ ID | | (SEQ ID | |
| | | | | | | | | NO: 72) | | NO: 92) | |
| Z | TRAV5 | TRAJ41 | TRAC | TRBV29 | TRBD1 | TRBJ1- | TRBC1 | MKTFAGFSFLFL | CAESSR | MLSLLLLLLGLGS | CSVEDV |
| | | | | -1 | | 2 | | WLQLDCMSRGE | NSGYAL | VFSAVISQKPSR | PGGWG |
| | | | | | | | | DVEQSLFLSVRE | NF | DICQRGTSLTIQC | YTF |
| | | | | | | | | GDSSVINCTYTD | (SEQ ID | QVDSQVTMMFW | (SEQ ID |
| | | | | | | | | SSSTYLYWYKQE | NO: 123) | YRQQPGQSLTLI | NO: 124) |
| | | | | | | | | PGAGLQLLTYIF | | ATANQGSEATYE | |
| | | | | | | | | SNMDMKQDQRLT | | SGFVIDKFPISRP | |
| | | | | | | | | VLLNKKDKHLSL | | NLTFSTLTVSNM | |
| | | | | | | | | RIADTQTGDSAIY | | SPEDSSIYL [symbol] | |
| | | | | | | | | F [symbol] | | [symbol] G | |
| | | | | | | | | [symbol] GKGTSLLVT | | SGTRLTW | |
| | | | | | | | | P | | (SEQ ID | |
| | | | | | | | | (SEQ ID | | NO: 93) | |
| | | | | | | | | NO: 73) | | | |
| AA | TRAV38 | TRAJ21 | TRAC | TRBV11 | TRBD2 | TRBJ2 | TRBC2 | MACPGFLWALVI | CAYYVP | MGTRLLCWAALC | CASSTT |
| | -2DV8 | | | -2 | | -5 | | STCLEFSMAQTV | FNKFYF | LLGAELTEAGVA | SGGGQE |
| | | | | | | | | TQSQPEMSVQE | (SEQ ID | QSPRYKIIEKRQS | TQYF |
| | | | | | | | | AETVTLSCTYDT | NO: 125) | VAFWCNPISGHA | (SEQ ID |
| | | | | | | | | SESDYYLFWYKQ | | TLYWYQQILGQG | NO: 126) |
| | | | | | | | | PPSRQMILVIRQE | | PKLLIQFQNNGV | |
| | | | | | | | | AYKQQNATENRF | | VDDSQLPKDRFS | |
| | | | | | | | | SVNFQKAAKSFS | | AERLKGVDSTLKI | |
| | | | | | | | | LKISDSQLGDAA | | QPAKLEDSAVYL | |
| | | | | | | | | MYF [symbol] | | [symbol] | |
| | | | | | | | | [symbol] GSGTKLNVK | | [symbol] GPGTRLL | |
| | | | | | | | | P | | VL | |
| | | | | | | | | (SEQ ID | | (SEQ ID | |
| | | | | | | | | NO: 74) | | NO: 94) | |
| AB | TRAV12 | TRAJ31 | TRAC | TRBV7 | TRBD1 | TRBJ2 | TRBC2 | MKSLRVLLVILWL | CAVTSG | MGTRLLCWWLG | CASSLA |
| | -2 | | | -8 | | -7 | | QLSWVWSQQKE | RLMF | FLGTDHT GAGVS | AGEQYF |
| | | | | | | | | VEQNSGPLSVPE | (SEQ ID | QSPRYKVAKRG | (SEQ ID |
| | | | | | | | | GAIASLNCTYSD | NO: 127) | QDVALRCDPISG | NO: 128) |
| | | | | | | | | RGSQSFFWYRQY | | HVSLFWYQQALG | |
| | | | | | | | | SGKSPELIMFIY | | QGPEFLTYFQNE | |
| | | | | | | | | SNGDKEDGRFTA | | AQLDKSGLPSDR | |
| | | | | | | | | QLNKASQYVSLLI | | FFAERPEGSVST | |
| | | | | | | | | RDSQPSDSATYL | | LKIQRTQQEDSA | |
| | | | | | | | | [symbol] G | | VYL [symbol] | |
| | | | | | | | | DGTQLWKP | | [symbol] GPGTRLTVT | |
| | | | | | | | | (SEQ ID | | (SEQ ID | |
| | | | | | | | | NO: 75) | | NO: 95) | |
| AC | TRAV12 | TRAJ6 | TRAC | TRBV7 | None | TRBJ2- | TRBC2 | MISLRVLLVILWL | CVVNKRG | MGTRLLCWWLG | CASSAL |
| | -1 | | | -8 | | 7 | | QLSWVWSQRKE | SYIPTF | FLGTDHTGAGVS | GEQYF |
| | | | | | | | | VEQDPGPFNVPE | (SEQ ID | QSPRYKVAKRG | (SEQ ID |
| | | | | | | | | GATVAFNCTYSN | NO: 129) | QDVALRCDPISG | NO: 130) |
| | | | | | | | | SASQSFFWYRQ | | HVSLFWYQQALG | |
| | | | | | | | | DCRKEPKLLMSV | | QGPEFLTYFQNE | |
| | | | | | | | | YSSGNEDGRFTA | | AQLDKSGLPSDR | |
| | | | | | | | | QLNRASQYISLLI | | FFAERPEGSVST | |
| | | | | | | | | RDSKLSDSATYL | | LKIQRTQQEDSA | |
| | | | | | | | | [symbol] | | VYL [symbol] | |
| | | | | | | | | [symbol] GRGTSLIVHP | | [symbol] GPGTRLTVT | |
| | | | | | | | | (SEQ ID | | (SEQ ID | |
| | | | | | | | | NO: 76) | | NO: 96) | |
| AD | TRAV26 | TRAJ47 | TRAC | TRBV11 | TRBD1 | TRBJ1- | TRBC1 | MRLVARVTVFLT | CIVRGM | MGTRLLCWAALC | CASSLGP |
| | -1 | | | -2 | | 1 | | FGTIIDAKTTQPP | EYGNKL | LLGAELTEAGVA | GGSEAFF |
| | | | | | | | | SMDCAEGRAANL | VF | QSPRYKIIEKRQS | (SEQ ID |
| | | | | | | | | PCNHSTISGNEY | (SEQ ID | VAFWCNPISGHA | NO: 132) |
| | | | | | | | | VYWYRQIHSQGP | NO: 131) | TLYWYQQILGQG | |
| | | | | | | | | QYIIHGLKNNETN | | PKLLIQFQNNGV | |
| | | | | | | | | EMASLIITEDRKS | | VDDSQLPKDRFS | |
| | | | | | | | | STLILPHATLRDT | | AERLKGVDSTLKI | |
| | | | | | | | | AVYY [symbol] | | QPAKLEDSAVYL | |
| | | | | | | | | [symbol] GAGTIL | | [symbol] | |
| | | | | | | | | RVKS | | [symbol] GQGTRLTW | |
| | | | | | | | | (SEQ ID | | (SEQ ID | |
| | | | | | | | | NO: 77) | | NO: 97) | |
| AE | TRAV19 | TRAJ53 | TRAC | TRBV20 | None | TRBJ2- | TRBC2 | MLTASLLRAVIASI | CALSGSG | MLLLLLLLGPGSG | CSARSY |
| | | | | -1 | | 7 | | CVVSSMAQKVTQ | GSNYK | LGAVVSQHPSW | EQYF |
| | | | | | | | | AQTEISWEKEDV | LTF | VICKSGTSVKIEC | (SEQ ID |
| | | | | | | | | TLDCVYETRDTT | (SEQ ID | RSLDFQATTMFW | NO: 134) |
| | | | | | | | | YYLFWYKQPPSG | NO: 133) | YRQFPKQSLMLM | |
| | | | | | | | | ELVFLIRRNSFDE | | ATSNEGSKATYE | |
| | | | | | | | | QNEISGRYSWNF | | QGVEKDKFLINH | |
| | | | | | | | | QKSTSSFNFTITA | | ASLTLSTLTVTSA | |
| | | | | | | | | SQWDSAVYF [symbol] | | HPEDSSFYI [symbol] | |
| | | | | | | | | [symbol] | | [symbol] GPGTR | |
| | | | | | | | | [symbol] GKGTLLTVNP | | LTVT | |
| | | | | | | | | (SEQ ID | | (SEQ ID | |
| | | | | | | | | NO: 78) | | NO: 98) | |
| AF | TRAV1- | TRAJ13 | TRAC | TRBV29 | TRBD1 | TRBJ1- | TRBC1 | MWGAFLLYVSM | CAVTGG | MLSLLLLLLGLGS | CSVHRGV |
| | 1 | | | -1 | | 1 | | KMGGTAGQSLE | YQKVTF | VFSAVISQKPSR | NTEAFF |
| | | | | | | | | QPSEVTAVEGAI | (SEQ ID | DICQRGTSLTIQC | (SEQ ID |
| | | | | | | | | VQINCTYQTSGF | NO: 135) | QVDSQVTMMFW | NO: 136) |
| | | | | | | | | YGLSWYQQHDG | | YRQQPGQSLTLI | |
| | | | | | | | | GAPTFLSYNALD | | ATANQGSEATYE | |
| | | | | | | | | GLEETGRFSSFL | | SGFVIDKFPISRP | |
| | | | | | | | | SRSDSYGYLLLQ | | NLTFSTLTVSNM | |
| | | | | | | | | ELQMKDSASYF [symbol] | | SPEDSSIYL [symbol] | |
| | | | | | | | | [symbol] | | [symbol] GQ | |
| | | | | | | | | GTGTKLQVIP | | GTRLTW | |
| | | | | | | | | (SEQ ID | | (SEQ ID | |
| | | | | | | | | NO: 79) | | NO: 99) | |
| AG | TRAV29 | TRAJ43 | TRAC | TRBV7- | TRBD1 | TRBJ2- | TRBC2 | MAMLLGASVLIL | CAASA | MGTRLLCWWLG | CASSLG |
| | DV5 | | | 8 | | 7 | | WLQPDWVNSQQ | GNDMRF | FLGTDHT GAGVS | GYEQYF |
| | | | | | | | | KNDDQQVKQNS | (SEQ ID | QSPRYKVAKRG | (SEQ ID |
| | | | | | | | | PSLSVQEGRISIL | NO: 137) | QDVALRCDPISG | NO: 138) |
| | | | | | | | | NCDYTNSMFDYFL | | HVSLFWYQQALG | |
| | | | | | | | | WYKKYPAEGPTFL | | QGPEFLTYFQNE | |
| | | | | | | | | ISISSIKDKNED | | AQLDKSGLPSDR | |
| | | | | | | | | GRFTVFLNKSAK | | FFAERPEGSVST | |
| | | | | | | | | HLSLHIVPSQPG | | LKIQRTQQEDSA | |
| | | | | | | | | DSAVYF [symbol] | | VYL [symbol] | |
| | | | | | | | | [symbol] GAGTRLT | | [symbol] GPGTRLTVT | |
| | | | | | | | | VKP | | (SEQ ID | |
| | | | | | | | | (SEQ ID | | NO: 100) | |
| | | | | | | | | NO: 80) | | | |
| AH | TRAV12 | TRAJ43 | TRAC | TRBV28 | TRBD2 | TRBJ1- | TRBC1 | MISLRVLLVILWL | CWTYN | MGIRLLCRVAFC | CASSLL |
| | -1 | | | | | 2 | | QLSWVWSQRKE | DMRF | FLAVGLVDVKVT | SGSGYTF |
| | | | | | | | | VEQDPGPFNVPE | (SEQ ID | QSSRYLVKRTGE | (SEQ ID |
| | | | | | | | | GATVAFNCTYSN | NO: 139) | KVFLECVQDMDH | NO: 140) |
| | | | | | | | | SASQSFFWYRQ | | ENMFWYRQDPGL | |
| | | | | | | | | DCRKEPKLLMSV | | GLRLIYFSYDVK | |
| | | | | | | | | YSSGNEDGRFTA | | MKEKGDIPEGYS | |
| | | | | | | | | QLNRASQYISLLI | | VSREKKERFSLIL | |
| | | | | | | | | RDSKLSDSATYL | | ESASTNQTSMYL | |
| | | | | | | | | [symbol] GA | | [symbol] | |
| | | | | | | | | GTRLTVKP | | [symbol] GSGTRLTW | |
| | | | | | | | | (SEQ ID | | (SEQ ID | |
| | | | | | | | | NO: 81) | | NO: 101) | |
| AI | TRAV19 | TRAJ20 | TRAC | TRBV6- | TRBD1 | TRBJ2- | TRBC2 | MLTASLLRAVIASI | CALIPS | MSISLLCCAAFPL | CASSYS |
| | | | | 6 | | 7 | | CVVSSMAQKVTQ | NDYKLSF | LWAGPVNAGVTQ | MGEWS |
| | | | | | | | | AQTEISWEKEDV | (SEQ ID | TPKFRILKIGQS | YEQYF |
| | | | | | | | | TLDCVYETRDTT | NO: 141) | MTLQCTQDMNH | (SEQ ID |
| | | | | | | | | YYLFWYKQPPSG | | NYMYWYRQDPG | NO: 142) |
| | | | | | | | | ELVFLIRRNSFDE | | MGLKLIYYSVGA | |
| | | | | | | | | QNEISGRYSWNF | | GITDKGEVPNGY | |
| | | | | | | | | QKSTSSFNFTITA | | NVSRSTTEDFPL | |
| | | | | | | | | SQWDSAVYF [symbol] | | RLELAAPSQTSV | |
| | | | | | | | | [symbol] | | YF [symbol] | |
| | | | | | | | | GAGTTVTVRA | | [symbol] GPGT | |
| | | | | | | | | (SEQ ID | | RLTVT | |
| | | | | | | | | NO: 82) | | (SEQ ID | |
| | | | | | | | | | | NO: 102) | |

¹T cells were sequenced at single cell level using 10× Genomics single cell resolution paired immune TCR profiling. Sequencing reads were tagged with Chromium cellular barcodes and unique molecular identifiers and frequencies of complete T cell receptor sequences determined (see FIGS. 8-9). Alpha/beta chain pairs common at least two antigen-binding T cells and at least two antigen-activated T cells are reported.
²CDR3 regions are underlined and in bold.

All publications and patent applications mentioned in this specification are herein incorporated by reference to the same extent as if each individual publication or patent application was specifically and individually indicated to be incorporated by reference.

While preferred embodiments of the present invention have been shown and described herein, it will be obvious to those skilled in the art that such embodiments are provided by way of example only. It is intended that the following claims define the scope of the invention and that methods and structures within the scope of these claims and their equivalents be covered thereby.

What is claimed is:

1. A method for selection of T cell receptor clonotypes, comprising:
i) analyzing a mixture of T cells to identify antigen-binding T cells and antigen-activated T cells for a predetermined type of antigen; and
ii) identifying at least a portion of at least one T cell receptor sequence shared by at least one of the antigen-binding T cells and at least one of the antigen-activated T cells.

2. A method for selection of shared receptor sequences in lymphocytes, comprising:
i) analyzing a mixture of lymphocytes to identify stimulated lymphocytes and costimulated lymphocytes for a predetermined type of antigen; and
ii) identifying at least a portion of at least one receptor sequence shared by at least one of the stimulated lymphocytes and at least one of the costimulated lymphocytes.

3. A method for selection of T cell receptor clonotypes, comprising:
i) analyzing a mixture of naïve T cells to identify antigen-binding T cells and functional T cells for a predetermined type of antigen; and
ii) identifying at least a portion of at least one T cell receptor sequence shared by at least one of the antigen-binding T cells and at least one of the functional T cells.

4. A method for selection of T cell receptors, comprising:
i) binding at least a first antigen-binding T cell to at least a first one of a predetermined type of antigen, comprising: contacting a first plurality of T cells with the first one of the predetermined type of antigen;
ii) activating at least a first functional T cell, comprising: contacting a second plurality of T cells with a plurality of cells that present at least a second one of the predetermined type of antigen; and
iii) identifying at least a portion of at least one T cell receptor sequence that is common to the at least one antigen-binding T cell and the at least one functional T cell.

5. A method for selection of T cell receptors, comprising:
i) binding at least a first antigen-binding T cell present in a first plurality of T cells to at least a first one of a Class I P-MHC protein multimer, which P is a predetermined type of antigen, comprising: contacting the first plurality of T cells with the first one of the Class I P-MHC protein multimer;
ii) activating at least a first functional T cell present in a second plurality of T cells, comprising: contacting the second plurality of T cells with a plurality of cells that present at least a first one of a Class II P-MHC protein multimer; and
iii) identifying at least a portion of at least one T cell receptor sequence that is common to the at least one antigen-binding T cell and the at least one functional T cell.

6. A method for selection of T cell receptors, comprising:
i) binding at least a first antigen-binding T cell present in a first plurality of T cells to at least a first one of a Class I P-MHC protein multimer, where P is a predetermined type of antigen, comprising: contacting the first plurality of T cells with the first one of the Class I P-MHC protein multimer;
ii) activating at least a first functional T cell present in a second plurality of T cells, comprising: contacting the second plurality of T cells with a plurality of cells that present at least a first Class I P-MHC protein; and
iii) identifying at least a portion of at least one T cell receptor sequence that is common to the at least one antigen-binding T cell and the at least one functional T cell.

7. A method for selection of T cell receptors, comprising:
i) isolating a first T cell from a plurality of T cells, the first T cell bound to a P-loaded MHC protein, which P is a predetermined type of antigen;
ii) further isolating a second T cell from the plurality of T cells, the second T cell expressing at least one biomarker indicative of activation by the predetermined type of antigen; and
iii) matching at least a portion of a T cell receptor sequence of the first T cell with at least a portion of a T cell receptor sequence of the second T cell.

8. A method for detecting functional T cell receptor clonotypes, comprising:
i) isolating, from a population of PBMCs, at least one T cell that binds to a predetermined type of antigen;
ii) forming a plurality of cognate T cells, comprising: expanding the isolated at least one T cell;
iii) activating at least a first functional T cell, comprising: contacting T cells derived from the plurality of cognate T cells with at least one of a plurality of activation agents that is immunogenic for the predetermined type of antigen; and
iv) confirming that the at least a first functional T cell is configured to bind to a P-loaded MHC protein, which P is the predetermined type of antigen.

9. A method for detecting antigen-binding T cells, comprising:
i) isolating, from a population of PBMCs, at least one T cell that binds to a predetermined type of antigen;
ii) forming a plurality of cognate T cells, comprising: expanding the isolated at least one T cell;
iii) binding at least a first binding T cell to at least a first binding agent, comprising: contacting T cells derived from the plurality of cognate T cells with at least one of a plurality of binding agents, the at least one of the plurality of binding agents comprising the predetermined type of antigen; and
iv) confirming that the at least a first binding T cell is configured to be activated by a cell that presents the predetermined type of antigen.

10. A method for selection of T cell receptors specific for a predetermined type of antigen, comprising:
i) isolating a first plurality of T cells, at least a portion of the first plurality of T cells bound to a plurality of P-loaded MHC proteins, which P is the predetermined type of antigen;
ii) further isolating a second plurality of T cells, at least a portion of the second plurality of T cells upregulating one or more activation signaling molecules and/or expressing one or more activation markers in the presence of a plurality of activation agents, wherein at least one of the plurality of activation agents is immunogenic for the predetermined type of antigen; and
iii) identifying at least a portion of at least one T cell receptor sequence that is common to both the at least a portion of the first plurality of T cells and the at least a portion of the second plurality of T cells.

11. A method for selection of T cell receptors specific for a predetermined type of antigen, comprising:
i) isolating a first plurality of T cells, at least a portion of the first plurality of T cells expressing one or more first activation markers in the presence of a plurality of first activation agents;
ii) further isolating a second plurality of T cells, at least a portion of the second plurality of T cells upregulating one or more second activation markers and/or expressing one or more activation signaling molecules in the presence of a plurality of second activation agents; and
iii) identifying at least one of the portion of the first plurality of T cells and at least one of the portion of the second T cells having—
a) at least a portion of at least one T cell receptor sequence in common; and
b) dissociation constants with a P-loaded MHC protein that are below a threshold value, which P is the predetermined type of antigen.

12. A method for negative selection of T cell receptor clonotypes, comprising:
i) analyzing a mixture of T cells to identify first antigen-binding T cells and first antigen-activated T cells for a predetermined first type of antigen and second antigen-activated T cells for a predetermined second type of antigen; and
ii) identifying at least a portion of at least one T cell receptor sequence—
a) shared by at least one of the first antigen-binding T cells and at least one of the first antigen-activated T cells; and
b) not shared with any of the second antigen-activated T cells.

13. A method for negative selection of T cell receptor clonotypes, comprising:
i) analyzing a mixture of T cells to identify first antigen-activated T cells and first antigen-binding T cells for a predetermined first type of antigen and second antigen-binding T cells for a predetermined second type of antigen; and
ii) identifying at least a portion of at least one T cell receptor sequence—
a) shared by at least one of the first antigen-binding T cells and at least one of the first antigen-activated T cells; and
b) not shared with any of the second antigen-binding T cells.

14. A method for identifying a T cell activation marker, comprising:
i) contacting a first plurality of T cells with a plurality of P-presenting cells, the first plurality of T cells comprising a plurality of P-binding T cells, which P is a predetermined type of antigen;
ii) measuring a plurality of expression rate profiles for at least a portion of the contacted plurality of P-binding T cells;
iii) partitioning, into a plurality of T cell clusters, the at least a portion of the contacted plurality of P-binding T cells;
iv) measuring a functional response to P in at least two T cells present in the at least a portion of the contacted plurality of P-binding T cells;
v) mapping the expression rate profiles to the plurality of T cell clusters to identify one of the plurality of T cell clusters comprising the at least two T cells; and
vi) identifying an activation marker that is expressed by the at least two T cells.

15. A method for screening a candidate antigen for an antigen-specific vaccine, comprising:
i) isolating, from a population of PBMCs, at least one T cell that binds to the candidate antigen;
ii) forming a plurality of cognate T cells, comprising: expanding the isolated at least one T cell; and
iii) activating at least a first functional T cell, comprising: contacting T cells derived from the plurality of cognate T cells with at least one of a plurality of activation agents that is immunogenic for the candidate antigen.

16. A method for screening a candidate neoantigen for immunogenicity, comprising:
i) isolating, from a population of PBMCs, at least one T cell that binds to the candidate neoantigen;
ii) forming a plurality of cognate T cells, comprising: expanding the isolated at least one T cell; and
iii) activating at least a first functional T cell, comprising: contacting T cells derived from the plurality of cognate T cells with at least one of a plurality of activation agents that is immunogenic for the candidate neoantigen.

17. The method of any one of claims 1 to 16, wherein the analyzing comprises analyzing a first portion of the mixture to identify the antigen-binding T cells and separately analyzing a second portion of the mixture to identify the antigen-activated T cells.

18. The method of any one of claims 1 to 17, wherein the analyzing the first portion of the mixture comprises detecting one or more T cells bound to a P-loaded MHC protein, wherein P is the predetermined type of antigen.

19. The method of any one of claims 1 to 18, wherein the P-loaded MHC protein is coupled to a magnetic bead.

20. The method of any one of claims 1 to 19, wherein the detecting the one or more T cells bound to a P-loaded MHC protein comprises isolating the one or more T cells bound to the P-loaded MHC protein via magnetic separation.

21. The method of any one of claims 1 to 20, wherein the P-loaded MHC protein is coupled to a fluorophore.

22. The method of any one of claims 1 to 21, wherein the one or more T cells bound to the P-loaded MHC protein are detected and isolated via fluorescence flow cytometry.

23. The method of any one of claims 1 to 22, wherein the detecting the one or more T cells bound to a P-loaded MHC protein comprises passing the one or more T cells bound to the P-loaded MHC protein through a fluorescence flow cytometry device.

24. The method of any one of claims 1 to 23, wherein the MHC protein is an MHC Class I protein.

25. The method of any one of claims 1 to 24, wherein the P-loaded MHC protein is present in a P-loaded MHC protein multimer.

26. The method of any one of claims 1 to 25, wherein the separately analyzing a second portion of the mixture to identify the antigen-activated T cells comprises detecting one or more T cells expressing one or more activation markers.

27. The method of any one of claims 1 to 26, wherein the detecting the one or more T cells expressing one or more activation markers comprises isolating the one or more T cells expressing the one or more activation markers via magnetic separation.

28. The method of any one of claims 1 to 27, wherein the detecting the one or more T cells expressing one or more activation markers comprises passing the one or more T cells expressing the one or more activation markers through a fluorescence flow cytometry device.

29. The method of any one of claims 1 to 28, wherein the method is exclusive of in vitro priming.

30. The method of any one of claims 1 to 29, wherein the predetermined type of antigen is a peptide.

31. The method of any one of claims 1 to 30, wherein the peptide consists of 8-15 amino acids.

32. The method of any one of claims 1 to 31, wherein the peptide consists of 12-40 amino acids.

33. The method of any one of claims 1 to 32, wherein the predetermined type of antigen is derived from a tumor (for example a solid tumor).

34. The method of any one of claims 1 to 33, wherein the predetermined type of antigen is presented on a tumor.

35. The method of any one of claims 1 to 34, wherein the predetermined type of antigen is a neoantigen derived from a tumor.

36. The method of any one of claims 1 to 35, wherein the predetermined type of antigen is a personalized antigen.

37. The method of any one of claims 1 to 36, wherein the predetermined type of antigen is a shared tumor antigen.

38. The method of any one of claims 1 to 37, wherein the shared tumor antigen is a cancer/testis antigen.

39. The method of any one of claims 1 to 38, wherein the shared tumor antigen is a cancer/testis-like antigen.

40. The method of any one of claims 1 to 39, wherein the shared tumor antigen is a tumor associated peptide antigen.

41. The method of any one of claims 1 to 40, wherein the predetermined type of antigen is characteristic of a particular type of tumor.

42. The method of any one of claims 1 to 41, wherein the predetermined type of antigen is a tumor associated peptide antigen.

43. The method of any one of claims 1 to 42, wherein the at least a portion of at least one T cell receptor sequence comprises at least one T cell receptor clonotype.

44. The method of any one of claims 1 to 43, wherein the at least a portion of at least one T cell receptor sequence comprises at least one T cell receptor alpha chain, at least one T cell receptor beta chain, or at least one pair of T cell receptor alpha and beta chains.

45. The method of any one of claims 1 to 44, wherein the identifying comprises: sequencing the at least one binding T cell at a single cell level.

46. The method of any one of claims 1 to 45, wherein the identifying comprises: sequencing the at least one functional T cell at a single cell level.

47. The method of any one of claims 1 to 46, wherein the at least a portion of at least one T cell receptor sequence comprises at least one CDR3 sequence.

48. The method of any one of claims 1 to 47, wherein the at least one of the antigen-binding T cells and the at least one of the antigen-activated T cells are together less than 1000 T cells per 1,000,000 T cells present in the mixture of T cells.

49. The method of any one of claims 1 to 48, wherein the method further comprises: preparing the mixture of T cells, comprising:
i) isolating, from a population of PBMCs, at least one T cell that binds to the predetermined type of antigen; and
ii) expanding the isolated at least one T cell.

50. The method of any one of claims 1 to 49, wherein the at least one T cell is at least two T cells, wherein the expanding comprises polyclonally expanding the at least two T cells.

51. The method of any one of claims 1 to 50, wherein the at least one of the antigen-binding T cells and the at least one of the antigen-activated T cells are together less than 1000 T cells per 10,000,000 T cells present in the population of PBMCs.

52. The method of any one of claims 1 to 51, wherein the mixture of stimulated lymphocytes and costimulated lymphocytes is T cells.

53. The method of any one of claims 1 to 52, wherein the mixture of stimulated lymphocytes and costimulated lymphocytes is B cells.

54. The method of any one of claims 1 to 53, wherein the mixture of stimulated lymphocytes and costimulated lymphocytes is natural killer cells.

55. The method of any one of claims 1 to 54, wherein the analyzing comprises analyzing a first portion of the mixture to identify the stimulated lymphocytes and separately analyzing a second portion of the mixture to identify the costimulated lymphocytes.

56. The method of any one of claims 1 to 55, wherein the analyzing the first portion of the mixture comprises detecting one or more stimulated lymphocytes bound to a protein, wherein the protein comprises the predetermined type of antigen.

57. The method of any one of claims 1 to 56, wherein the protein is coupled to a magnetic bead.

58. The method of any one of claims 1 to 57, wherein the detecting the one or more stimulated lymphocytes bound to the protein comprises isolating the one or more stimulated lymphocytes bound to the protein via magnetic separation.

59. The method of any one of claims 1 to 58, wherein the protein is coupled to a fluorophore.

60. The method of any one of claims 1 to 59, wherein the one or more stimulated lymphocytes bound to the protein is detected and isolated via fluorescence flow cytometry.

61. The method of any one of claims 1 to 60, wherein the detecting the one or more stimulated lymphocytes bound to the protein comprises passing the one or more stimulated lymphocytes bound to the protein through a fluorescence flow cytometry device.

62. The method of any one of claims 1 to 61, wherein the separately analyzing a second portion of the mixture to identify the costimulated lymphocytes comprises detecting one or more stimulated lymphocytes expressing one or more markers.

63. The method of any one of claims 1 to 62, wherein the detecting the one or more stimulated lymphocytes expressing one or more markers comprises isolating the one or more stimulated lymphocytes expressing the one or more markers via magnetic separation.

64. The method of any one of claims 1 to 63, wherein the detecting the one or more stimulated lymphocytes expressing one or more markers comprises passing the one or more stimulated lymphocytes expressing the one or more markers through a fluorescence flow cytometry device.

65. The method of any one of claims 1 to 64, wherein the method is exclusive of priming with professional antigen presenting cells.

66. The method of any one of claims 1 to 65, wherein the predetermined type of antigen is a peptide.

67. The method of any one of claims 1 to 66, wherein the peptide consists of 8-15 amino acids.

68. The method of any one of claims 1 to 67, wherein the peptide consists of 12-40 amino acids.

69. The method of any one of claims 1 to 68, wherein the predetermined type of antigen is derived from a tumor.

70. The method of any one of claims 1 to 69, wherein the predetermined type of antigen is presented on a tumor.

71. The method of any one of claims 1 to 70, wherein the predetermined type of antigen is a neoantigen derived from a tumor.

72. The method of any one of claims 1 to 71, wherein the predetermined type of antigen is a personalized antigen.

73. The method of any one of claims 1 to 72, wherein the personalized antigen is a personalized neoantigen selected based on a model.

74. The method of any one of claims 1 to 73, wherein the predetermined type of antigen is a shared tumor antigen.

75. The method of any one of claims 1 to 74, wherein the shared tumor antigen is a cancer/testis antigen.

76. The method of any one of claims 1 to 75, wherein the shared tumor antigen is a cancer/testis-like antigen.

77. The method of any one of claims 1 to 76, wherein the shared tumor antigen is a tumor associated peptide antigen.

78. The method of any one of claims 1 to 77, wherein the predetermined type of antigen is characteristic of a particular type of tumor.

79. The method of any one of claims 1 to 78, wherein the predetermined type of antigen is a tumor associated peptide antigen.

80. The method of any one of claims 1 to 79, wherein the at least a portion of at least one receptor sequence comprises at least one receptor clonotype.

81. The method of any one of claims 1 to 80, wherein the at least a portion of at least one receptor sequence comprises at least one receptor alpha chain, at least one receptor beta chain, or at least one pair of receptor alpha and beta chains.

82. The method of any one of claims 1 to 81, wherein the identifying comprises: sequencing the at least one of the stimulated lymphocytes at a single cell level.

83. The method of any one of claims 1 to 82, wherein the identifying comprises: sequencing the at least one of the costimulated lymphocytes at a single cell level.

84. The method of any one of claims 1 to 83, wherein the at least a portion of at least one receptor sequence comprises at least one antigen recognition sequence.

85. The method of any one of claims 1 to 84, wherein the at least one of the stimulated lymphocytes and the at least one of the costimulated lymphocytes are together be less than 1000 T cells per 1,000,000 T cells present in the mixture of lymphocytes.

86. The method of any one of claims 1 to 85, wherein the method further comprises: preparing the mixture of lymphocytes, comprising:
i) isolating, from a population of PBMCs, at least one lymphocyte that binds to the predetermined type of antigen; and
ii) expanding the isolated at least one lymphocyte.

87. The method of any one of claims 1 to 86, wherein the at least two lymphocytes bind to the predetermined type of antigen, wherein the expanding comprises polyclonally expanding the at least two lymphocytes.

88. The method of any one of claims 1 to 87, wherein the at least one of the stimulated lymphocytes and the at least one of the costimulated lymphocytes together are less than 1000 T cells per 10,000,000 lymphocytes present in the population of PBMCs.

89. The method of any one of claims 1 to 88, wherein the mixture of lymphocytes is a product of priming with professional antigen presenting cells.

90. The method of any one of claims 1 to 89, wherein the plurality of cells that present at least the second one of the predetermined type of antigen present a plurality of the predetermined type of antigen within a predetermined concentration range.

91. The method of any one of claims 1 to 90, wherein the plurality of cells that present at least the second one of the predetermined type of antigen are prepared by pulsing the plurality of cells with a quantity of the predetermined type of antigen.

92. The method of any one of claims 1 to 91, wherein the predetermined concentration range is based on an expected concentration of the predetermined type of antigen in a tumor.

93. The method of any one of claims 1 to 92, wherein the binding comprises binding the at least a first binding T cell to a P-loaded MHC protein, wherein P is the predetermined type of antigen.

94. The method of any one of claims 1 to 93, wherein the MHC protein is an MHC Class I protein.

95. The method of any one of claims 1 to 94, wherein the P-loaded MHC protein is present in a P-loaded MHC protein multimer.

96. The method of any one of claims 1 to 95, wherein the first plurality of T cells and the second plurality of T cells are derived from a common population of PBMCs.

97. The method of any one of claims 1 to 96, wherein the first plurality of T cells and the second plurality of T cells are derived from one or more healthy donors.

98. The method of any one of claims 1 to 97, wherein the one or more healthy donors are at least partially human leukocyte antigen (HLA)-matched to a subject.

99. The method of any one of claims 1 to 98, wherein the one or more healthy donors are at least partially HLA-matched to a subject for presenting the predetermined type of antigen.

100. The method of any one of claims 1 to 99, wherein the one or more healthy donors are completely HLA-matched to a subject.

101. The method of any one of claims 1 to 100, wherein the one or more healthy donors are selectively HLA-matched to a subject.

102. The method of any one of claims 1 to 101, wherein the one or more healthy donors are matched to a subject for HLA-A.

103. The method of any one of claims 1 to 102, wherein the one or more healthy donors are matched to a subject for HLA-B.

104. The method of any one of claims 1 to 103, wherein the one or more healthy donors are matched to a subject for HLA-C.

105. The method of any one of claims 1 to 104, wherein the one or more healthy donors are matched to a subject for HLA-DP.

106. The method of any one of claims 1 to 105, wherein the one or more healthy donors are matched to a subject for HLA-DQ.

107. The method of any one of claims 1 to 106, wherein the one or more healthy donors are matched to a subject for HLA-DR.

108. The method of any one of claims 1 to 107, wherein the one or more healthy donors are at least partially HLA-mismatched to a subject.

109. The method of any one of claims 1 to 108, wherein the one or more healthy donors are completely HLA-mismatched to a subject.

110. The method of any one of claims 1 to 109, wherein the one or more healthy donors are selectively HLA-mismatched to a subject.

111. The method of any one of claims 1 to 110, wherein the one or more healthy donors are mismatched to a subject for HLA-A.

112. The method of any one of claims 1 to 111, wherein the one or more healthy donors are mismatched to a subject for HLA-B.

113. The method of any one of claims 1 to 112, wherein the one or more healthy donors are mismatched to a subject for HLA-C.

114. The method of any one of claims 1 to 113, wherein the one or more healthy donors are mismatched to a subject for HLA-DP.

115. The method of any one of claims 1 to 114, wherein the one or more healthy donors are mismatched to a subject for HLA-DQ.

116. The method of any one of claims 1 to 115, wherein the one or more healthy donors are mismatched to a subject for HLA-DR.

117. The method of any one of claims 1 to 116, wherein the one or more healthy donors are mismatched to a subject for HLA-A, HLA-B, HLA-C, HLA-DP, HLA-DQ, HLA-DR, or a combination of two or more of the foregoing.

118. The method of any one of claims 1 to 117, wherein the first plurality of T cells and the second plurality of T cells comprise naïve CD8⁺ T cells.

119. The method of any one of claims 1 to 118, wherein the first plurality of T cells and the second plurality of T cells comprise naïve T cells.

120. The method of any one of claims 1 to 119, wherein the first plurality of T cells and the second plurality of T cells comprise memory T cells.

121. The method of any one of claims 1 to 120, wherein the first plurality of T cells and the second plurality of T cells comprise CD8⁺ T cells.

122. The method of any one of claims 1 to 121, wherein the first plurality of T cells and the second plurality of T cells comprise CD4⁺ T cells.

123. The method of any one of claims 1 to 122, wherein the first plurality of T cells and the second plurality of T cells comprise CD4⁺ CD8⁺ T cells.

124. The method of any one of claims 1 to 123, wherein the first plurality of T cells and the second plurality of T cells comprise CD4⁻ CD8⁺ T cells.

125. The method of any one of claims 1 to 124, wherein the first plurality of T cells and the second plurality of T cells comprise CD4⁺ CD8⁻ T cells.

126. The method of any one of claims 1 to 125, wherein the plurality of cells that present at least the second one of the predetermined type of antigen comprise one or more tumor cells.

127. The method of any one of claims 1 to 126, wherein the plurality of cells that present at least the second one of the predetermined type of antigen comprise one or more dendritic cells.

128. The method of any one of claims 1 to 127, wherein the plurality of cells that present at least the second one of the predetermined type of antigen comprise one or more macrophages.

129. The method of any one of claims 1 to 128, wherein the plurality of cells that present at least the second one of the predetermined type of antigen comprise one or more monocytes.

130. The method of any one of claims 1 to 129, wherein the plurality of cells that present at least the second one of the predetermined type of antigen comprise one or more B cells.

131. The method of any one of claims 1 to 130, wherein the plurality of cells that present at least the second one of the predetermined type of antigen comprise one or more the plurality of cells that present at least the second one of the predetermined type of antigen express the predetermined type of antigen.

132. The method of any one of claims 1 to 131, wherein the method further comprises: detecting the binding via flow cytometry.

133. The method of any one of claims 1 to 132, wherein the first one of the predetermined type of antigen is coupled to a magnetic bead, where the method further comprises: detecting the at least a first antigen-binding T cell via magnetic separation.

134. The method of any one of claims 1 to 133, wherein the method further comprises: detecting the activating via flow cytometry.

135. The method of any one of claims 1 to 134, wherein the method further comprises: detecting the at least a first functional T cell via magnetic separation.

136. The method of any one of claims 1 to 135, wherein the method further comprises: detecting the activating, comprising: detecting one or more biomarkers.

137. The method of any one of claims 1 to 136, wherein the one or more biomarkers comprises CD137.

138. The method of any one of claims 1 to 137, wherein the method further comprises:
detecting the activating, comprising: detecting presence of one or more molecules indicative of T cell activation.

139. The method of any one of claims 1 to 138, wherein the one or more molecules comprises interferon gamma.

140. The method of any one of claims 1 to 139, wherein the method further comprises:
detecting the activating, comprising: detecting T cell proliferation.

141. The method of any one of claims 1 to 140, wherein the method further comprises:
deriving the plurality of T cells from at least two T cells that are separately bound to at least two P-loaded MHC proteins.

142. The method of any one of claims 1 to 141, wherein the deriving comprises expanding the at least a first T cell and the at least a second T cell.

143. The method of any one of claims 1 to 142, wherein the expanding comprises polyclonally expanding the at least a first T cell and the at least a second T cell.

144. The method of any one of claims 1 to 143, wherein the at least a first T cell and the at least a second T cell are in a mixture during the expanding.

145. The method of any one of claims 1 to 144, wherein the at least a first T cell and the at least a second T cell are separated from one another prior to the expanding.

146. The method of any one of claims 1 to 145, wherein the forming comprises indirect T cell receptor cross-linking.

147. The method of any one of claims 1 to 146, wherein the forming is limited to a single polyclonal expansion.

148. The method of any one of claims 1 to 147, wherein the forming comprises multiple polyclonal expansions.

149. The method of any one of claims 1 to 148, wherein at least one of the multiple polyclonal expansions is followed by isolating at least one further T cell that binds to the predetermined type of antigen.

150. The method of any one of claims 1 to 149, wherein the at least a first functional T cell has a dissociation constant with the P-loaded MHC protein of less than 50 μM.

151. The method of any one of claims 1 to 150, wherein the at least a first functional T cell has a half-life with the P-loaded MHC protein of between 2 second and 10 seconds.

152. The method of any one of claims 1 to 151, wherein the predetermined type of antigen is a tumor associated peptide antigen, wherein the at least a first functional T cell has: i) a dissociation constant with the P-loaded MHC protein of less than 50 μM; and ii) a half-life with the P-loaded MHC protein in the range of 2-10 seconds.

153. The method of any one of claims 1 to 152, wherein the least one of the plurality of activation agents is antigenic for the predetermined type of antigen.

154. The method of any one of claims 1 to 153, wherein the cell that presents the predetermined type of antigen is an antigen presenting cell.

155. The method of any one of claims 1 to 154, wherein the antigen presenting cell is a professional antigen presenting cell.

156. The method of any one of claims 1 to 155, wherein the at least a portion of the at least one T cell receptor sequence is present in at least 0.005% of the at least a portion of the first plurality of T cells and the at least a portion of the second plurality of T cells combined.

157. The method of any one of claims 1 to 156, wherein the at least one of the plurality of activation agents is antigenic for the predetermined type of antigen.

158. The method of any one of claims 1 to 157, wherein at least one of the plurality of first activation agents is immunogenic for the predetermined type of antigen, and/or at least one of the plurality of second activation agents is immunogenic for the predetermined type of antigen.

159. The method of any one of claims 1 to 158, wherein at least one of the plurality of first activation agents is antigenic for the predetermined type of antigen, and/or at least one of the plurality of second activation agents is antigenic for the predetermined type of antigen.

160. The method of any one of claims 1 to 159, wherein at least one of the plurality of first activation agents comprises the predetermined type of antigen, and/or at least one of the plurality of second activation agents comprises the predetermined type of antigen.

161. The method of any one of claims 1 to 160, wherein at least one of the plurality of first activation agents is a cell that presents the predetermined type of antigen, and/or at least one of the plurality of second activation agents is a cell that presents the predetermined type of antigen.

162. The method of any one of claims 1 to 161, wherein at least one of the plurality of first activation agents comprises P-loaded MHC protein, and/or at least one of the plurality of second activation agents P-loaded MHC protein.

163. The method of any one of claims 1 to 162, wherein at least one of the plurality of first activation agents is a cell that endogenously expresses the predetermined type of antigen, and/or at least one of the plurality of second activation agents is a cell that endogenously expresses the predetermined type of antigen.

164. The method of any one of claims 1 to 163, wherein at least one of the plurality of first activation agents comprises a P-loaded MHC protein, and/or at least one of the plurality of second activation agents is a cell that endogenously expresses the predetermined type of antigen.

165. The method of any one of claims 1 to 164, wherein the dissociation constants correspond to binding between the at least a portion of at least one T cell receptor sequence and the P-loaded MHC protein.

166. The method of any one of claims 1 to 165, wherein the threshold value is less than 1000 μM.

167. The method of any one of claims 1 to 166, wherein the predetermined first type of antigen is a first peptide and the predetermined second type of antigen is a second peptide.

168. The method of any one of claims 1 to 167, wherein the first peptide is expressed by a variant of a gene that expresses the second peptide.

169. The method of any one of claims 1 to 168, wherein the first peptide is expressed by an allele of a gene that expresses the second peptide.

170. The method of any one of claims 1 to 169, wherein the second peptide is expressed by a wild type gene.

171. The method of any one of claims 1 to 170, wherein the first peptide is a neoantigen and the second peptide is expressed by a related wild type gene.

172. The method of any one of claims 1 to 171, wherein the first peptide and the second peptide differ by at least 5 amino acids.

173. The method of any one of claims 1 to 172, wherein the first peptide and the second peptide differ by between 5 and 15 amino acids.

174. The method of any one of claims 1 to 173, wherein the first peptide and the second peptide have sequence identity of less than 75%.

175. The method of any one of claims 1 to 174, wherein the first peptide and the second peptide have sequence identity of between 55% and 80%.

176. The method of any one of claims 1 to 175, wherein identifying the first antigen-activated T cells comprises contacting a portion of the mixture of T cells with cells that endogenously present the predetermined first type of antigen.

177. The method of any one of claims 1 to 176, wherein identifying the second antigen-activated T cells comprises contacting a portion of the mixture of T cells with cells that endogenously present the predetermined second type of antigen.

178. The method of any one of claims 1 to 177, wherein identifying the first antigen-activated T cells comprises contacting a portion of the mixture of T cells with cells that have been loaded with the predetermined first type of antigen.

179. The method of any one of claims 1 to 178, wherein identifying the second antigen-activated T cells comprises contacting a portion of the mixture of T cells with cells that have been loaded with the predetermined second type of antigen.

180. The method of any one of claims 1 to 179, wherein the P-binding T cells are identified using a bioinformatics filter that compares at least portions of T cell receptor sequences of the at least a portion of the contacted plurality of P-binding T cells with at least portions of predetermined T cell receptor sequences.

181. The method of any one of claims 1 to 180, wherein the partitioning comprises: partitioning the contacted plurality of P-binding T cells into groups, at least one of the groups consisting of T cells having at least portions of T cell receptor sequences in common.

182. The method of any one of claims 1 to 181, wherein the partitioning comprises: partitioning the contacted plurality of P-binding T cells into groups, at least one of the groups consisting of T cells having at least portions of T cell receptor sequences characterized by sequence identities of at least 70% to one another.

183. The method of any one of claims 1 to 182, wherein the partitioning comprises: partitioning the contacted plurality of P-binding T cells into groups, at least one of the groups consisting of T cells having at least portions of T cell receptor sequences that differ by at most 1 amino acid between one another.

184. The method of any one of claims 1 to 183, wherein the partitioning comprises: partitioning the contacted plurality of P-binding T cells into groups, at least one of the groups consisting of T cells having at least portions of T cell receptor sequences that differ by only conservative substitutions.

185. The method of any one of claims 1 to 184, wherein the partitioning comprises grouping of lymphocyte interactions by paratope hotspots (GLIPH).

186. The method of any one of claims 1 to 185, wherein the at least portions of T cell receptor sequences in common are at least portions of a CDR3 region.

187. The method of any one of claims 1 to 186, wherein the at least portions of the CDR3 region comprises a linear amino acid sequences having lengths of between 6 and 35 amino acids.

188. The method of any one of claims 1 to 187, wherein the at least portions of the CDR3 region are exclusive of stem regions.

189. The method of any one of claims 1 to 188, wherein the at least portions of the CDR3 region comprise CDR3 beta chain portions.

190. The method of any one of claims 1 to 189, wherein the partitioning is performed using an algorithm.

191. The method of any one of claims 1 to 190, wherein the algorithm comprises a similarity analysis of the plurality of expression rate profiles.

192. The method of any one of claims 1 to 191, wherein the plurality of expression rate profiles comprise expression rates for one or more activation markers indicative of a functional response to P.

193. The method of any one of claims 1 to 192, wherein the one or more activation markers comprises CD137, CD69, CD25, Ki67, CD107, CD122, CD27, CD28, CD95, CD134, killer-cell lectin like receptor G1 (KLRG1), CD38, or CD154.

194. The method of any one of claims 1 to 193 wherein the one or more activation markers is selected from the group consisting of CD137, CD69, CD25, Ki67, and CD107, or a combination of thereof.

195. The method of any one of claims 1 to 194, wherein the algorithm is a cluster analysis algorithm.

196. The method of any one of claims 1 to 195, wherein the algorithm comprises t-distributed stochastic neighbor embedding.

197. The method of any one of claims 1 to 196, wherein the measured functional response to P comprises detection of one or more activation markers and/or one or more secreted molecules.

198. The method of any one of claims 1 to 197, wherein the one or more activation markers comprises CD137, CD69, CD25, Ki67, CD107, CD122, CD27, CD28, CD95, CD134, killer-cell lectin like receptor G1 (KLRG1), CD38, or CD154.

199. The method of any one of claims 1 to 198, wherein the one or more activation markers is selected from the group consisting of CD137, CD69, CD25, Ki67, and CD107, or a combination of thereof.

200. The method of any one of claims 1 to 199, wherein the one or more secreted molecules comprises one or more cytokines.

201. The method of any one of claims 1 to 200, wherein the one or more cytokines is interferon gamma (IFN-gamma), tumor necrosis factor alpha (TNFalpha), interleukin-2 (IL-2), or a combination of two or more of the foregoing.

202. The method of any one of claims 1 to 201, wherein the one or more secreted molecules comprises granzyme.

203. The method of any one of claims 1 to 202, wherein the one or more secreted molecules comprises perforin.

204. The method of any one of claims 1 to 203, wherein the measured functional response to P comprises detection of T cell proliferation.

205. The method of any one of claims 1 to 204, wherein the first plurality of T cells and the second plurality of T cells are derived from a common starting population of PBMCs.

206. The method of any one of claims 1 to 205, wherein the plurality of expression rate profiles are obtained from a series of single-cell transcriptome analyses.

207. The method of any one of claims 1 to 206, wherein T cells in the one of the plurality of T cell clusters express the predetermined first activation marker at an average second expression rate that exceeds a first expression rate threshold.

208. The method of any one of claims 1 to 207, wherein T cells in the one of the plurality of T cell clusters express the second activation marker at an average second expression rate that exceeds a second expression rate threshold.

209. The method of any one of claims 1 to 208, wherein the method further comprises identifying the plurality of P-binding T cells by matching T cell receptor sequences of the plurality of P-binding T cells to predetermined T cell receptor sequences.

210. The method of any one of claims 1 to 209, wherein the predetermined T cell receptor sequences are determined by sequencing a second plurality of T cells bound to P-loaded MHC proteins.

211. The method of any one of claims 1 to 210, wherein the activation marker is not expressed or is downregulated in at least two other T cells present in another one of the plurality of T cell clusters, wherein the at least two other T cells do not show a functional response when measured.

212. The method of any one of claims 1 to 211, wherein the candidate antigen is a neoantigen.

213. The method of any one of claims 1 to 212, wherein the antigen-specific vaccine is for treatment of a cancer.

214. The method of any one of claims 1 to 213, wherein the predetermined type of antigen is a neoantigen.

215. The method of any one of claims 1 to 214, wherein the neoantigen is a peptide.

216. The method of any one of claims 1 to 215, wherein the peptide consists of 8-15 amino acids.

217. The method of any one of claims 1 to 216, wherein the peptide consists of 12-40 amino acids.

218. The method of any one of claims 1 to 217, wherein the neoantigen is derived from a tumor.

219. The method of any one of claims 1 to 218, wherein the tumor is a solid tumor.

220. The method of any one of claims 1 to 219, wherein the neoantigen is presented on a tumor.

221. The method of any one of claims 1 to 220, wherein the neoantigen is a personalized neoantigen.

222. The method of any one of claims 1 to 221, wherein the neoantigen is a shared tumor neoantigen.

223. The method of any one of claims 1 to 222, wherein the shared tumor neoantigen is a tumor associated peptide neoantigen.

224. The method of any one of claims 1 to 223, wherein the neoantigen is characteristic of a particular type of tumor.

225. The method of any one of claims 1 to 224, wherein the neoantigen is a tumor associated peptide neoantigen.

226. The method of any one of claims 1 to 225, wherein the neoantigen is selected from one or more neoantigens identified by a model.

227. The method of any one of claims 1 to 226, wherein the model is calibrated by machine learning.

228. The method of any one of claims 1 to 227, wherein the one or more neoantigens are personalized neoantigens.

229. The method of any one of claims 1 to 228, wherein the one or more neoantigens are present in a list of shared neoantigens.

230. The method of any one of claims 1 to 229, wherein the neoantigen is selected from one or more neoantigens identified by an artificial intelligence model.

231. The method of any one of claims 1 to 230, wherein the artificial intelligence model comprises a neural network.

232. The method of any one of claims 1 to 231, wherein the neoantigen is selected from a set of presentation likelihoods.

233. The method of any one of claims 1 to 232, wherein the personalized neoantigen is selected based on one or more of the machine learning methods, software, and/or systems disclosed in the INCORPORATED REFERENCES.

234. The method of any one of claims 1 to 233, wherein the predetermined type of antigen is a viral antigen.

235. The method of any one of claims 1 to 234, which is exclusive of in vitro priming.

236. A composition obtained by any one of the methods of claims 1 to 235.

237. A composition comprising an artificial T cell receptor selective to a predetermined type of antigen, comprising:
i) at least a portion of a CDR3 region selected by—
a) analyzing a mixture of natural T cells to identify antigen-binding T cells and antigen-activated T cells for the predetermined type of antigen; and
b) identifying at least a portion of at least one T cell receptor sequence shared by at least one of the antigen-binding T cells and at least one of the antigen-activated T cells, the at least a portion of at least one T cell receptor sequence containing the at least a portion of the CDR3 region; and
ii) a T cell receptor fragment.

238. A T cell comprising an artificial T cell receptor or a fragment thereof obtained by any one of the methods of claims 1 to 235.

239. The T cell of claim 238, for use in the treatment of cancer.

240. A kit comprising the composition of claim 236 or 237.

241. A kit for use in any one of the methods of claims 1 to 235.
